(19) United States
(12) Patent Application Publication
(10) Pub. No.: US 2007/0092889 A1
(43) Pub. Date: Apr. 26, 2007

(54) Parkinson's disease-related disease compositions and methods

(75) Inventors: David R. Cox, Belmont, CA (US); Demetrius M. Maraganore, Rochester, MN (US); Dennis G. Ballinger, Menlo Park, CA (US); Krishna Pant, San Jose, CA (US); Timothy G. Lesnick, Rochester, MN (US)

Correspondence Address: PERLEGEN SCIENCES, INC.;LEGAL DEPARTMENT, 2021 STIERLIN COURT, MOUNTAIN VIEW, CA, 94043, US

(73) Assignee: Perlegen Sciences, Inc., Mountain View, CA (US)

(21) Appl. No.: 11/446,599

(22) Filed: Jun. 2, 2006

Related U.S. Application Data
(60) Provisional application No. 60/686,947, filed on Jun. 2, 2005.

(51) Int. Cl. C12Q 1/68; G01N 33/53; C07H 21/04

(52) U.S. Cl. 435/6; 435/7.1; 536/23.2

(57) ABSTRACT

Compositions and methods for use in the therapeutic and preventative treatment, study, diagnosis and prognosis of PD-related disease are disclosed. Also provided are kits and reagents for prognosis and diagnosis of PD-related disease and related conditions.

### CROSS-REFERENCES TO RELATED APPLICATIONS

The present application is a nonprovisional of and claims the benefit of U.S. provisional patent application Ser. No. 60/686,947, filed Jun. 2, 2005, the disclosure of which is specifically incorporated herein by reference in its entirety for all purposes.

### STATEMENT OF GOVERNMENT INTEREST

This invention was made with government support under NIH Grant Nos. ES10751, ES10751-S1, NS33978 and NS40256. As such, the United States government has certain rights in the invention.

### BACKGROUND

Parkinson's disease (PD), the second most common neurodegenerative disorder, is characterized by tremor of the hands, arms, legs, jaw and face (resting tremor); muscular rigidity; slowness of movement (bradykinesia); and impaired balance and coordination (postural instability) (Hoehn, et al. (1967) “Parkinsonism: onset, progression and mortality”, Neurology 17:427-442). Individuals with PD may also experience additional symptoms, such as dysautonomia, dystonic cramps and dementia. Pathological features of PD include a loss of dopaminergic neurons in the substantia nigra (SN) and the presence of intracellular inclusions in surviving neurons in various areas of the brain (Pollanen, et al. (1993) “Pathology and biology of the Lewy body”, J Neuropathol Exp Neurol 52183-91; Kuzuhara, et al. (1988) “Lewy bodies are ubiquitinated: a light and electron microscopic immunocytochemical study”, Acta Neuropathol 75:345-353; Nussbaum, et al. (1997) “Genetics of Parkinson's disease”, Hum. Molec. Genet. 6:1687-1691). As many as one million Americans suffer from Parkinson's disease, and the prevalence among persons 65-69 is approximately 0.5 to 1 percent, rising to 1 to 3 percent among persons 80 years of age or older.

There is no known cure for PD. Patients are treated with drugs and physical therapy to control the symptoms, but the disease is a progressive disorder and symptoms continue to worsen throughout life. There are four major categories of drugs used to treat PD: Levodopa, direct dopamine agonists, catechol-O-methyltransferase (COMT) inhibitors and anticholinergics. Other types of drugs include selegiline (an MAO-B inhibitor), amantadine (an antiviral agent), vitamin E and hormone replacement therapy. Although these treatments may provide some relieve from the symptoms of PD, these noncurative drug treatments are often are accompanied by side effects, such as low blood pressure, nausea, constipation, and various psychiatric or behavioral disorders (e.g., hallucinations, depression, and sleep disorders).

Although the molecular bases for most cases of PD remain unknown, both genetic and environmental factors may play significant roles. For example, monozygotic twins with an onset of disease before the age of 50 years have a high rate of concordance, and an increased risk of PD is also seen among the first-degree relatives of patients, indicating that there is a genetic component to the disease (Tanner, et al. (1999) “Parkinson disease in twins: an etiologic study”, JAMA 281:341-346; Duvoisin, et al., (1992) “Hereditary Lewy-body parkinsonism and evidence for a genetic etiology of Parkinson's disease”, Brain Pathol 2:309-320; Marder, et al. (1996) “Risk. of Parkinson's disease among first-degree relatives: a community-based study”, Neurology 47:155-160; and Payami, et al. (1994) “Increased risk of Parkinson's disease in parents and siblings of patients”, Ann Neurol 36:659-661). In contrast, some studies indicate that environmental factors may be more important than genetic factors in familial PD (Calne et al. (1987) “Familial Parkinson's disease: possible role of environmental factors”, Canad J Neurol Sci 14:303-305; Teravainen et al. (1986) “The age of onset of Parkinson's disease: etiological implications”, Canad J Neurol Sci 13:317-319; Calne et al. (1983) “Aetiology of Parkinson's disease”, Lancet II: 1457-1459; and Barbeau et al. (1985) “Ecogenetics of Parkinson's disease: 4-hydroxylation of debrisoquine”, Lancet II: 1213-1216). For example, parkinsonism was found to be linked to meperidine drug use (Langston et al. (1983)). For a review, see Warner, et al. (2003) “Genetic and Environmental Factors in the Cause of Parkinson's Disease”, Ann Neurol 53 (suppl 3):S16-S25.

Several genetic regions have been found to be associated with PD. The PARK1 region at 4q21 contains the α-synuclein (SNCA) gene. Certain mutations in this gene confer a very rare autosomal dominant form of PD (Duvoisin, R. C. (1996) “Recent advances in the genetics of Parkinson's disease”, Adv Neurol 69:33-40; Polymeropoulos et al. (1997) “Mutation in the alpha-synuclein gene identified in families with Parkinson's disease”, Science 276:2045-7; and Kruger et al. (1998) “Ala30Pro mutation in the gene encoding alpha-synuclein in Parkinson's disease”, Nat Genet 18:106-108). The PARK2 region at 6q25-27 contains the Parkin gene. The loss of function of both copies of the parkin gene confers an autosomal recessive juvenile form of PD (Abbas, et al. (1999) “A wide variety of mutation in the parkin gene are responsible for autosomal recessive parkinsonism in Europe”, Hum Mol Genet 8:567-574; Lucking, et al. (1998) “Homozygous deletions in the parkin gene in European and North African families with autosomal recessive juvenile parkinsonism”, Lancet 352:1355-1356; and Lucking et al. (2000) “Association between early-onset Parkinson's disease and mutations in the parkin gene”, N Engl J Med 342:1560-1567). Other regions believed to contain one or more genes associated with PD include PARK3 at 2p13 (autosomal dominant), PARK4 at 4p15 (autosomal dominant; same locus as PARK1), PARK5 at 4p14 (which contains a gene encoding a neuron-specific C-terminal ubiquitin hydrolase), PARK6 at 1p35 (autosomal recessive), PARK7 at 1p36 (which contains the DJ-1 gene; autosomal recessive) and PARK8 at 12p1.2-q13.1 (which contains the LRRK2 gene; autosomal dominant). Additional loci designated PARK9, PARK10 and PARK 11have also been linked to PD. While the molecular bases for most cases of PD are unclear, the various genetic regions that have been linked to this devastating disease serve to illustrate the potential that the etiology of PD may involve the interaction of a large number of genetic components.

### BRIEF SUMMARY

Methods and compositions for use in diagnostics, prognostics, therapeutics, prevention, treatment and study of neurodegenerative disorders, in particular Parkinson's disease and Alzheimer's disease, are provided.

### DETAILED DESCRIPTION

It is understood that the terminology used herein is for the purpose of describing particular embodiments only and is not intended to limit the scope of the present invention.

The term “a” or “an” as used herein may mean one or more.

The term “another” as used herein may mean at least a second or more.

The term “associated gene” or “PD-related disease gene” refers to a gene, a genomic region 10 kb upstream and 10 kb downstream of such gene, and regulatory regions that modulate the expression of such gene, comprising at least a portion of one of the polymorphic regions identified in Tables 1 and 2, and all associated gene products (e.g., isoforms, splicing variants, and/or modifications, derivatives, etc.). The sequence of a PD-related disease gene may contain one or more PD-related disease polymorphisms. For example, the sequence of a. PD-related disease gene in an individual may contain one or more reference or alternate alleles, or may contain a combination of reference and alternate alleles, or may contain alleles in linkage disequilibrium with one or more of the polymorphic regions identified in Tables 1 and 2.

The term “associated gene pathway” generally refers to genes and gene products comprising a PD-related disease pathway, and may include one or more genes that act upstream or downstream of an associated gene in a PD-related disease pathway; or any gene whose gene product interacts with, binds to, competes with, induces, enhances or inhibits, directly or indirectly, the expression or activity of an associated gene; or any gene whose expression or activity is induced, enhanced or inhibited, directly or indirectly, by an associated gene; or any gene whose gene product is induced, enhanced or inhibited, directly or indirectly, by an associated gene. An associated gene pathway may refer to one or more genes.

The term “derivative” refers to chemical modification of a nucleic acid, a protein or mimetic thereof. Examples of chemical modifications of a nucleic acid include replacement of hydrogen by an alkyl, an acyl or an amino group. A nucleic acid derivative may also refer to a nucleic acid that was derived from another nucleic acid (e.g., mRNA transcribed from a gene, cDNA synthesized from an RNA molecule, or cRNA synthesized from a DNA molecule, etc.). A nucleic acid derivative can encode a polypeptide that retains, changes, inhibits or enhances essential characteristics or functions of the polypeptide that the natural nucleic acid encodes. A polypeptide derivative is one that is modified by glycosylation, pegylation or other process, and that retains, changes, inhibits or enhances at least one characteristic or function (e.g., immunological response) of the polypeptide from which it was derived.

The term “stringent conditions” refers to conditions for hybridization of complementary nucleic acids wherein the presence of such hybridization may be detected. For example, the detection of hybridization may be used as a proxy for determining the presence of a particular nucleic acid. Different stringency conditions may be utilized under different circumstances. Stringent conditions depend on, for example, length of the nucleic acids, hybridization temperature, buffers, and other hybridization reaction conditions. Generally, stringent conditions are selected to be about 5° C. lower than the thermal melting point (Tm) of a specific sequence at a defined ionic strength and pH. The Tm is the temperature (under defined ionic strength, pH and nucleic acid concentration) at which 50% of the complementary nucleic acids hybridize to a target nucleic acid at equilibrium. As target nucleic acids are generally. present in excess, at Tm, 50% of the complementary nucleic acids are occupied at equilibrium. Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide. For example, in some aspects, conditions of 5×SSPE (750 mM NaCl, 50 mM NaPhosphate, 5 mM EDTA, pH 7.4) and a temperature of 25-30° C. are suitable for allele-specific nucleic acid hybridizations. In other aspects, conditions of 1M TMACL (tetramethylammonium chloride), 3.25 M Tris (pH 7.8 or 8), 0.00325% Triton X-100, and a temperature of 50° C. are suitable for allele-specific nucleic acid hybridizations.

The terms “isolated” and “purified” refer to a material that is substantially or essentially removed from or concentrated in its natural environment. For example, an isolated nucleic acid is one that is separated from the nucleic acids that normally flank it or from other biological materials (e.g., other nucleic acids, proteins, lipids, cellular components, etc.) in a sample. In another example, a polypeptide is purified if it is substantially removed from or concentrated in its natural environment.

The term “PD-related disease” refers to one or more diseases, conditions or symptoms or susceptibility to diseases, conditions or symptoms that involve directly or indirectly, neurodegeneration including but not limited to the following: Alzheimer's disease, amyotrophic lateral sclerosis (ALS), Alpers' disease, Batten disease, Cockayne syndrome, corticobasal ganglionic degeneration, Huntington's disease, Lewy body disease, Pick's disease, motor neuron disease, multiple system atrophy, olivopontocerebellar atrophy, Parkinson's disease, postpoliomyelitis syndrome, prion diseases, progressive supranuclear palsy, Rett syndrome, Shy-Drager syndrome and tuberous sclerosis. In certain aspects, a PD-related disease is a neurodegenerative disease the affects neurons in the brain. A PD-related disease may be e.g. a condition that is a risk factor for developing PD, or may be a condition for which PD is a risk factor, or both.

The term “PD-related disease nucleic acid” means a nucleic acid, or fragment, derivative (e.g., RNA), variant, polymorphism, or complement thereof, associated with resistance or susceptibility to PD-related disease including, for example, at least one or more PD-related disease polymorphisms, genomic regions spanning 10 kb immediately upstream and 10 kb immediately downstream of a PD-related disease polymorphism, coding and non-coding regions of an associated gene, and/or genomic regions spanning 10 kb immediately upstream and 10 kb immediately downstream of an associated gene, and nucleotide variants thereof. The term also includes nucleic acids similarly related to genes in an associated gene pathway. A PD-related disease nucleic acid may also be an “associated genomic region” when it is found within the genome of an organism.

The term “PD-related disease polymorphism” or “associated polymorphism” refers to a specific nucleic acid locus at which a nucleotide polymorphism associated with PD-related disease occurs. For example, a PD-related disease polymorphism may be a SNP position such as those provided in Tables 1 and 2. There may be two or more nucleotide base variants (“alleles”) at a given PD-related disease polymorphism, and each of these alleles may be specifically associated with either a resistance or a susceptibility to PD-related disease, or to a response to a treatment regimen (e.g., drug response). An allele that is the same as that found in a reference nucleic acid sequence is referred to as a “reference allele”, and an allele that is different than that found in the reference sequence is referred to as an “alternate allele”.

The term “PD-related disease polypeptide” or “associated polypeptide” refers to any peptide, polypeptide, or fragment, derivative or variant thereof, associated with susceptibility or resistance to PD-related disease, including a peptide or polypeptide regulated or encoded, in whole or in part, by an associated gene or genomic regions of 10 kb immediately upstream and downstream of an associated gene, or fragment, variants, derivative, or modifications thereof. The term also includes such polypeptides up- or down-stream in an associated gene pathway.

The term “PD-related disease haplotype block” refers to a haplotype block comprising at least one PD-related disease polymorphism. A PD-related disease haplotype block comprises at least two or more haplotype patterns, each of which may be specifically associated with either a resistance or a susceptibility to PD-related disease, or to a response to a treatment regimen (e.g., drug response).

The term “modulate” refers to a change such as in expression, lifespan, or function such as an increase, decrease, alteration, enhancement or inhibition of expression or activity.

The term “nucleic acid,” refers to a deoxyribonucleotide or ribonucleotide, whether singular or in polymers, naturally occurring or non-naturally occurring, double-stranded or single-stranded, translated (e.g., gene) or untranslated (e.g. regulatory region), or any fragments, derivatives, mimetics or complements thereof. A nucleic acid includes analogs (e.g., phosphorothioates, phosphoramidates, methyl phosphonate, chiral-methyl phosphonates, 2 -O-methyl ribonucleotides) or modified nucleic acids (e.g., modified backbone residues or linkages) or nucleic acids that are combined with carbohydrates, lipids, protein or other materials, or peptide nucleic acids (PNAs) (e.g., chromatin, ribosomes, transcriptosomes, etc.) or nucleic acids in various structures (e.g., A DNA, B DNA, Z-form DNA, siRNA, tRNA, ribozymes, etc.) A nucleic acid can include one or more polymorphisms, variations or mutations (e.g., SNPs, insertions, deletions, inversions, translocations, etc.) Examples of nucleic acids include oligonucleotides, nucleotides, polynucleotides, nucleic acid sequences, genomic sequences, antisense nucleic acids, DNA regions, probes, primers, genes, regulatory regions, introns, exons, open-reading frames, binding sites, target nucleic acids and allele-specific nucleic acids.

The term “polymorphism” refers a position in a nucleic acid or polypeptide that possesses the quality or character of occurring in several different forms. A nucleic acid or polypeptide may be naturally or non-naturally polymorphic, e.g., having one or more sequence differences (e.g., additions, deletions and/or substitutions) as compared to a reference sequence. A reference sequence may be based on publicly available information (e.g., the U.C. Santa Cruz Human Genome Browser Gateway (genome.ucsc.edu/cgi-bin/hgGateway) or the NCBI website (www.ncbi.nlm.nih.gov)) or may be determined by a practitioner of the present invention using methods well known in the art (e.g., by sequencing a reference nucleic acid). A nucleic acid polymorphism is characterized by two or more “alleles”, or versions of the nucleic acid sequence. Typically, an allele of a polymorphism that is identical to a reference sequence is referred to as a “reference allele” and an allele of a polymorphism that is different from a reference sequence is referred to as an “alternate allele”, or sometimes a “variant allele”. However, as any two reference sequences may differ at a polymorphic locus, an “alternate allele” may be found in a reference sequence and a “reference allele” may not. Furthermore, the designation of a “reference allele” and an “alternate allele” need not be based on any particular reference sequence and can be performed arbitrarily simply as a means to distinguish between two different alleles of a polymorphism. As such, the designation of alleles provided herein as “reference” or “alternate” should not be construed to indicate that the allele is or is not present in a particular reference sequence. A nucleic acid comprising an alternate allele may be referred to as a “variant nucleic acid”. Nucleic acid polymorphisms include loci within nucleic acids encoding a polypeptide, but which due to the degeneracy of the genetic code are not found in nature. A polypeptide polymorphism is characterized by two or more versions of an amino acid sequence, with a version that is identical to a reference sequence referred to as a “reference polypeptide” and a version that is different from a reference sequence referred to as an “alternate polypeptide” or a “polypeptide variant”. Polypeptide polymorphisms include polypeptides encoded by another locus in the human genome or other organism's genome that have substantial homology, in whole or in part, to the polypeptides provided herein. The term “synonymous polymorphism” refers to a polymorphism in a coding region of a gene for which different alleles of the polymorphism encode an identical amino acid sequence. The term “non-synonymous polymorphism” refers to a polymorphism in a coding region of a gene for which different alleles of the polymorphism encode different amino acid sequences. Non-synonymous polymorphisms may be conservative or non-conservative. A “conservative polymorphism” refers to a non-synonymous polymorphism for which the different amino acid sequences encoded are is functionally equivalent. A “non-conservative polymorphism” refers to a non-synonymous polymorphism for which the different amino acid sequences encoded are functionally dissimilar. “Functionally equivalent” as used herein refers to a polypeptide capable of exhibiting a substantially similar activity as another polypeptide.

The terms “polypeptide,” “peptide,” “oligopeptide” and “protein” are used interchangeably to refer to a polymer of amino acids, PNAs or mimetics, of no specific length and to all fragments, isoforms, variants, derivatives and modifications thereof. A polypeptide may be naturally or non-naturally occurring. The term variant when used to describe a polypeptide refers to variations in amino acid sequences as compared to a reference polypeptide sequence, whether or not such variations are encoded by conservative or non-conservative polymorphisms, for example. An amino acid substitution that is encoded by a conservative polymorphism may be referred to as a conservative substitution. Likewise, an amino acid substitution that is encoded by a non-conservative polymorphism may be referred to as a non-conservative substitution. The term modification includes tags, labels, post-translational modifications or other chemical or biological modifications. In preferred embodiment a polypeptide is purified.

The term “probes” or “primers” refers to nucleic acids that can hybridize, in whole or in part, in a base-specific manner to a complementary strand. Typically, the term “primer” refers to a single-stranded nucleic acid that acts as a point of initiation of template-directed DNA synthesis (e.g., PCR primers) and the term “probe”refers to a single-stranded nucleic acid designed to hybridize to a target nucleic acid. For example, hybridization of the probe to a target nucleic acid may be used to purify the target nucleic acid, or detection of hybridization (or lack thereof) of the probe to a target nucleic acid may be used to determine the presence (or absence) of the target nucleic acid in a sample. Although single-stranded probes and primers are primarily discussed herein, the present invention is not limited to such probes and primers; double-stranded or partially double-stranded probes or primers are also included.

The term “specific hybridization” refers to the ability of a first nucleic acid to bind, duplex or hybridize to a second nucleic acid in a manner such that the second nucleic acid can be identified or distinguished from other components of a mixture (e.g., cellular extracts, genomic DNA, etc.) In certain embodiments, specific hybridization is performed under stringent-conditions.

The term “substrate” refers to any rigid or semi-rigid support to which molecules (e.g., nucleic acids, polypeptides, mimetics) may be bound. Examples of substrates include membranes, filters, chips, slides, wafers, fibers (e.g., optical fibers), magnetic or nonmagnetic beads, gels, capillaries, or other tubing, plates, polymers, and microparticles with a variety of surface forms including wells, trenches, pins, channels and pores, and may be manufactured from various substances, including but not limited to glass, silicon, fused silica, borosilicate, quartz, soda lime glass, a polymeric material (e.g., polyethylene, polycarbonate, polyvinylchloride, polystyrene, and the like) or a combination thereof.

The term “vector” refers to any construct or composition by which the expression, transfer or manipulation of a nucleic acid may be accomplished or facilitated. For example, the term vector can be an artificial chromosome (e.g., BAC, YAC, etc.), cosmid, viral particle, viral nucleic acid, plasmid, or a liposome. For example, in some embodiments a vector is a viral nucleic acid or a plasmid with appropriate transcription/translation control signals. An expression vector is a vector that is designed to promote the expression of one or more nucleic acid inserts.

I. PD-Related Disease Nucleic Acids

Results from various studies have revealed monozygotic twins with an onset of disease before the age of 50 years have a high rate of concordance, and an increased risk of PD is also seen among the first-degree relatives of patients. These findings suggest that there are specific genetic factors that influence an individual's susceptibility and resistance to PD and PD-related diseases. Association-based genome scans provide localizing information that is much more precise (often extending over a few thousand base pairs) than the corresponding information from linkage-based studies (which often extend over many million base pairs).

An association study revealed, by comparing case and control groups, that the polymorphisms identified in Tables 1 and 2 in Appendix 1, and surrounding genomic regions (or regions in linkage disequilibrium with the polymorphisms of Tables 1 and 2) are associated with susceptibility and/or resistance to PD-related disease (i.e., are PD-related disease polymorphisms). It is believed that individuals who are resistant or susceptible to PD-related disease exhibit a different-expression pattern of PD-related disease polypeptides and/or PD-related disease nucleic acids. This different expression pattern may be a difference in the particular alleles or variants expressed by these individuals, or may be a difference in the absolute level of expression of the PD-related disease polypeptides or nucleic acids.

Tables 1 and 2 in Appendix 1 identify PD-related disease polymorphisms. In particular, Table 1, column 1 identifies a SNP identification number for each polymorphism. Table 1, column 2 identifies the accession number of the contig to which the SNP aligns from NCBI Build 34 of the human genome. Table 1, column 3 identifies the position for each polymorphism within the NCBI Build 34 contig. For SNPs that lie within or near a gene, Table 1, column 4 identifies the Gene database ID (NCBI); Table 1, column 5 provides the Gene database name (NCBI); and Table 1, column 6 provides the location of the SNP relative to the gene. Specifically, “up” means that the SNP occurs within 10 kb upstream of the gene, unless otherwise specified; “down” means that the SNP occurs within 10 kb downstream of the gene; “outsideCodingRegion-5′UTR” means that the SNP occurs in the 5′ untranslated region of the gene; “outsideCodingRegion-3′UTR” means that the SNP occurs in the 3′ untranslated region of the gene; “intron” means that the SNP occurs within an intron of the gene; “nonsynonymous coding change” means that the SNP occurs within the coding region of the gene, and that the reference allele of the SNP encodes a polypeptide with an amino acid that is different than that encoded by the alternate allele of the SNP; and “synonymous coding change” means that the SNP occurs within the coding region of the gene, and that the reference allele of the SNP encodes a polypeptide with the same amino acid sequence as that encoded by the alternate allele of the SNP. In addition, Table 1, column 7 provides a description of the gene from the NCBI Gene database.

Table 1, column 8 identifies the difference in allele frequencies between the case population and the control population in all the original samples. Allele frequencies for each population may be calculated according to the methods in U.S. patent application Ser. No. 10/970,761, filed Oct. 20, 2004, entitled “Improved Analysis Methods and Apparatus for Individual Genotyping”. Other methods for calculating allele frequencies are detailed in, e.g., U.S. patent application Ser. No. 10/351,973, filed Jan. 27, 2003, entitled “Apparatus and Methods for Determining Individual Genotypes”; U.S. patent application Ser. No. 10/786,475, filed Feb. 24, 2004, entitled “Improvements to Analysis Methods for Individual Genotyping”; and 60/460,329, filed on Apr. 3, 2003, and U.S. patent application Ser. No. 10/768,788, filed Jan. 30, 2004, both of which are entitled “Apparatus and Methods for Analyzing and Characterizing Nucleic Acid Sequences”, all of which are incorporated herein by reference in their entireties for all purposes.

The “difference in allele frequency” (also termed “delta P” or “ΔP”) is the frequency of the reference allele in the cases minus the frequency of the reference allele in the controls. A positive difference in allele frequency indicates that the reference allele is associated with the case group (e.g., the reference allele is associated with susceptibility to PD-related disease), and a negative difference in allele frequency indicates that the alternate allele is associated with the case group (e.g., the alternate allele is associated with susceptibility to PD-related disease). Thus, based on the sign of the difference in allele frequency, one can identify which allele is associated with susceptibility to PD-related disease and which allele is not, wherein the allele that is not associated with susceptibility may be associated with protection from PD-related disease. (The reference and alternate alleles are provided in Table 2, as described below.)

Table 1, column 9 provides a p-value from the sibling TDT test in the original samples. Table 1, column 10 provides the difference in allele frequency between the case population and the control population in all the replication samples. Table 1, column 11 provides the p-value for all the replication samples. Table 1, column 12 provides the combined p-value for the original and replication samples.

Table 2 in Appendix 1 provides some of the same information as does Table 1. For example, in Table 2, column 1 identifies a SNP identification number for each polymorphism; column 8 identifies the accession number of the contig from NCBI Build 34 of the human genome to which the SNP aligns; and column 9 identifies the position for each polymorphism within the NCBI Build 34 contig. In addition, Table 2, column 2 provides the RefSnp rsID from NCBI, and column 3 provides the RefSnp ssID for the SNPs that Perlegen submitted to dbSNP, when these IDs are available. Table 2, columns 4 and 5 identify the reference and alternate alleles for the SNP, respectively, the reference allele being defined as the allele on NCBI Build 34. Additional polymorphisms can be used in addition to those identified in Table 1, which can be identified according to U.S. patent application Ser. No. 10/106,097, filed Mar. 26, 2002, entitled “Methods for Genomic Analysis”; U.S. patent application Ser. No. 10/284,444, filed Oct. 31, 2002, entitled “Human Genomic Polymorphisms”; and Patil, et al. (2001) “Blocks of Limited Haplotype Diversity Revealed by High-Resolution Scanning of Human Chromosome 21”, Science 294:1719-1723. Polymorphisms in a haplotype block with a PD-related disease polymorphism are also PD-related disease polymorphisms. Further, if a haplotype pattern contains an allele of a PD-related disease polymorphism, and the allele is associated with resistance to PD-related disease, then all other alleles in that haplotype pattern are also associated with resistance to PD-related disease; similarly, if a haplotype pattern contains an allele of a PD-related disease polymorphism, and the allele is associated with susceptibility to PD-related disease, then all other alleles in that haplotype pattern are also associated with susceptibility to PD-related disease.

Table 2, column 6 identifies the chromosome for the NCBI Build 34 contig on which the best alignment was found,.and Table 2, column 7 highlights whether the contig is autosomal or sex-linked. Table 2, column 10 identifies whether the reference allele corresponds to the “+” or “−” strand of NCBI Build 34. Table 2, column 11 identifies the 14 nucleotide bases upstream and 14 nucleotide bases downstream of each PD-related disease polymorphism, with the center position (designated “N”) containing the reference allele. This 29mer is the sequence that was used to assay the SNP on a microarray.

SNP 829480 is located on chromosome 5 at position 9385019 in an intron of the SEMA5A (semaphoring A5A) gene (sema domain, seven thrombospondin repeats (type 1 and type 1-like), transmembrane domain (™) and short cytoplasmic domain, (semaphorin)5A), which has not been previously implicated in PD-related disease. This gene maps to the deletion candidate interval for the cri-du-chat syndrome. This chromosomal deletion syndrome is associated with severe abnormalities in brain development with resultant mental retardation (Simmons et al., Molecular cloning and mapping of human semaphorin F from the Cri-du-chat candidate interval. Biochem Biophys Res Commun. 1998 Jan 26;242(3):685-91). The encoded semaphorin protein has receptor activity and contributes to neurogenesis and apoptotic processes (e.g., neuronal apoptosis). It elicits multiple functional responses through its receptor plexin-B3 (Artigiani et al., Plexin-B3 is a functional receptor for semaphorin 5A. Embo reports 2004;5:710-714).

Semaphorins, in general, are transmembrane proteins that have been implicated in many processes of neuronal-development, such as development of the mesencephalic dopamine neuron system, axonal fasciculation, target selection, neuronal migration, dendritic guidance, axon guidance, as well as in remodeling and repair of the adult nervous system, including axon guidance during neurogenesis and inhibition of axon regeneration (Kawano et al. (2003) “Aberrant trajectory of ascending dopaminergic pathway in mice lacking Nkx2.1”, Exp Neurol 182:103-112; He, et al. (2002) “Knowing How to Navigate: Mechanisms of Semaphorin Signaling in the Nervous System”, Science's STKE: www.stke.org/cgi/content/full/OC_sigtrans; 2002/119/rel). Semaphorins have also been implicated as mediators of neuronal apoptosis, and antibodies directed against semaphorins inhibit dopamine-induced neuronal death (Shirvan, et al. (1999) “Semaphorins as Mediators of Neuronal Apoptosis”, J Neurochem 73(3):961-971; Barzilai, et al. (2000) “The molecular mechanism of dopamine-induced apoptosis: identification and characterization of genes that mediate dopamine toxicity”, J Neural Transm 60:59-76; and Shirvan, et al. (2000) “Induction of neuronal apoptosis by Semaphorin3A-derived peptide”, Mol Brain Res 83:81-93), a hallmark of PD-related disease. The expression of semaphorin genes is abnormal in Alzheimer's disease, which shares clinical, pathological, and etiological features with PD (Hirsch et al. (1999) “Distribution of semaphorin IV in adult human brain”, Brain Res 823:67-79). It has also been suggested in a rat model of Parkinson's disease that the neuroprotective effects of vascular endothelial growth factor may be mediated via semaphorin inhibition (Yasuhara et al. (2004) “Neuroprotective effects of vascular endothelial growth factor (VEGF) upon dopaminergic neurons in a rat model of Parkinson's disease”, Eur J Neurosci 19:1494-1504). To date, there have been no clinical studies of semaphorin inhibitors as neuroprotective therapy for Parkinson's disease. The findings presented herein support the research and development of therapies targeting semaphorins as neuroprotection for PD-related disease.

Further, semaphorins may function not only as signaling ligands, but also as signal transducing receptors (He, et al., supra). In particular, G proteins are linked to semaphorin signaling via the GIPC (Ga-interacting protein (GAIP) interacting protein, COOH terminus) protein, a cytoplasmic protein found at clathrin-coated vesicles and involved in G protein-coupled signaling and vesicle trafficking. As a PDZ protein, GIPC acts to determine the subcellular localization of its interacting proteins; specifically, PDZ proteins interact with the cytoplasmic tails of transmembrane proteins to localize such proteins in the membrane. In particular, GIPC has been found to regulate the distribution of SEMA5A protein, causing it to aggregate in the plasma membrane; thus, it is believed that GIPC links SEMA5A to G protein signal transduction pathways (Wang, et al. (1999) “A PDZ Protein Regulates the Distribution of the Transmembrane Semaphorin, M-SemF”, J Biol Chem 274(20):14137-14146). Dopamine receptors are G protein coupled receptors that mediate dopamine activities in the brain and perpheral tissues. GIPC has been found to interact with two different dopamine receptors, D2R and D3R, both of which are expressed in the neurons of the striatum, a site of dopaminergic degeneration in the Parkinson's disease (Jeanneteau, et al. (2004) “Interactions of GIPC with Dopamine D₂, D₃ but not D₄ Receptors Define a Novel Mode of Regulation of G Protein-coupled Receptors”, Mol Biol Cell 15:696-705). Further, GIPC has also been found to recruit GAIP to attenuate D₂R signaling (Jeanneteau, et al. (2004) “GIPC Recruits GAIP (RGS 19) To Attenuate Dopamine D₂ Receptor Signaling”, Mol Biol Cell 15:4926-4937). Codistribution of GAIP, GIPC and D₂R in areas of the brain highly populated with dopaminoceptive and dopaminergic neurons (e.g., the striatum, substantia nigra, and ventral tegmental area). The roles of SEMA5A in neural development and/or apoptosis, as a signaling ligand and/or a signal transducing receptor, or its interaction with GIPC and/or its intracellular distribution may play a role in the development of PD-related disease in an individual.

SNP 1032590 and SNP 1032596 are located on chromosome 1 at positions 54007335 and 54015180, respectively. Both of these SNPs are in genomic regions upstream (28.4 kb and 20.6 kb, respectively) of the MRPL37 (mitochondrial ribosomal protein L37) gene. Encoded by nuclear genes, mammalian mitochondrial ribosomal proteins facilitate protein synthesis within the mitochondrion. Mitochondrial ribosomes (mitoribosomes) consist of a small 28S subunit and a large 39S subunit. MRPL37 encodes a 39S subunit protein of a mitochondrial ribosome. In addition, these SNPs are also near or within a second gene, LOC200008 (previously LOC401952); SNP 1032590 is upstream of the gene, and SNP 1032596 is in an intron of the gene. The function of this gene is unknown. The involvement of SNP 1032590 and/or SNP 1032596 in the regulation or expression of either of these genes may underlie their association with PD-related disease.

SNP 3413996 and SNP 3413959 are located on chromosome 4 at positions 103308314 and 103372790, respectively (both intronic positions) in the BANKI (B-cell scaffold protein-with ankyrin repeats) gene. BANK, a substrate of protein tyrosine kinases (PTKs), is expressed in B cells and is tyrosine phosphorylated upon B-cell antigen receptor (BCR) stimulation (Yokoyama, et al. (2002) “BANK regulates BCR-induced calcium mobilization by promoting tyrosine phosphorylation of IP3 receptor”, EMBO 21:83-92). BANK was implicated as a scaffold protein regulating BCR-induced calcium mobilization by connecting PTKs to IP₃R (inositol 1,4,5-triphosphate receptor).

SNP 1225958 is located on chromosome 4 at position 11699188 in an intron of at least one alternatively spliced transcript variant of the GREB1 (gene regulated by estrogen in breast cancer) gene, and in a genomic region upstream of at least one other alternatively spliced transcript variant of the GREB1 gene. GREB1 is an estrogen-responsive gene. that is an early response gene in the estrogen receptor-regulated pathway and is thought to play an important role in hormone responsive tissues and cancer. No less than three alternatively spliced transcript variants encoding distinct isoforms have been found for this gene.

SNP 1590687 is located on chromosome 7 at position 142679898 in the coding region of the OR10AC1P (LOC392133) gene, which is believed to be a G-protein-coupled olfactory receptor involved in initiation of neuronal responses in response to interaction with odorant molecules.

SNP 651340 is located on chromosome 1 at position 13502524 in an intron of the PRDM2 (PR domain containing 2, with ZNF domain) gene, which is a tumor suppressor gene that encodes a zinc finger that can bind to retinoblastoma protein, estrogen receptor and the TPA-responsive element (MTE) heme-oxygenase-1 gene. One of its putative functions is a role in transcriptional regulation during neuronal differentiation, and the gene is also cancer-related and estrogen-related. The involvement of this gene in PD-related disease may be related to the fact that endogenous estrogen and estrogen treatment may be protective against Parkinson's disease (Benedetti et al. Hysterectomy, menopause, and estrogen use preceding Parkinson's disease. Mov Disord 2001;16:830-837).

SNP 1427591 and SNP 1427562 are located on chromosome 2 at positions 225609523 and 225588397, respectively (both intronic positions) in the CUL3 gene, which is involved in targeting proteins for degradation via ubiquitination.

SNP 754013 is located on chromosome 2 at position 32459104 in an intron of the CARD12 (caspase recruitment domain family, member 12) gene, which is a Ced4 family member that can induce apoptosis, a process underlying many neurodegenerative disorders. In addition, CARD12 coprecipitates caspase-1, a caspase that participates in both apoptotic signaling and cytokine processing (Geddes, et al. (2001) “Human CARD12 is a novel CED4/Apaf-1 family member that induces apoptosis”, Biochem Biophys Res Commun 284(1):77-82).

SNP 879618 is located on chromosome 9 at position 130753983 in an intron of the DDX31 (DEAD (Asp-Glu-Ala-Asp) box polypeptide 31) gene, which is a DEAD box protein whose function has not yet been determined. In general, DEAD box proteins are putative RNA helicases implicated in a number of cellular processes involving alteration of RNA secondary structure such as translation initiation, nuclear and mitochondrial splicing, and ribosome and splicesome assembly. Some members of the DEAD box protein family are believed to be involved in embryogenesis, spermatogenesis and cellular growth and division.

SNP 1146860 is located on chromosome 1 at position 176304501 in the 3′ UTR noncoding region of the TOR3A (torsin family 3, member A) gene.

SNP 821239 is located on chromosome 17 at position 49155418 in an intron of the CACNA1G (calcium channel, voltage-dependent, T-type, alpha-1G subunit) gene, which is a T-type low voltage activated calcium channel (U.S. Pat. No. 6,358,706) T-type calcium channels are thought to be involved in neuronal oscillations and resonance, as well as pacemaker activity, low-threshold calcium spikes, and rebound burst firing. CACNA1G is predominantly expressed in the brain, especially in thalamus, cerebellum, substantia nigra, and frontal and occipital lobes (Monteil, et al. (2000) “Molecular and Functional Properties of the Human α_{1G} Subunit That Forms T-type Calcium Channels”, J Biol Chem 275(9):6090-6100). Enhanced expression of t-type calcium channels has been implicated in a variety of diseases, including hypertension and epilepsy. In addition, T-type calcium channel agonists have been described for use in treatment, control, amelioration or reduction of risk of a disease where abnormal oscillatory activity occurs in the brain, including depression, migraine, neuropathic pain, Parkinson's disease, phychosis and schizophrenia (WO 04/035000 A2). In fact, zonisamide, a drug shown to block T-type calcium channels in cultured neurons (Suzuki, et al. (1992) “Zonisamide blocks T-type calcium channel in cultured neurons of rat cerebral cortex”, Epilepsy Res 1:21-27), has been identified as a drug useful for treating or preventing neurodegenerative disorders such as Parkinson's disease, Huntington's disease, choreic syndrome and dystonic syndrome (U.S. Pat. No. 6,342,515; Murata, et al. (2001) “Zonisamide has beneficial effects on Parkinson's disease patients”, Neurosci Res 41(4):397-399; Okada, et al. (1995) “Effects of zonisamide on dopaminergic system”, Epilepsy Res 22(3):193-205; Murata, M. (2004) “Novel therapeutic effects of the anti-convulsant, zonisamide, on Parkinson's disease”, Curr Pharm Des 10(6):687-693; and Sobieszek,et al. (2003) “Zonisamide: A New Antiepileptic Drug”, Pol J Pharmacol 55:683-689).

SNP 2475361 is located on chromosome 9 at position 129031799 in an intron of the ABL1 (v-abl Abelson murine leukemia viral oncogene homolog 1) gene, which encodes a cytoplasmic and nuclear protein tyrosine kinase that has been implicated in processes of cell differentiation, cell division, cell adhesion and stress response. The activity of ABL1 is negatively regulated by its SH3 domain, and deletion of the SH3 domain turns ABL1 into an oncogene. Alterations of ABL1 by chromosomal rearrangement or viral transduction lead to malignant transformation; the t(9;22) translocation occurs in greater than 90% of chronic myelogeneous leukemia, 25-30% of adult and 2-10% of childhood acute lymphoblastic leukemia, and rare cases of acute myelogenous leukemia. The translocation results in the head-to-tail fusion of the BCR and ABL genes (Chissoe et al. (1995) “Sequence and analysis of the human ABL gene, the BCR gene, and regions involved in the Philadelphia chromosomal translocation”, Genomics 27:67-82). Further, ABL1 is involved in DNA damage-induced apoptosis (Truong, et al. (2003) “Modulation of DNA damage-induced apoptosis by cell adhesion is independently mediated by p53 and c-Ab1”,PNAS 100(18): 10281-10286).

SNP 1745110 is located on chromosome 11 at position 104415434 in an intron of the CASP5 (caspase 5, apoptosis-related cysteine protease) gene, which encodes a member of the cysteine-aspartic acid protease (caspase) family. Sequential activation of caspases plays a central role in the execution-phase of cell apoptosis. Overexpression of the active form of this enzyme has been shown to induce apoptosis in fibroblasts.

SNP 4040922 is located on chromosome 4 at position 154283058 in an intron of the ARFIP1 (ADP-ribosylation factor (ARF) interacting protein 1 (arfaptin 1)) gene. ARFs are implicated in vesicle budding and transport between endoplasmic reticulum and the Golgi complex. Specifically, ARFs regulate coatomer assembly on the Golgi as well as recruitment of clathrin adapter proteins ARFIP1 is an ARF-binding protein that inhibits vesicular trafficking and secretion.

SNP 2440329 is located on chromosome 9 at position 37958915 in an intron of the SHB (Src homology 2 domain containing adaptor protein B) gene, which encodes a protein involved in the regulation of cellular proliferation and apoptosis. SHB contains an SH2 domain, which interacts with tyrosine phosphorylation sites, implicating SHB in involvement in the signal-transduction of ligand-activated tyrosine kinase receptors (Welsh, et al. (1994) “Shb is a ubiquitously expressed Src homology 2 protein”, Oncogene 9:19-27). Adaptor proteins are molecules with multiple protein interaction motifs that do not appear to have catalytic activity on their own, but mediate the interaction of other proteins. The SHB gene encodes two such adaptor proteins from two different start methionines.

SNP 1549117 is located on chromosome 7 at position 6259487 in an intron of the KDELR2 (KDEL (Lys-Asp-Glu-Leu) endoplasmic reticulum protein retention receptor 2) gene is a homolog of a yeast protein, ERD2, a transmembrane protein involved in the retrieval of escaped endoplasmic reticulum (ER) resident proteins from the Golgi. Thus, KDELR2 is believed to provide signals that regulate retrograde traffic between the Golgi and the ER (Hsu, et al. (1992) “A brefeldin A-like phenotype is induced by the overexpression of a human ERD-2-like protein, ELP-1”, Cell 69(4):625-635.)

SNP 4355868 is located on chromosome 6 at position 162327821 in an intron of the PARK2 (Parkinson's disease (autosomal recessive, juvenile) 2, parkin) locus encodes the parkin protein. The loss of function of both copies of the parkin gene confers an autosomal recessive juvenile form of PD (Abbas, et al. (1999) “A wide variety of mutation in the parkin gene are responsible for autosomal recessive parkinsonism in Europe”, Hum Mol Genet 8:567-574; Lucking, et al. (1998) “Homozygous deletions in the parkin gene in European and North African families with autosomal recessive juvenile parkinsonism”, Lancet 352:1355-1356; and Lucking et al. (2000) “Association between early-onset Parkinson's disease and mutations in the parkin gene”, N Engl J Med 342:1560-1567). Parkin is involved in protein degradation as a ubiquitin-protein ligase, and this function may play a role in its involvement in the development of PD-related disease.

SNP 3336262 is located on chromosome 5 at position 158729574 in an intron of the IL12B (interleukin 12B (natural killer cell stimulatory factor 2, cytotoxic lymphocyte maturation factor 2, p40) gene, which encodesa subunit-of interleukin 12, a cytokine that acts on T and natural killer cells and has a broad array of biological activities. Overexpression of this gene was observed in the central nervous system of patients with multiple sclerosis, suggesting a role of this cytokine in the pathogenesis of the disease. Further, IL12 is believed to be involved in inhibition of apoptosis through the promotion of DNA repair (Schwarz, et al. (2002) “Interleukin-12 suppresses ultraviolet radiation-induced apoptosis by inducing DNA repair”, Nature Cell Biol 4:26-31).

SNP 1708460 and SNP 1708457 are located on chromosome 11 at positions 77727556 and 77724244, respectively (both intronic positions) of the GAB2 (GRB2-associated binding protein 2) gene, which is a member of the GRB2-associated binding protein (GAB) family and is similar to the GAB1 gene. The GAB2 gene encodes an adaptor molecule that is the principal activator of phosphatidylinositol-3 kinase (PIK3) in response to activation of the high affinity IgE receptor. The GAB2 protein contains a pleckstrin homology domain, proline-rich sequences, and tyrosine residues that bind to SH2 domains when they are phosphorylated (Nishida et al. (1999) “Gab-family adaptor proteins act downstream of cytokine and growth factor receptors and T- and B-cell antigen receptors”, Blood 93:1809-1816). High levels of GAB2 mRNA are detected in the heart, brain, placenta, spleen, ovary, peripheral blood leukocytes and spinal cord, and it was concluded that GAB2 may have a role in coupling cytoplasmic-nuclear signal transduction (Zhao, et al. (1999) “Gab2, a new pleckstrin homology domain-containing adapter protein, acts to uncouple signaling from ERK kinase to Elk-1”, J Biol Chem 274:19649-19654).

SNP 726185 is located on chromosome 2 at position 235130245 in an intron of the TRPM8 (transient receptor potential cation channel, subfamily member 8; aka: Trp-p8) gene is believed to be an oncogene given its high expression in melanomas (Tsavaler, et al. (2001) “Trp-p8, a novel prostate-specific gene, is up-regulated in prostate cancer and other malignancies and shares high homology with transient receptor potential calcium channel proteins”, Cancer Res 61:3760-3769). TRPM8 is a member of the “long” or melastatin subfamily of TRP channels, and is predicted to have 6-8 transmembrane domains (Peier, et al. (2002) “A TRP channel that senses cold stimuli and menthol”, Cell 108:705-715). It is specifically expressed in a subset of pain- and cold-sensing neurons. It shows high levels of expression in prostate epithelial cells and appears to be elevated in prostate cancer, as well as a number of nonprostatic primary tumors of the breast, colon, lung and skin (Tsavaler, et al. (2001) “Trp-p8, a novel prostate-specific gene, is up-regulated in prostate cancer and other malignancies and shares high homology with transient receptor potential calcium channel proteins”, Cancer Res 61:3760-3769).

SNP 532112 is located on chromosome 7 at position 142016288 in a genomic region upstream of the EPHB6 (EPH receptor B6) gene, which encodes an ephrin receptor. Ephrin receptors and their ligands, ephrins, mediate various developmental processes, particularly in the nervous system. Ephrin receptors make up the largest subgroup of the receptor tyrosine kinase (RTK) family. The ephrin receptor encoded by this gene lacks the kinase activity of most receptor tyrosine kinases (Matsuoka, et al. (1997) “Expression of a kinase-defective Eph-like receptor in the normal human brain”, Biochem Biophys Res Commun 235(3):487-492), binds to ephrin-B ligands and is strongly expressed in the brain. Increased expression of EPHB6 suppresses malignant phenotypes of neuroblastoma (Tang, et al. (2000) “Implications of EPHB6, EFNB2, and EFNB3 expressions in human neuroblastoma”, Nat Acad Sci 97:10936-10941), and also may play a role in T-cell function (Luo, et al. (2004) “EphB6-null mutation results in compromised T cell function”, J Clin Invest 114:1762-1773).

SNP 362893 is located on chromosome 6 at position 133540882 in a genomic region upstream of the EYA4 (eyes absent homolog 4 (Drosophila)) gene, which encodes a member of the eyes absent (EYA) family of proteins. The encoded protein may act as a transciptional activator and be important for continued function of the mature organ of Corti. Mutations in this gene are associated with postlingual, progressive, autosomal dominant hearing loss (Wayne, et al. (2001) “Mutations in the transcriptional activator EYA4 cause late-onset deafness at the DFNA10 locus”, Hum Molec Genet 10:195-200).

SNP 1555513 is located on chromosome 7 at position 22508917 in a genomic region upstream of the IL6 (interleukin 6 (interferon, beta 2)) gene, which encodes an immunoregulatory cytokine that activates a cell-surface signaling assembly composed of IL6, IL6RA, and the shared signaling receptor gpl3O. Constitutive expression of IL6 or its receptor may be responsible for the generation of myelomas, and the aberrant production of IL6 by neoplastic cells has been implicated as a strong contributory factor to the growth of multiple myeloma and other B-cell dyscrasias, T-cell lymphoma, renal and ovarian cell carcinomas, and Kaposi sarcoma. Variation in IL6 may also be involved in the development of osteoporosis (Ota, et al. (1999) “Linkage of interleukin 6 locus to human osteopenia by sibling pair analysis”, Hum Genet 105:253-257; Scheidt-Nave, et al. (2001) “Serum interleukin 6 is a major predictor of bone loss in women specific to the first decade past menopause”, J Clin Endocr Metab 86:2032-2042; Ota, et al. (2001) “A nucleotide variant in the promoter region of the interleukin-6 gene associated with decreased bone mineral density” J Hum Genet 46:267-272; and Chung, et al. (2003) “Association of interleukin-6 promoter variant with bone mineral density in pre-menopausal women”, J Hum Genet 48:243-248).

SNP 1128431 is located on chromosome 1 at position 162787644 in a genomic region upstream of the MGST3 (microsomal glutathione S-transferase 3) gene. The MAPEG (Membrane Associated Proteins in Eicosanoid and Glutathione metabolism) family consists of six human proteins, several of which are involved the production of leukotrienes and prostaglandin E, important mediators of inflammation. This gene encodes a microsomal enzyme which catalyzes the conjugation of leukotriene A4 and reduced glutathione to produce leukotriene C4. This enzyme also demonstrates glutathione-dependent peroxidase activity (Jakobsson, et al. (1997) “Identification and characterization of a novel microsomal enzyme with glutathione-dependent transferase and peroxidase activities”, J Biol Chem 272:22934-22939).

SNP 1611545 is located on chromosome 19 at position 45611364 in a genomic region upstream of the PRX (periaxin) gene, which encodes L- and S-periaxin, proteins of myelinating Schwann cells, and is, mutated in Dejerine-Sottas syndrome and Charcot-Marie-Tooth disease type 4F. Both L- and S-periaxin are PDZ-domain proteins required for maintenance of peripheral nerve myelin (Dytrych, et al. (1998) “Two PDZ domain proteins encoded by the murine periaxin gene are the result of alternative intron retention and are differentially targeted in Schwann cells”, J Biol Chem 273:5794-5800).

SNP 2441088 is located on chromosome 9 at position 37898658 in a genomic region upstream of the MCART1 (mitochondrial carrier triple repeat 1) gene, which encodes a mitochondrial membrane protein.

SNP 4040922 is located on chromosome 4 at position 154283058 in a genomic region upstream of the TIGD4 (tigger transposable element derived 4) gene, which encodes a protein in the tigger subfamily of the pogo superfamily of DNA-mediated transposons in humans. These proteins are related to DNA transposons found in fungi and nematodes, and more distantly to the Tc1 and mariner transposases. They are also very similar to the major mammalian centromere protein B. The exact function of this gene is not known.

SNP 1611545 is located on chromosome 19 at position 45611364 in a genomic region downstream of the SERTAD1 (SERTA domain containing 1; aka: SEI-1, TRIP-Br1) gene, which encodes a CDK4 regulator that prevents p16^{NK4a} from inhibiting the formation of cyclin D1-CDK4 complexes (Sugimoto, et al. (1999) “Regulation of CDK4 activity by a novel CDK4-binding protein, p34^{SEI-1}”, Genes Dev 13:3027-3033). CDK4 in conjunction with D-type cyclins regulate entry into the cell cycle and passage through the restriction point in late G₁ phase (Sherr, et al. (1993) “Mammalian G1 cyclins”, Cell 73:1059-1065). CDK inhibitors (CKIs) induce cell cycle arrest in response to different signals, and the INK4 family of CKIs specifically binds to and inactivates CDK4/CDK6. Thus, the SERTAD1 protein inhibits cell cycle arrest via inhibition of cyclin D1-CDK4 complexes by the CKI, p16^{INK4a}, and it accomplishes this, in part, by direct binding to CDK4 (Li, et al. (2004) “The nuclear protein p34SEI-1 regulates the kinase activity of cyclin-dependent kinase 4 in a concentration-dependent manner”, Biochemistry 43(14);4394-4399). In addition, SERTAD1 is believed to function at E2F-responsive promoters to integrate signals provided by PHD zinc finger- and/or bromodomain-containing transcription factors (Hsu, et al. (2001) “TRIP-Br: a novel family of PDH zinc finger- and bromodomain-interacting proteins that regulate the transcriptional activity of E2F-1/DP-1”, EMBO 20(9):2273-2285).

SNP 3393421 is located on chromosome 4 at position 344051 in an intron of the ZNF141 (zinc finger protein 141 (clone pHZ-44)) gene. By virtue of their role as transcriptional regulators, their abundance in the genome, and their known association with specific developmental disorders, zinc finger encoding genes are good candidates for being implicated in the multiple developmental defects associated with chromosomal aneusomy.

SNP 3492068 is located on chromosome 4 at position 72058145 in an intron of the RIPX (rap2 interaction protein x) gene, which may be involved in Ras-like GTPase signaling.

SNP 435134 is located on chromosome 20 at position 9220164 in an intron of the PLCB4 (zinc finger protein 141 (clone pHZ-44)) gene, which encodes a protein that catalyzes the formation of inositol 1,4,5-trisphosphate and diacylglycerol from phosphatidylinositol 4,5-bisphosphate. This reaction uses calcium as a cofactor and plays an important role in the intracellular transduction of many extracellular signals in the retina. Phospholipase C beta-4 is expressed in the suprachiasmatic nucleus (SCN) in the mouse. PLCB4 −/− mice have a pronounced loss of persistent circadian rhythm under constant darkness and a significantly decreased spontaneous firing rate of suprachiasmatic neurons during the subjective day (Park, et al. (2003) “Translation of clock rhythmicity into neural firing in suprachiasmatic nucleus requires mGluR-PLC-beta-4 signaling”, Nature Neurosci 6: 337-338). Further, PLCB4 is coupled to metabotropic glutamate receptors in the SCN, and this signaling pathway is involved in translating circadian oscillations of the molecular clock into rhythmic outputs of SCN neurons.

SNP 4417601 is located on chromosome 4 at position 164852476 in a genomic region downstream of the NPY5R (neuropeptide Y receptor Y5) gene, which encodes a protein that is localized in the paraventricular hypothalamic nucleus, the lateral hypothalamus, and other locations consistent with a role in the control of feeding behavior in rat brain (Gerald, et al. (1996) “A receptor subtype involved in neuropeptide-Y-induced food intake”, Nature 382:168-171).

SNP 2329698 is located on chromosome 3 at position 7487728 in an intron of the GRM7 (glutamate receptor,. metabotropic 7) gene, which encodes a protein that is expressed in many areas of the human brain, especially in the cerebral cortex, hippocampus, and cerebellum. L-glutamate is the major excitatory neurotransmitter in the central nervous system and activates both ionotropic and metabotropic glutamate receptors. Glutamatergic neurotransmission is involved in most aspects of normal brain function and can be perturbed in many neuropathologic conditions. The metabotropic glutamate receptors are a family of G protein-coupled receptors that have been divided into 3 groups on the basis of sequence homology, putative signal transduction mechanisms, and pharmacologic properties. Group III includes GRM4, GRM6, GRM7 and GRM8, and these receptors are linked to the inhibition of the cyclic AMP cascade but differ in their agonist selectivities

SNP 1367383 is located on chromosome 2 at position 166833251 in a genomic region upstream of the cancer-related GALNT3 (neuropeptide Y receptor Y5) gene, which encodes UDP-GalNAc transferase 3 (or UDP-N-acetyl-alpha-D-galactosamine:polypeptide N-acetylgalactosaminyltransferase 3), a member of the GalNAc-transferases family. This family transfers an N-acetyl galactosamine to the hydroxyl group of a serine or threonine residue in the first step of O-linked oligosaccharide biosynthesis, thereby initiating O-glycosylation of serine and threonine residues on an array of glycoproteins. Individual GalNAc-transferases have distinct activities and initiation of O-glycosylation is regulated by a repertoire of GalNAc-transferases. Although highly homologous to one another, the enzymes have different substrate specificities. Further, mutations in the GALNT3 gene are associated with familial tumoral calcinosis (Slavin, et al. (1993) “Familial tumoral calcinosis: a clinical, histopathologic, and ultrastructural study with an analysis of its calcifying process and pathogenesis”, Am J Surg Path 17:788-802; Steinherz, et al. (1985) “Elevated serum calcitriol concentrations do not fall in response to hyperphosphatemia in familial tumoral calcinosis”, Am J Dis Child 139:816-819; and Topaz, et al. (2004) “Mutations in GALNT3, encoding a protein involved in O-linked glycosylation, cause familial tumoral calcinosis”, Nature Genet 36: 579-581). The involvement of GALNT3 in PD-related disease may be related to the notion that Parkinson's disease patients may have a lower frequency of nonfatal cancers preceding Parkinson's disease, but a higher risk of cancers after the diagnosis of Parkinson's disease (Elbaz et al. Risk tables for parkinsonism and Parkinson's disease. J Clin Epidemiol 2002;55:25-31; Elbaz et al. Risk of cancer after the diagnosis of Parkinson's disease: A historical cohort study. Mov Disord (DOI 10.1002/mds.20401)).

SNP 840738 is located on chromosome 5 at position 106917999 in an intron of the EFNA5 (ephrin-A5) gene, which encodes Ephrin-A5, a member of the ephrin gene family. Ephrin-A5 prevents axon bundling in cocultures of cortical neurons with astrocytes, a model of late stage nervous system development and differentiation. The EPH and EPH-related receptors comprise the largest subfamily of receptor protein-tyrosine kinases and have been implicated in mediating developmental events, particularly in the nervous system. The ephrin-A (EFNA) class of ephrins are anchored to the membrane by a glycosylphosphatidylinositol linkage, in contrast to the ephrin-B (EFNB) class, which are transmembrane proteins. Ephrin-A5 binds to the EphB2 receptor, leading to receptor clustering, autophosphorylation, and initiation of downstream signaling. Ephrin-A5 induced EphB2-mediated growth cone collapse and neurite retraction in a model system. Further emphasized was the unexpected finding of crosstalk between A- and B-subclass Eph receptors and ephrins (Himanen, et al. (2004) “Repelling class discrimination: ephrin-A5 binds to and activates EphB2 receptor signaling”, Nature Neurosci 7: 501-509).

SNP 1276993 is located on chromosome 2 at position 80767709 in an intron of the CTNNA2 (catenin (cadherin-associated protein), alpha 2) gene. Catenins anchor cadherins to the cytoskeleton, thereby facilitating cadherin mediation of homophilic cell-cell Ca++-dependent association. Further, the CTNNA2 protein may function as a critical agent to regulate the stability of synaptic contacts (Abe, et al. (2004) “Stability of dendritic spines and synaptic contacts is controlled by alpha-N-catenin”, Nature Neurosci 7: 357-363, 2004).

SNP 1431787 is located on chromosome 2 at position 229019671 in the PARK 11 late onset Parkinson's disease susceptibility locus, which showed significant linkage to Parkinson's disease in one study but not in another (Pankratz et al., Am J Hum Genet 2003;72: 1053-1057; Prestel et al., Eur J Hum Genet 2005;13: 193-197).

SNP 3883710 is located on chromosome X at position 149463988 in a region downstream of the PASD1 (PAS domain containing 1) gene (LOC139135). X-inactivation of a major susceptibility gene could explain the gender difference in lifetime risk for Parkinson's disease (men have a 1.5 times greater risk than women) (Elbaz et al. Risk tables for parkinsonism and Parkinson's disease. J Clin Epidemiol 2002;55:25-31; Carrel and Willard, X-inactivation profile reveals extensive variability in X-linked gene expression in females. Nature 2005;434:400-404).

SNP 1032590 is located on chromosome 1 at position 54007335 in the PARK10 locus, which has been previously linked to idiopathic late onset Parkinson's disease susceptibility. The PARK10 locus was originally identified in Icelandic families via a genome-wide scan using microsatellite markers (Hicks et al. (2002) “A susceptibility gene for late-onset idiopathic Parkinson's disease”, Ann Neurol 52:549-555. This locus was subsequently shown to influence age at onset in a multi-center study of Parkinson's disease families (Li et al. (2002) “Age at onset in two common neurodegenerative diseases is genetically controlled”, Am J Hum Genet 70:985-993). To date, a specific gene within the PARK10 locus has not been identified. This SNP is within a gene designated LOC200008, which encodes a hypothetical protein with inferred oxidoreductase activity and potential involvement in cholesterol biosynthesis and electron transport. The SNP is also within 28 kb upstream of the MRPL37 (mitochondrial ribosomal protein L37) gene. Mitochondrial mechanisms of Parkinson's disease pathogenesis have been described (Dauer and Przedborski (2003) “Parkinson's disease: mechanisms and models”, Neuron 39:889-909), and a role for mitochondrial ribosomal protein genes in human disorders has been postulated (Kenmochi et al. (2001) “The human mitochondrial ribosomal protein genes: mapping of 54 genes to the chromosomes and implications for human disorders”, Genomics 77(1-2):65-70).

SNP 1747104 is located on chromosome 11 at position 106188312 in an intron of the GUCY1A2 (guanylate cyclase 1, soluble, alpha 2) gene, and SNP 928560 is located on chromosome 13 at position 50530633 in an intron of the GUCY1B2 (guanylate cyclase 1, soluble, beta 2) gene. Soluble guanylyl (or guanylate) cyclases are heterodimeric enzymes consisting of an alpha and a beta subunit, are activated by nitric oxide (NO) (Koglin, et al. (2001) J. Biol. Chem. 276(33):30737-30743; and Russwurm, et al. (2002) Mol. Cell. Biochem. 230(1-2):159-164), and catalyze the conversion of GTP to 3′, 5′-cyclic GMP and pyrophosphate. Both the GUCY1A2 and GUCY1B2 subunits are expressed in the brain (Mergia, et al. (2003) Cell Signal. 15(2):189-195; Gibb, et al. (2001) Eur. J. Neurosci. 13(3):539-544; Okamoto (2004) Int. J. Biochem. Cell Biol. 36(3):472-480; Bidmon, et al. (2004) Neurochem. Int. 45(6):821-832; and Budworth, et al. (1999) Biochem. Biophys. Res. Commun. 263(3):696-701). The NO/cGMP signaling pathway is known to play a role in neuronal development, and has been implicated in the dopaminergic fiber degeneration in the striatum that is attributed to Parkinson's disease (Chalimoniuk, et al. (2004) Biochem. Biophys. Res. Commun. 324(1):118-126). Further, some studies have shown that depletion of glutathione levels in the brain, one of the earliest biochemical alterations observed in PD brains, with participation of guanylate cyclase may change the dopamine cell-specific trophic effect of NO in the midbrain to one that is neurotoxic leading to programmed neuronal cell death (Canals, et al. (2001) J. Neurochem. 79(6):1183-1195; Canals, et al. (2003) J. Biol. Chem. 278(24):21542-21549).

The set of genes found to be associated with PD-related disease (PD-related disease genes) by the methods described herein may be used to determine or further validate cellular pathways involved in the development or pathology of PD-related disease. For example, as PD-related disease is a neurodegenerative disease, the neuronal expression or activity of a large number of PD-related disease genes may underlie their involvement in PD-related disease, including e.g. TRPM8, EPHB6, EFNA5, PRX, OR10AC1P, GRM7, NPY5R, EYA4, GAB2, CACNA1G, PRDM2, CTNNA2, SEMA5A, IL12B, GUCY12A2, GUCY1B2, and PLCB4. Similarly, genes involved in cell cycle regulation, cancer or cell death/apoptosis are also represented in the set of PD-related disease nucleic acids, e.g., SEMA5A, SHB, CARD12, CASP5, DDX31, SERTAD1, ABL1, IL12B, PRDM2, TRPM8, IL6, GUCY1A2, GUCY1B2 and GREB1. Parkinson's disease is characterized by the presence of intracellular inclusions in surviving neurons in various areas in the brain suggesting that protein metabolism or cellular trafficking may be involved in the development of the disease. Supporting this view, several PD-related disease genes are believed to be involved in such processes in the cell, e.g., PARK2, CUL3, ARFIP1, KDELR2, and SEMA5A.

The polymorphisms, alleles and associated genomic regions identified in Table 1 can be used to identify, isolate and amplify PD-related disease nucleic acids. Such nucleic acids can be used for prognostics, diagnostics, theranostics, prevention, treatment and further study of PD-related disease.

In one embodiment, nucleic acids disclosed herein that can specifically hybridize to a genomic region associated with PD-related disease, are identified in Table 1. Of course, it will be clear to one of skill that, due to the duplex nature of DNA, sequences complementary to those provided in Table 1 can also specifically hybridize to a genomic region associated with PD-related disease and are contemplated to be part of the instant invention. Nucleic acids provided herein or complementary sequences thereto can, in some embodiments, specifically hybridize to a genomic sequence having one or more polymorphisms identified in Table 1, column 5, and/or other polymorphisms in the same haplotype blocks as the polymorphisms in Table 1, column 5, or complementary sequences thereto. Methods for identifying polymorphisms in a haplotype block and in haplotype patterns within a haplotype block are provided in U.S. patent application Ser. No. 10/106,097, filed Mar. 26, 2002, entitled “Methods For Genomic Analysis”; in U.S. patent application Ser. No. 10/426,903, filed Apr. 29, 2003, entitled “Methods For Genomic Analysis”; in U.S. patent application Ser. No. 10/467,558, filed Feb. 14, 2003, entitled “Identifying SNP Patterns” (all of which are assigned to the same assignee as the present application); and in Patil, et al. (2001) “Blocks of Limited Haplotype Diversity Revealed by High-Resolution Scanning of Human Chromosome 21”, Science 294:1719-1723.

In a preferred embodiment, the nucleic acids herein are associated with resistance or susceptibility to PD-related disease. For example, a nucleic acid associated with resistance to PD-related disease is one that can specifically hybridize to a genomic region having one or more alleles identified in bold font in Table 1, column 5 and/or one or more alleles in haplotype patterns with the alleles in bold font, or complementary sequences thereto. A nucleic acid associated with susceptibility to PD-related disease is one that can specifically hybridize to a genomic region having one or more alleles underlined in Table 1, column 5 and/or one or more alleles in haplotype patterns with the underlined alleles, or complementary sequences thereto.

In certain embodiments, a set of nucleic acids is provided that can specifically hybridize to at least 2 polymorphisms, preferably at least 3 polymorphisms, at least 4 polymorphisms, at least 5 polymorphisms, at least 6 polymorphisms, at least 7 polymorphisms, at least 8 polymorphisms, or at least 9 polymorphisms associated with PD-related disease such as those identified in Table 1, and/or polymorphisms in the same haplotype blocks as the polymorphisms in Table 1, or complementary sequences thereto. In other embodiments, a set of nucleic acids is provided that can specifically hybridize to at least 2 alleles, preferably at least 3 alleles, at least 4 alleles, at least 5 alleles, at least 6 alleles, at least 7 alleles, at least 8 alleles, or at least 9 alleles associated with resistance to PD-related disease such as those identified in bold font in Table 1 column 5, and/or alleles in the same haplotype patterns as the alleles in bold font, or complementary sequences thereto. Similarly, a set of nucleic acids may be provided that can specifically hybridize to at least 2 alleles, preferably at least 3 alleles, at least 4 alleles, at least 5 alleles, at least 6 alleles, at least 7 alleles, at least 8 alleles, or at least 9 alleles associated with susceptibility to PD-related disease such as those underlined in Table 1, column 5, and/or alleles in the same haplotype patterns as the underlined alleles, or complementary sequences thereto.

A nucleic acid can be single-stranded or double-stranded. It can also be a coding (e.g., exon) or non-coding sequence (e.g., introns, 3′ or 5′ untranslated regions, and regulatory regions) or a combination of coding and non-coding nucleic acids. In a preferred embodiment, a coding PD-related disease nucleic acid is one that can specifically hybridize to at least a portion of the coding region of an associated gene, or to one or more exons of an associated gene or to one or more open reading frames of an associated gene.

A nucleic acid provided herein can be fused to at least one other nucleic acid (e.g., a tag sequence or reporter gene) to create a construct for producing a specific protein product, such as a fusion protein. A tag sequence encodes a polypeptide that can assist in isolation or purification of the protein product (e.g., glutathione-S-transferase (GST) fusion protein or a hemagglutinin A (HA) polypeptide). A reporter gene also encodes an easily assayed protein and is often used to replace other coding regions whose protein products are difficult to assay. A fusion protein is formed by the expression of a hybrid nucleic acid made by combining two nucleic acid sequences.

Conditions for nucleic acid hybridization vary depending on the buffers used, length of nucleic acids, ionic strength, temperature, etc. The term “stringent conditions” for hybridization refers to the incubation and wash conditions (e.g., conditions of temperature and buffer concentration) that permit hybridization of a first nucleic acid to a second nucleic acid. The first nucleic acid may be perfectly (e.g. 100%) complementary to the second or may share some degree of complementarity, which is less than perfect (e.g., more than 70%, 75%, 85%, or 95%). For example, certain high stringency conditions can be used which distinguish perfectly complementary nucleic acids from those less complementary, even those having only a single base mismatch. High stringency, moderate stringency and low stringency conditions for nucleic acid hybridization are known in the art. Ausubel, F. M. et al., “Current Protocols in Molecular Biology” (John Wiley & Sons 1998), pages 2.10.1-2.10.16; 6.3.1-6.3.6. The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is observed, conditions which will allow a given sequence to hybridize (e.g., selectively) with the most similar sequences in the sample can be determined. Exemplary conditions are described in Krause, et al., Methods in Enzymology, (1991) 200:546-556 and in Ausubel, et al., “Current Protocols in Molecular Biology”, (John Wiley & Sons 1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatches among the sequences that hybridize. Generally, doubling the concentration of SSC results in an increase in TM of ˜17° C. Using these guidelines, the washing temperature can be determined empirically for high, moderate or low stringency, depending on the level of mismatch sought. For example, a low stringency wash can comprise washing in a solution containing 0.2×SSC/0.1% SDS for 10 min at room temperature; a moderate stringency wash can comprise washing in a prewarmed solution (42° C.) solution containing 0.2×SSC/0.1% SDS for 15 min at 42° C.; and a high stringency wash can comprise washing in prewarmed (68° C.) solution containing 0.1×SSC/0.1 %SDS for 15 min at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid and the primer or probe used.

Furthermore, a nucleic acid is preferably isolated. Various nucleic acid isolation techniques are well known in the art, such as those described in Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997). For example, an isolated nucleic acid is one that is separated from the nucleic acids that normally flank it or from other biological materials (e.g., other nucleic acids, proteins, lipids, cellular components, etc.) in a sample.

Nucleic acids may also be amplified using polymerase chain reaction (PCR) and other techniques known in the art. See Erlich, H.A., “PCR Technology: Principles and Applications for DNA Amplification” (ed. Freeman Press, NY, N.Y., 1992); Innis M. A., et al., “PCR Protocols: A Guide to Methods and Applications” (Eds. Academic Press, San Diego, Calif., 1990). In addition to PCR, other suitable isolation and amplification methods include, for example, the ligase chain reaction (LCR) (see Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Natl. Acad. Sci. USA, 87:1874 (1990)) and nucleic acid based sequence amplification (NASBA). The latter two amplification methods involve isothermal reactions based on isothermal transcription that produces both single-stranded RNA (ssRNA) and double-stranded DNA (dsDNA) as the amplified products in a ratio of approximately 30-100 fold more ssRNA than dsDNA. Amplification methods may result in a subset of sequences being selected from a complex sample, such as those described in U.S. Ser. No. [unassigned], docket No. 100/1066-10, filed Feb. 14, 2005, entitled “Selection Probe Amplification”.

Further, homologues of the PD-related disease nucleic acids presented herein may be present in other species, and may be identified and readily isolated without undue experimentation by molecular biological techniques well known in the art using the polymorphisms, alleles and associated genomic regions identified in Table 1. Further, there may exist nucleic acids at other locations within the genome that encode proteins that have extensive homology to one or more domains of the PD-related disease polypeptides herein. These nucleic acids may be identified via similar techniques.

For example, a PD-related disease nucleic acid may be labeled and used to screen a genomic or cDNA library constructed from mRNA obtained from the organism of interest. Hybridization conditions will be of a lower stringency when the cDNA library was derived from an organism different from the type of organism from which the labeled nucleic acid was derived. Such lower stringency conditions will be well known to those of skill in the art, and will vary predictably depending on the specific organisms from which the library and the labeled nucleic acids are derived. For guidance regarding such conditions see, for example, Sambrook et al. (1989) Molecular Cloning, A Laboratory Manual, Cold Spring Harbor Press, N.Y.; and Ausubel et al. (1989) Current Protocols in Molecular Biology, Green Publishing Associates and Wiley Interscience, N.Y.

1. Probes and Primers

The nucleic acids herein can be used as probes and primers in various assays. The terms “probe” and “primer” refer to nucleic acids that hybridize, in whole or in part, in a sequence-specific manner to a complementary strand. Probes and primers include polypeptide nucleic acids, such as those described in Nielsen et al. (1991) Science 254:1497-1500.

In certain embodiments, the term “primer” refers to a single-stranded nucleic acid that can act as a point of initiation of template-directed DNA synthesis, such as in PCR. PCR reactions can be designed based on the human genome sequence and the associated genomic regions or polymorphisms identified in Table 1. For example, where a polymorphism is located in an exon, the exon can be isolated and amplified using primers that are complementary to the nucleotide sequences at both ends of the exon. Similarly, where a polymorphism is located in an intron, the entire intron can be isolated and amplified using primers that are complementary to the nucleotide sequences at both ends of the intron. Short-or long-range PCR primers may be designed to amplify the associated genomic regions or polymorphisms identified in Table 1 using methods known in the art and further described in U.S. patent applications Ser. No. 10/042,406, filed Jan. 9, 2002, entitled “Algorithms for Selection of Primer Pairs”, 10/236,480, filed Sep. 5, 2002, entitled “Methods for Amplification of Nucleic Acids”, and 10/341,832, filed Jan. 14, 2003, entitled “Apparatus and Methods for Selecting PCR Primer Pairs”.

In preferred embodiments, a probe or primer contains a region of at least about 10 contiguous nucleotides, preferably at least about 15 contiguous nucleotides, more preferably about 20 or about 30 or about 50 contiguous nucleotides, that can specifically hybridize to a complementary nucleic acid sequence. In addition, a probe or primer is preferably about 100 or fewer nucleotides, more preferably between 6 and 50 nucleotides, and more preferably between 12 and 30 nucleotides in length. In certain embodiments, a first portion of a probe or primer is perfectly complementary to a target nucleic acid, and a second portion of the probe or primer is not perfectly complementary to the target nucleic acid. In some aspects, the portion that is not perfectly complementary contains a binding site, e.g., for a polypeptide or another probe or primer.

In order to isolate, amplify and/or detect the presence of a PD-related disease nucleic acid, a probe or primer or set of such probes or primers or a combination thereof may include at least 1 polymorphism, preferably at least 2 polymorphisms, more preferably at least 3 polymorphisms, or more preferably at least 4 polymorphisms associated with PD-related disease as shown in Table 1, complementary sequences thereto, or polymorphisms that are genetically linked to the polymorphisms in Table 1 (e.g. in the same haplotype block). To isolate, amplify and/or detect the presence of a nucleic acid associated with resistance to PD-related disease, a probe or primer or set of such probe or primers may include at least 1 allele, preferably at least 2 alleles, more preferably at least 3. alleles, or more preferably at least 4 alleles associated with resistance to PD-related disease as shown in Table 1, complementary sequences thereto, or alleles that are genetically linked to the alleles in Table 1 (e.g. in the same haplotype pattern). To isolate, amplify and/or detect the presence of a nucleic acid associated with susceptibility to PD-related disease, a probe or primer or set thereof preferably includes at least 1 allele, preferably at least 2 alleles, more preferably at least 3 alleles, or more preferably at least 4 alleles associated with susceptibility to PD-related disease as shown in Table 1, complementary sequences thereto, or alleles that are genetically linked to the alleles in Table 1 (e.g. in the same haplotype pattern).

In one embodiment, a probe or primer is at least about 70% identical to at least a portion of a nucleotide sequence (or complement thereof) that is being screened for the presence of an associated genomic region, preferably at least about 80% identical, more preferably at least about 90% identical, even more preferably about 95% identical, or even 100% identical. In any embodiment, a probe or primer may be optionally labeled with, for example, a radioactive, fluorescent, biotinylated or chemiluminescent label (e.g., radioisotope, fluorescent compound, enzyme, or enzyme co-factor.) Labeled nucleic acids are useful for detection of a hybridization complex and can be used as probes for diagnostic and screening assays.

Labeled probes can be used in cloning of full-length cDNA or genomic DNA by screening cDNA or genomic DNA libraries. Classical methods of constructing cDNA libraries are taught in Sambrook et al., supra. These methods provide for the production of cDNA from mRNA and the insertion of the cDNA into viral or other expression vectors. Typically, libraries of mRNA comprising poly(A) tails can be produced with poly(T) primers. Similarly, cDNA libraries can be produced using the nucleic acids herein as primers. Libraries of cDNA can be made either from, selected tissues (e.g., normal or diseased tissue), or from tissues of a mammal treated with, for, example, a pharmaceutical agent. Alternatively, many cDNA libraries are available commercially. In a preferred embodiment, the cDNA library is made from diseased or healthy human neuronal tissues or cells. In another preferred embodiment, members of the cDNA library are larger than a nucleic acid hybridization probe, and preferably contain the whole cDNA native sequence.

Genomic DNA can be isolated in a manner similar to the isolation of full-length cDNA. Briefly, the nucleic acids herein, or fragments, derivatives or complements thereof, can be used to probe a library of genomic DNA. Preferably, a genomic DNA library is obtained from neuronal tissue or cells but this is not essential. Such libraries can be in vectors suitable for carrying large segments of a genome, such as P1 or YAC, as described in detail in Sambrook et al., 9.4-9.30. In addition, genomic sequences can be isolated from human BAC libraries, which are commercially available from Research Genetics, Inc., Huntsville, Ala., USA, for example. As an alternative, full-length cDNA, genomic DNA, or any nucleic acid, fragment, derivative or complement thereof, can be obtained by synthesis.

2. Antisense and RNAi

Antisense nucleic acids, or mimetics thereof that are complementary, in whole or in part, to one or more PD-related disease nucleic acids are provided. Antisense nucleic acids can be used in diagnostics, prognostics, theranostics and/or treatment of PD-related disease. Antisense nucleic acids hybridize under high stringency conditions to target nucleic acids (e.g., associated genomic regions or RNA derivatives thereof such as mRNA). An antisense nucleic acid can bind RNA to form a duplex or a double-stranded DNA to form a triplex, which may be assayed.

Preferably, hybridization of an antisense nucleic acid can act directly to block the translation of mRNA associated with susceptibility to PD-related disease by hybridizing to targeted mRNA and preventing protein translation. Absolute complementarity, although preferred, is not required. Antisense nucleic acids complementary to non-coding target nucleic acids associated with susceptibility to PD-related disease may also be used to inhibit translation of endogenous mRNA associated with susceptibility to PD-related disease by hybridizing to DNA regions involved in the transcription of the mRNA (e.g., regulatory regions, promoters, enhancers, etc.). While antisense nucleic acids complementary to a coding region sequence could be used, those complementary to the transcribed, untranslated region are most preferred. Antisense nucleic acids are preferably at least 10 nucleotides in length, more preferably at least 20 nucleotides, even more preferably at least 40 nucleotides in length, or more preferably at least 80 nucleotides in length. An antisense nucleic acid can be labeled for convenient detection, such as by using a radioisotope, fluorescent compound, enzyme or an enzyme co-factor.

Regardless of the choice of target sequence, it is preferred that in vitro studies be first performed to quantitate the ability of the antisense nucleic acid to inhibit mRNA expression. It is preferred that these in vitro studies utilize controls that distinguish between antisense inhibition and nonspecific biological effects of nucleic acids in a sample. Additionally, it is envisioned that results obtained using the antisense nucleic acid be compared with those obtained using a control nucleic acid. A control nucleic acid is preferably of approximately the same length as the test antisense nucleic acid and differs from the antisense nucleic acid sequence no more than is necessary to prevent specific hybridization to the target sequence.

The antisense nucleic acids herein can be modified at the base moiety, sugar moiety or phosphate backbone to improve stability of the molecule. Furthermore, the antisense nucleic acids may be hybridized or conjugated to another molecule (e.g., a peptide, hybridization triggered cross-linking agent, cleavage agent or transport agent) for targeting in a host cell or to facilitate the transport across the cell membrane (see, e.g., Letsinger et al. (1989) Proc. Natl. Acad. Sci. USA 86:6553-6556; Lemaitre et al., (1987), Proc. Natl. Acad. Sci. USA 84:648-652); for blood-brain barrier (see, e.g., PCT Publication No. W089/10134); to facilitate the hybridization-triggered cleavage agents (see, e.g., Krol et aL (1988) BioTechniques 6:958-976) or intercalating agents (see, e.g., Zon, (1988), Pharm. Res. 5:539-549).

The antisense nucleic acids may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense nucleic acid may also comprise at least one modified sugar moiety selected from the group including but not limited to arabinose, 2-fluoroarabinose, xylulose, and hexose. In yet another embodiment, the antisense nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense nucleic acid is an α-anomeric oligonucleotide. An α-anomeric oligonucleotide forms specific double-stranded hybrids with complementary RNA in which, contrary to the usual β-units, the strands run parallel to each other (Gautier, et al., (1987) Nucl. Acids Res. 15:6625-6641). The oligonucleotide is a 2′-0-methylribonucleotide (Inoue, et al., (1987) Nucl. Acids Res. 15:6131-6148), or a chimeric RNA-DNA analogue (Inoue, et al., (1987) FEBS Lett. 215:327-330).

Antisense nucleic acids (as well as other nucleic acids) herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (1988) Nucl. Acids Res. 16:3209, and methylphosphonate oligonucleotides can be prepared by use of controlled pore glass polymer supports Sarin, et al., (1988) Proc. Natl. Acad. Sci. USA 85:7448-7451, etc. Alternately, an antisense nucleic acid can be produced biologically by placing a target nucleic acid in an expression vector in an antisense orientation or by using reverse transcriptase along with other reagents to construct the complementary DNA stand.

Antisense nucleic acids should be delivered to cells that express the target nucleic acid in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies which specifically bind receptors or antigens expressed on the target cell surface) can be administered systemically.

A preferred approach to achieve intracellular concentrations of an antisense molecule sufficient to suppress translation of endogenous mRNAs utilizes a recombinant DNA construct in which the antisense oligonucleotide is placed under the control of a strong promoter (e.g., pol III or pol II). The use of such a construct to transfect target cells in a patient will result in the transcription of sufficient amounts of single-stranded RNAs which will form complementary base pairs with the endogenous sequence transcripts and thereby prevent translation of the mRNA sequence. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, (1981) Nature 290:304-310), the promoter contained in the 3′-long terminal repeat of Rous sarcoma virus (Yamamoto, et al., (1980) Cell 22:787-797), the herpes thymidine kinase promoter (Wagner, et al., (1981) Proc. Natl. Acad. Sci. USA. 78:1441-1445), and the regulatory sequences of the metallothionein gene (Brinster, et al., (1982) Nature 296:39-42). Any type of plasmid, cosmid, YAC or viral vector can be used to prepare the recombinant DNA construct that can be introduced directly into the tissue site. Alternatively, viral vectors can be used that selectively infect the desired tissue, in which case administration may be accomplished by another route (e.g., systemically).

In any of the embodiments herein, it may be necessary to compare the nucleotide sequence of the nucleic acid obtained, isolated, amplified, or cloned with that of a control. The percent identity of two nucleotide sequences can be determined, for example, by aligning the sequences for optimal comparison purposes. The nucleotides at corresponding positions are compared and the percent identity between the two sequences is a function of the number of identical positions shared by the sequences (e.g., percent identity =[(the number of identical positions/total number of positions)×100]. In some embodiments, the length of a sequence aligned for comparison purposes is at least 30%, preferably at least 40%, more preferably at least 60%, and even more preferably at least 70%, 80%, or 90% of the length of the reference sequence or a full sequence gene. An actual comparison of two nucleic acid sequences can be accomplished by well-known methods, for example, using a mathematical algorithm. In one example, such a mathematical algorithm is described in Karlin et al., (1993) Proc. Natl. Acad. Sci. USA, 90:5873-5877. In another example, such mathematical algorithm is the algorithm of Myers and Miller, (1989) CABIOS. Additional algorithms for sequence analysis are known in the art and include ADVANCE and ADAM as described in Torellis and Robotti (1994) Comput. Appl. Biosci., 10:3-5 and FASTA described in Pearson and Lipman (1988).Proc. Natl. Acad. Sci. USA, 85:2444-8.

RNAi, or “RNA interference” is a technique in which exogenous, double-stranded RNA complementary to a known target MRNA are introduced into a cell to cause the degradation of the target mRNA, thereby reducing or silencing gene expression. This method of gene regulation has been demonstrated in Drosophila, Coenorhabditis elegans, plants, and in mammalian cell cultures. In mammalian cells, siRNAs (“small-interfering RNAs” that are double-stranded) are transfected into cells. siRNAs can be created using a phage enzyme known as “DICER” and a multi-protein siRNA complex termed “RISC” (RNA induced silencing complex). Briefly, duplexes of short (˜19 nucleotides in length) RNAs with symmetric 2-nucleotide 3′-overhangs (siRNAs) are introduced into a cell where they associate with specific proteins in a ribonucleoprotein complex, which scans the MRNA in the cell and degrades the MRNA target that is homologous to the siRNA, thereby preventing translation of the mRNA message and, therefore, synthesis of the protein encoded therein. For a review of RNAi techniques, see, e.g., Huppi, et al. (2005) “Defining and Assaying RNAi in Mammalian Cells”, Molecular Cell 17(1):1-10; Grimm,et al. (2005) “Adeno-associated virus vectors for short hairpin RNA expression”, Methods Enzymol 392:381-405; Bantounas, et al. (2004) “RNA interference and the use of small interfering RNA to study gene function in mammalian systems”, J Molec Endocrin 33:545-557; Genc, et al. (2004) “RNA interference in neuroscience”, Brain Res Mol Brain Res 132(2):260-270; and Campbell, et al. (2005) “RNA interference: past, present and future”, Curr Issues Mol Biol 7(1):1-6.

3. Ribozymes, Knock-Outs and Triple Helices.

Ribozyme molecules designed to catalytically cleave target mRNA transcripts can also be used to prevent translation of such mRNA. See, e.g., PCT Publication No. WO 90/11364; Sarver, et al., (1990) Science 247: 1222-1225.

Ribozymes are enzymatic RNA molecules capable of catalyzing the specific cleavage of RNA. See Rossi, (1994) Current Biology 4:469-471. The mechanism of ribozyme action involves sequence specific hybridization of the ribozyme to complementary target RNA, followed by an endonucleolytic cleavage event. The composition of ribozyme molecules must have one or more sequences complementary to the target mRNA and must include the well known catalytic sequence responsible for mRNA cleavage. See, e.g., U.S. Pat. No. 5,093,246.

While ribozymes that cleave mRNA at site-specific recognition sequences can be used to destroy target mRNAs, the use of hammerhead ribozymes is preferred. Hammerhead ribozymes cleave mRNAs at locations dictated by flanking regions which form complementary base pairs with the target mRNA. The sole requirement is that the target MRNA have the following sequence of two bases: 5′-UG-3′. The construction and production of hammerhead ribozymes are well known in the art and are described in Myers, “Molecular Biology and Biotechnology: A Comprehensive Desk Reference,” (VCH Publishers, New York, 1995) page 833; and in Haseloff and Gerlach, (1988), Nature 334:585-591.

Preferably a ribozyme is engineered so that the cleavage recognition site is located near the 5-end of the target mRNA, i.e., to increase efficiency and minimize the intracellular accumulation of non-functional mRNA transcripts.

The ribozymes herein may further include RNA endoribonucleases, also known as “Cech-type ribozymes,” such as the one which occurs naturally in Tetrahymena thermophila (known as the IVS, or L-19 IVS RNA) and which has been extensively described in Zaug, et al., (1984) Science 224:574-578; Zaug and Cech, (1986) Science 231:470-475; Zaug, et al., (1986) Nature 324:429-433; PCT Publication No. WO 88/04300; Been and Cech, (1986) Cell 47:207-216.

As in the antisense approach, ribozymes can be composed of modified nucleic acids (e.g., for improved stability, targeting, etc.) and are preferably delivered to cells that express the target gene in vivo. A preferred method of delivery involves using a DNA construct encoding the ribozyme under the control of a strong constitutive promoter (e.g., pol III or pol II), so that transfected cells will produce sufficient quantities of the ribozyme to destroy endogenous target mRNA and inhibit translation. Because ribozymes, unlike antisense molecules, are catalytic, a lower intracellular concentration is required for efficiency.

Endogenous target gene expression can also be reduced by inactivating or “knocking out” the target nucleic acid (e.g., coding regions or regulatory regions of the target gene) using targeted homologous recombination. See Smithies, et al., (1985) Nature 317:230-234; Thomas and Capecchi, (1987) Cell 51:503-512; Thompson, et al., (1989) Cell 5:313-321. For example, a non-functional nucleic acid (or a completely unrelated DNA sequence) flanked by DNA homologous to the endogenous target nucleic acid can be used, with or without a selectable marker and/or a negative selectable marker, to transfect cells which express the target gene in vivo. Insertion of the DNA construct, via targeted homologous recombination, results in inactivation of the target gene. Such approaches can be used in humans provided the recombinant DNA constructs are directly administered or targeted to the required site in vivo using appropriate viral vectors.

Alternatively, endogenous expression of a target gene can be reduced by targeting deoxyribonucleotide sequences complementary to the regulatory region of the target gene (i.e., the target gene promoter and/or enhancers) to form triple helical structures which prevent transcription of the target gene in target cells in the body. See generally, Helene, (1991), Anticancer Drug Des., 6(6):569-584; Helene, et al., (1992), Ann. N.Y. Acad. Sci., 60:27-36; and Maher, (1992), Bioassays 14(12):807-815.

Nucleic acids to be used in triple helix formation for the inhibition of transcription should be single-stranded and composed of deoxyribonucleotides. The base composition of these oligonucleotides must be designed to promote triple helix formation via Hoogsteen base pairing rules, which generally require sizable stretches of either purines or pyrimidines to be present on one strand of a duplex. Nucleic acids may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen which are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid. Switchback nucleic acids are synthesized in an alternating 5′-3′, 3′-5′ manner, such that they base pair with first one strand of a duplex and then the other, eliminating the necessity for a sizable stretch of either purines or pyrimidines to be present on one strand of a duplex.

In instances wherein the antisense, ribozyme, “knock-out,” and/or triple helix molecules described herein are utilized to inhibit gene expression (e.g., expression of nucleic acids associated with susceptibility to PD-related disease), it is possible that the technique may so efficiently reduce or inhibit the transcription (triple helix; knock-out) and/or translation (antisense, ribozyme) of mRNA that it may cause severe negative side effects. In such cases, to ensure that substantially normal levels of target gene products or desired gene products are maintained, nucleic acids which encode polypeptides exhibiting a desired target gene activity (e.g., polypeptides associated with resistance to PD-related disease) may, be introduced into cells via gene therapy methods. The desired gene product should not contain sequences susceptible to antisense, ribozyme or triple helix treatments that are being utilized.

The antisense, ribozyme, and triple helix molecules herein may be prepared by any method known in the art for the synthesis of DNA and RNA molecules.

4. Expression Vectors and Vectors

In a preferred embodiment, the nucleic acids herein are used to over-express polypeptides associated with resistance to PD-related disease. In another preferred embodiment, the nucleic acids herein are used to underexpress polypeptides associated with susceptibility to PD-related disease. To overexpress a polypeptide, for example, a nucleic acid encoding the polypeptide of interest can be ligated to a regulatory sequence that can drive the expression of the polypeptide in the animal cell type of interest at a level that is higher than expression in the absence of such a construct. Such regulatory regions are well known to those skilled in the art. In another example, a non-coding nucleic acid (e.g., an intron or a regulatory nucleic acid) may be introduced to increase the production of a polypeptide of interest. To underexpress an endogenous polypeptide, a nucleic acid encoding a transcription factor or antisense RNA that down-regulates the polypeptide or a nucleic acid that produces, e.g., a variant or inactive polypeptide may be introduced into the genome of an animal such that the endogenous expression will be reduced or inactivated. In addition to, or in the alternative, a non-coding nucleic acid herein (e.g., an intron or a regulatory nucleic acid) may be introduced to override a native regulatory nucleic acid.

Any one or more of the nucleic acids herein can be inserted into a vector. A vector can be used, for example, to transfer nucleic acids or to express the inserted nucleic acids. An exonic associated genomic region can be in the coding region or outside the coding region. Expression vectors may be constructed using methods known in the art. Such methods include in vitro recombinant DNA techniques, synthetic techniques, in vivo genetic recombination, and other techniques described in Sambrook, J. et al. “Molecular Cloning, A Laboratory Manual,” (Cold Spring Harbor Press, Plainview, N.Y. 1989), and Ausubel, F. M. et al. “Current Protocols in Molecular Biology”, (John Wiley & Sons, New York, N.Y., 1989). A vector may also comprises one or more regulatory elements that direct the expression of a coding sequence in a host cell. Regulatory elements include but are not limited to inducible and non-inducible promoters, enhancers, operators, and other elements known to those of skill in the art that drive and regulate expression.

There are numerous types of expression vectors. One type of expression vector is a plasmid, which refers to a circular double-stranded DNA molecule into which additional DNA segments can be ligated. Another type of vector is a viral vector, wherein additional DNA segments can be ligated into a viral genome. Viral vectors include replication defective retroviruses, adenoviruses and adeno-associated viruses. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, e.g. expression vectors, are capable of directing the expression of genes to which they are operably linked. A preferable expression vector is either a plasmid, an artificial chromosome, a cosmid, or a viral vector.

The expression vectors herein can include one or more regulatory sequences, selected on the basis of the host cells to be-used and the level of expression desired. The regulatory sequences can be operably linked to the nucleic acid sequence to be expressed. The term operably linked refers to a nucleic acid of interest that is linked to one or more regulatory sequences in a manner that allows for the expression of the nucleic acid of interest. The term regulatory sequence is intended to include promoters, enhancers and other expression control elements (e.g., polyadenylation signals). Such regulatory sequences are described, for example, in Goeddel, “Gene Expression Technology: Methods in Enzymology” (1990) 185, Academic Press, San Diego, Calif. Regulatory sequences include those that direct constitutive expression of a nucleotide sequence in many types of host cells and those that direct expression of the nucleotide sequence only in certain host cells (e.g., tissue-specific regulatory sequences).

In another embodiment, a coding region of an associated genomic region can be inserted into an expression vector with or without a non-coding region of interest. The difference in expression or activity between a vector comprising both the non-coding and coding sequence can be detected using methods known in the art.

The vectors herein can be inserted into a host cell. The term “host cell” refers not only to a particular subject cell but also to the progeny or potential progeny of such a cell. Because certain modifications may occur in succeeding generations due to either mutations or environmental influences, such progeny may not, in fact, be identical cells, but are still included within the scope of the term as used herein.

Vectors can be introduced into prokaryotic or eukaryotic cells via conventional transformation or transfection techniques. For example, expression systems in bacteria include those described in Chang et al., (1978) Nature 275:615, and Siebenlist et al., (1980) Cell 20:269; expression systems in yeast include those described in Kelly and Hynes, EMBO J. (1985) 4:475-479; expression systems in insect cells include those described in Maeda et al., (1985) Nature 315:592-594 and expression in mammalian cells include those described, for example, in Dijkema et al., (1985) EMBO J. 4:761. Vector constructs can comprise either sense or antisense sequences, or both.

As used herein, the terms transformation and transfection are intended to refer to a variety of art-recognized techniques for introducing a foreign nucleic acid molecule (e.g., DNA) into a host cell, including calcium phosphate or calcium chloride co-precipitation, DEAF-dextran-mediated transfection, lipofection, or electroporation. Suitable methods for transforming or transfecting host cells can be found in Sambrook, et al. and other laboratory manuals. For stable transfection of mammalian cells, it is known that, depending upon the expression vector and transfection technique used, only a small fraction of cells may integrate the foreign DNA into their genome. In order to identify and select these integrants, a gene that encodes a selectable marker is generally introduced into the host cells along with the gene of interest. Preferred selectable markers include those that confer resistance to drugs. Nucleic acid molecules encoding a selectable marker can be introduced into a host cell on the same vector as the nucleic acids or can be introduced on a separate vector. Cells stably transfected with the introduced nucleic acid molecule can be identified by drug selection (e.g., cells that have incorporated the selectable marker gene will survive, while the other cells die).

A variety of host-expression vector systems may be utilized to express the PD-related disease coding nucleic acids of the invention. Such host expression systems represent not only the vectors by which the coding sequences may be expressed and their encoded RNAs or polypeptides purified, but also represent the cells containing these vectors. These include, but are not limited to bacteria (e.g., E. coli, B. subtilis) transformed with recombinant bacteriophage DNA, plasmid DNA or cosmid DNA expression vectors; yeast (e.g., Saccharomyces, Pichia) transformed with recombinant yeast expression vectors, insect cell systems transformed with recombinant viral expression vectors (e.g., baculovirus), plant cell systems transformed with recombinant viral expression vectors (e.g., cauliflower mosaic virus, tobacco mosaic virus) or transformed with recombinant plasmid expression vectors (e.g., Ti plasmid), and mammalian cell systems (e.g., COS, CHO, BHK, 293, 3T3 cell lines) transformed with recombinant expression constructs containing promoters derived from the genome of mammalian cells (e.g., metallothionein promoter) or from mammalian viruses (e.g., the adenovirus late promoter, vaccinia virus 7.5K promoter). Such vectors and host-expression vector systems are well known in the art and are further described in, e.g., Ruther et al. (1983), EMBO J. 2:1791; Inouye & Inouye (1985) Nucleic Acids Res. 13:3101-3109; Van Heeke & Schuster (1989) J. Biol. Chem. 264:5503-5509; Smith et al. (1983) J. Virol. 46:584; Smith, U.S. Pat. No. 4,215,051; Logan & Shenk (1984) Proc. Natl. Acad. Sci. USA 81:3655-3659; Bittner et al. (1987) Methods in Enzymol. 153:516-544; Alam (1990) Anal. Biochem. 188:245-254; MacGregor & Caskey (1989) Nucl. Acids Res. 17:2365; and Norton & Corrin (1985) Mol. Cell. Biol. 5: 281.

In addition, a host cell may be chosen that modulates the expression of a vector-encoded nucleic acid sequence, or that modifies and processes an encoded RNA or polypeptide in a specific manner. Such modifications (e.g., glycosylation, phosphorylation) and processing (e.g., cleavage, folding) of polypeptides may be important for the function of the polypeptide. Different host cells have characteristic and specific mechanisms for the post-translational modification of polypeptides. Appropriate host cells or systems can be chosen to ensure that the correct modification and processing of an encoded protein. As such, in some situations, it may be desirable to express a eukaryotic gene in a eukaryotic cell where the gene will benefit from native folding and posttranslational modifications. Such mammalian host cells include, but are not limited to CHO, VERO, BHK, HeLa, COS, MDCK, 293, 3T3, W138, etc.

Host cells can be used to produce polypeptides encoded by any of the nucleic acids herein. Suitable host cells and methods for producing polypeptides using such host cells are discussed in Goeddel, supra. For large scale protein production, a unicellular organism such as E. coli, baculovirus vectors, or cells of higher organisms such as vertebrates, particularly mammals, e.g. COS7 cells, may be useful. Host cells into which an expression vector has been introduced may be cultured in suitable medium such that the polypeptide is produced. The polypeptide herein may be isolated from the medium or from the host cell.

For long-term, high-yield production of recombinant proteins, stable expression is preferred. For example, cell lines that stably express the PD-related disease genes or polypeptides may be engineered. Rather than using expression vectors that contain viral origins of replication, host cells may be transformed with DNA controlled by appropriate expression control elements (e.g., promoter or enhancer sequences, transcription terminators, polyadenylation sites, etc.) and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci, which in turn may be cloned and expanded into cell lines. This method may advantageously be used to engineer cell lines that express the PD-related disease genes or polypeptides. Such engineered cell lines may be particularly useful in screening and evaluation of compounds that affect the endogenous activity of the PD-related disease gene or polypeptide.

A number of selection systems may be used, including but not limited to the herpes simplex virus thymidine kinase (Wigler, et al. (1977) Cell 11:223), hypoxanthine-guanine phosphoribosyltransferase (Szybalska & Szybalski (1962) Proc. Natl. Acad. Sci. USA 48:2026), and adenine phosphoribosyltransferase (Lowy, et al. (1980) Cell 22:817) genes can be employed in tk³¹, hgprt³¹ or aprt³¹ cells, respectively. Also, antimetabolite resistance can be used as the basis of selection for dhfr, which confers resistance to methotrexate (Wigler, et al. (1980) Natl. Acad. Sci. USA 77:3567; O'Hare, et al. (1981) Proc. Natl. Acad. Sci. USA 78:1527); gpt, which confers resistance to mycophenolic acid (Mulligan & Berg (1981) Proc. Natl. Acad. Sci. USA 78:2072); neo, which confers resistance to the aminoglycoside G-418 (Colberre-Garapin, et al. (1981) J. Mol. Biol. 150:1); and hygro, which confers resistance to hygromycin (Santerre, et al. (1984) Gene 30:147) genes.

An alternative fusion protein system allows for the ready. purification of non-denatured fusion proteins expressed in human cell lines (Janknecht, et aL (1991) Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

When used as a component in assay systems such as those described below, a PD-related disease polypeptide may be labeled, either directly or indirectly, to facilitate detection of a complex formed between the PD-related disease polypeptide and a candidate agent. Any of a variety of suitable labeling systems may be used including but not limited to radioisotopes such as ²²⁵¹I; enzyme labeling systems that generate a detectable calorimetric signal or light when exposed to substrate; and fluorescent labels. Where recombinant DNA technology is used to produce the PD-related disease polypeptide for such assay systems, it may be advantageous to engineer fusion proteins that can facilitate labeling, immobilization and/or detection.

Indirect labeling involves the use of a protein, such as a labeled antibody, which specifically binds to a PD-related disease polypeptide. Such antibodies include but are not limited to polyclonal, monoclonal, chimeric, single chain, Fab fragments and fragments produced by an Fab expression library.

Host cells can also be used to produce nonhuman transgenic animals. For example, in one embodiment, a host cell is a fertilized oocyte or an embryonic stem cell into which a nucleic acid (e.g., an exogenous PD-related disease gene or a nucleic acid encoding a polypeptide herein) has been introduced. Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is preferably a mammal, more preferably a rodent such as a rat or mouse, in which one or more of the cells of the animal include a transgene. In certain embodiments, a transgenic animal is a non-human animal. Other examples of transgenic animals include, for example, non-human primates, sheep, dogs, cows, goats, chickens, fish, reptiles and amphibians. A transgene is an exogenous DNA which is integrated into the genome of a cell from which a transgenic animal develops and which remains in the genome of the mature animal, thereby directing the expression of an encoded gene product in one or more cell types or tissues of the transgenic animal. As used herein, a homologous recombinant animal is preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal. In certain embodiments, a homologous recombinant animal is a non-human animal.

Methods for generating transgenic animals via embryo manipulation and microinjection, particularly animals such as mice, are conventional in the art and are described, for example, in U.S. Pat. Nos. 4,736,866, 4,870,009, 4,873,191 and in Hogan, “Manipulating the Mouse Embryo,” (Cold Spring Harbor Laboratory Press, Cold Spring Harbor, N.Y., 1986). Methods for constructing homologous recombination vectors and homologous recombinant animals are described further in Bradley (1991) Current Opinion in BioTechnology, 2:823-829. Clones of the non-human transgenic animals described herein can also be produced according to the methods described in Wilmut et al. (1997) Nature 385:810-813; and PCT Publication Nos. WO 97/07668 and WO 97/07669.

II. Polypeptides

PD-related disease polypeptides such as those regulated or encoded by the PD-related disease nucleic acids herein are useful in the prognostics, theranostics, diagnostics, prevention, treatment and study of PD-related disease. Such polypeptides may be naturally occurring or recombinantly produced using methods known in the art. In addition, PD-related disease polypeptides may include polypeptides that represent functionally equivalent polypeptides to those disclosed herein. Such a functionally equivalent polypeptide may contain deletions, additions, or substitutions of amino acid residues within the amino acid sequence encoded by PD-related disease nucleic acids herein, but that result in a “silent” change (e.g., synonymous or conservative substitution), thus producing a polypeptide that is functionally equivalent to a PD-related disease polypeptide herein.

A polypeptide may be associated with PD-related disease by virtue of one or more polymorphisms that occur within its amino acid sequence. For example, a reference polypeptide may be associated with susceptibility to PD-related disease and an alternate polypeptide may be associated with resistance to PD-related disease, or vice versa. A polypeptide may be associated with PD-related disease by virtue of one or more PD-related nucleic acids that modulate expression of the polypeptide (e.g., comprising at least a portion of a promoter, enhancer, intron, 3′ untranslated region, or other regulatory region, which may lie within or outside of the coding region for the polypeptide). For example, a reference allele within a regulatory region may cause a PD-related disease polypeptide to be expressed at a higher level than the corresponding alternate allele, or vice versa. A polypeptide may be associated with PD-related disease by virtue of a combination of one or more polymorphisms that occur within its amino acid sequence and one or more PD-related nucleic acids that modulate its expression.

A PD-related disease polypeptide can be associated with resistance or susceptibility to PD-related disease, or with a response to a treatment regimen (e.g., drug response). A PD-related disease polypeptide may be a reference polypeptide or an alternate polypeptide. A PD-related disease polypeptide may be one that is expressed at a higher or lower level in individuals having a phenotype of resistance to PD-related disease than in individuals having a phenotype of susceptibility to PD-related disease. A PD-related disease polypeptide may be one that is expressed at a higher or lower level in individuals having an efficacious response to a treatment regimen than in individuals not having an efficacious response. A PD-related disease polypeptide may be one that is expressed at a higher or lower level in individuals having an adverse response to a treatment regimen than in individuals not having an adverse response. A PD-related disease polypeptide may be one that is regulated, modulated, or encoded in whole or in part by a PD-related disease nucleic acid. In one example, a PD-related disease polypeptide can be recombinantly produced using an expression vector having a PD-related disease nucleic acid comprising a non-coding regulatory region operably linked to a PD-related disease nucleic acid encoding the PD-related disease polypeptide. The expression vector is introduced into a host cell under conditions appropriate for expression and protein synthesis. The polypeptide can then be isolated from the host cell using standard protein purification techniques.

The polypeptides of the present invention may be produced by recombinant technology using techniques well known in the art. Thus, methods for preparing the PD-related disease polypeptides by expressing PD-related disease nucleic acids are described herein. Methods which are well known to those skilled in the art can be used to construct expression vectors containing PD-related disease coding nucleic acids and appropriate regulatory elements (e.g., transcriptional/translational control signals). These methods include, for example, in vitro recombinant DNA techniques, synthetic techniques and in vivo recombination/genetic recombination. See, for example, the techniques described in Sambrook et al. (1989) Molecular Cloning, A Laboratory Manual, Cold Spring Harbor Press, N.Y.; and Ausubel et al. (eds. 1987-1993), Current Protocols in Molecular Biology, John Wiley & Sons, Inc. New York). Alternatively, RNA capable of encoding PD-related disease polypeptides may be chemically synthesized using, for example, synthesizers. See, for example, the techniques described in “Oligonucleotide Synthesis”, 1984, Gait, M. J. ed., IRL Press, Oxford.

In preferred embodiments, the polypeptides are purified. There are various degrees of purity. While a polypeptide can be purified to homogeneity, preparations in which a polypeptide is not purified to homogeneity are also useful where the polypeptide retains a desired function even in the presence of a considerable amount of other components. In some embodiments, polypeptides are substantially free of cellular material which includes preparations of a polypeptide having less than about 30% (dry weight) other polypeptides (e.g., contaminating polypeptides), less than about 20% other polypeptides, less than about 10% other polypeptides, or less than about 5% other polypeptides.

When a polypeptide is recombinantly produced, it can also be substantially free of culture medium. In preferred embodiments, culture medium represents less than about 20% of the volume of the polypeptide preparation, preferably less than about 10% of the volume of the polypeptide preparation or more preferably less than about 5% of the volume of the polypeptide preparation. Polypeptides that are substantially free of chemical precursors or other chemicals generally include those that are separated from chemicals that are involved in its synthesis. In one embodiment, the polypeptides are substantially free of chemical precursors or other chemicals such that a preparation of the polypeptides has less than about 30% (dry weight) chemical precursors or other chemicals, preferably less than about 20% chemical precursors or other chemicals, more preferably less than about 10% chemical precursors or other chemicals or more preferably less than about 5% chemical precursors or other chemicals.

As used herein, two polypeptides are substantially homologous when their amino acid sequences are at least about 45% homologous, or preferably at least about 75% homologous, or more preferably at least about 85% homologous, or even more preferably greater than about 95% homologous. To determine the percent homology of two polypeptides, the amino acid sequences are aligned for optimal comparison purposes. The amino acid residues at corresponding positions are compared. The percent homology between two amino acid sequences is a function of the number of identical positions shared by the sequences (e.g., percent homology equals (the number of identical positions/total number of positions) times 100).

Some polypeptides (e.g., those encoded by genes containing conservative polymorphisms) may have a lower degree of sequence homology but are still able to perform one or more of the same functions. For example, conservative substitutions may include substitutions of aliphatic amino acids methionine, valine, leucine and isoleucine; interchange of the hydroxyl residues serine and threonine; exchange of acidic residues aspartic and glutamic acids; substitution between amide residues asparagine and glutamine; exchange between basic residues lysine and arginine; replacements among aromatic residues phenylalanine, tyrosine and tryptophan; and substitutions between alanine and glycine.

Other polypeptides that may not be able to perform one or more of the same functions may be polypeptide variants containing one or more non-conservative substitutions, deletions, insertions or inversions of one or more amino acid residues. Amino acids that are essential for function of a polypeptide can be identified by various methods known in the art, such as site-directed mutagenesis or alanine-scanning mutagenesis. See Cunningham et al., (1989) Science, 244:1081-1085. The latter procedure can introduce a single alanine mutation at every residue in the molecule. The resulting polypeptide variants are then tested for biological activity in vitro or in vivo. Residues that are critical for polypeptide activity or inactivity are identified by comparing the two polypeptides (with and without the alanine mutation). Polypeptide activity can also be determined by structural analysis such as crystallization, nuclear magnetic resonance or photoaffinity labeling. See Smith et al, (1992) J. Mol. Biol., 224:899-904; and de Vos et al. (1992) Science, 255:306-312.

1. Fusion Proteins

Any polypeptides herein can be made part of a fusion protein. The term “fusion protein” or “fusion polypeptide” refers to a polypeptide that is operatively linked to one or more different polypeptides. A PD-related disease fusion polypeptide comprises at least one or more PD-related disease polypeptides, or fragments thereof, and may further comprise one or more polypeptides that are not associated with PD-related disease, or fragments thereof. For example, a non-PD-related disease polypeptide can be fused to the N-terminus or C-terminus of a PD-related disease polypeptide. “Operatively linked” indicates that the polypeptides are fused together such that the polypeptides maintain one or more of the functions present in the native polypeptides (i.e., wild-type functions). In a preferred embodiment, the function of a PD-related disease polypeptide in a fusion polypeptide is functional (e.g., retains its wild-type function). Examples of fusion polypeptides that may not affect the function of a polypeptide include GST-fusion polypeptides in which the PD-related disease polypeptide sequences are fused to the C-terminus of the GST sequences. Other types of fusion polypeptides include enzymatic fusion polypeptides, for example β-galactosidase fusions, yeast two-hybrid GAL fusions, poly-His fusions and Ig fusions. Fusion polypeptides, e.g., poly-His fusions, can facilitate the purification of recombinant polypeptide. In some host cells, such as mammalian cells, expression and secretion of a PD-related disease polypeptide can be increased using a heterologous signal sequence. Therefore, in a preferred embodiment, a PD-related disease polypeptide may be fused to a heterologous signal sequence at its N-terminus. In another embodiment, a fusion protein may comprise a PD-related disease polypeptide and various portions of immunoglobulin constant regions such as the Fc portion. Fc portions are useful in therapy and diagnosis and may result in improved pharmacokinetic properties. Fc portions can also be used in high-throughput screening assays to identify binding molecules, agonists and antagonists. See, e.g., Bennett et al.; J. of Molec. Recog., (1995). 8:52-58 and Johanson et al., (1995) J. of Biol. Chem., 270,16:9459-9471. In a preferred embodiment, soluble fusion proteins comprise a PD-related disease polypeptide and one or more of the constant regions of heavy or light chains of immunoglobulins (e.g. IgG, IgM, IgA, IgD, IgE).

A fusion protein can be produced by standard recombinant DNA techniques as described herein. For example, DNA fragments coding for the different polypeptide sequences may be ligated together in accordance with conventional techniques. In other aspects, the fusion gene can be synthesized by conventional techniques such as automated DNA synthesizers. Alternatively, PCR amplification of nucleic acid fragments can be carried out using anchor primers which give rise to complementary overhangs between two consecutive nucleic acid fragments that can subsequently be annealed and reamplified to generate a chimeric nucleic acid sequence. Moreover, many expression vectors are commercially available that already encode a fusion moiety (e.g., a GST protein). A nucleic acid encoding a polypeptide herein can be cloned into such an expression vector such that the fusion moiety is linked in-frame to the polypeptide.

2. Antibodies

Any of the polypeptides herein, or fragments, derivatives, or complements thereof, can be used as an immunogen (e.g., epitope) to generate polypeptide-specific antibodies. Antibodies can be used to detect, isolate and/or inhibit the activity of one or more PD-related disease polypeptides. Described herein are methods for the production of antibodies capable of specifically recognizing one or more PD-related disease epitopes. Such antibodies may include, but are not limited to polyclonal, monoclonal, humanized, chimeric, single chain antibodies, Fab fragments, F(ab′)₂ fragments, fragments produced by Fab expression library, anti-idiotypic (anti-Id) antibodies and epitope-binding fragments of any of the above.

Such antibodies may be used, for example, in the detection of a PD-related disease gene or polypeptide in a biological sample, or, alternatively, as a method for the inhibition of abnormal gene activity and/or the results thereof. Thus, such antibodies may be utilized as part of PD-related disease treatment methods, and/or may be used as part of diagnostic techniques whereby patients may be tested for abnormal levels of PD-related disease polypeptides, or for the presence of abnormal forms of such polypeptides.

To generate PD-related disease antibodies, various host animals may be immunized by injection with a PD-related disease polypeptide or a fragment thereof (“PD-related disease epitope”). Such host animals may include but are not limited to goats, rabbits, mice, rats and humans, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. In preferred embodiments, an epitope is at least about 6 amino acids, at least about 9 amino acids, at least about 20 amino acids, at least about 40 amino acids, or at least about 80 amino acids in length. The epitope or polypeptide fragment preferably comprises a domain, segment or motif that can be identified by analysis using well-known methods, for example, signal polypeptides, extracellular domains, transmembrane segments or loops, ligand binding regions, zinc finger domains, DNA binding domains, acylation sites, glycosylation sites or phosphorylation sites.

Polyclonal antibodies are heterogeneous populations of antibody molecules derived from the sera of animals immunized with an antigen, such as a PD-related disease polypeptide, or an antigenic functional derivative thereof. Polyclonal antibodies are prepared by immunizing a suitable host animal with a desired antigen, which may be supplemented with adjuvants as described above. The antibody titer in the immunized subject can be monitored over time using methods known in the art, such as by using an enzyme linked immunosorbent assay (ELISA). The antibodies can then be isolated from the subject (e.g., from blood) and further purified using techniques, such as protein A chromatography, to obtain the IgG fraction.

At an appropriate time after immunization, such as when the antibody titers are highest, antibody-producing cells can be obtained from the subject and used for the preparation of monoclonal antibodies. Monoclonal antibodies are populations of antibodies that contain only one species of an antigen-binding site and are capable of immunoreacting with only one particular epitope of a PD-related disease polypeptide. A monoclonal antibody composition, therefore, typically displays a single binding affinity for a particular polypeptide or epitope with which it immunoreacts.

There are numerous methods known in the art for producing monoclonal antibodies. In one example, monoclonal antibodies can be obtained by fusing individual lymphocytes (typically splenocytes) from an immunized animal (typically a mouse or a rat) with cells derived from an immortal B lymphocyte tumor (typically a myeloma) to produce a hybridoma. The culture supernatants of the resulting hybridoma cells are screened to identify a hybridoma producing a monoclonal antibody that specifically binds to a polypeptide of interest. (See, e.g., Kohler et al. (1975) Nature 256:495-497 and U.S. Pat. No. 4,376,110.) Other techniques for producing hybridoma include the human B cell hybridoma technique (Kozbor et al. (1983) Immunol. Today 4:72); the EBV-hybridoma technique (Cole et al. (1985) Monoclonal Antibodies and Cancer Therapy) Alan R. Liss, Inc., pp. 77-96) and the trioma techniques. Such antibodies may be of any immunoglobulin class including IgG, IgM, IgE, IgA, IgD and any subclass thereof. The hybridoma producing the mAb of this invention may be cultivated in vitro or in vivo.

Alternatively, monoclonal antibodies can be identified and isolated by screening a combinatorial immunoglobulin library, such as an antibody phage display library. The library can be screened with one or more of the polypeptides herein. Identified members are then isolated using techniques known in the art. Kits for generating and screening phage display libraries are commercially available. See for example, the Pharmacia Recombinant Phage Antibody System, Catalog No. 27-9400-01, and the Stratagene SurjZAPTM Phage Display Kit, Catalog No. 240612. Other methods and reagents for generating and screening antibody display libraries are disclosed in PCT Publication No. WO 92/01047; PCT Publication No. WO 90/02809; Fuchs et al. (1991) Bio/Technology, 9:1370-1372; Hay et al. (1992) Hum. Antibod. Hybridomas 3:81-85; Huse et al. (1989) Science 246:1275-1281; and Griffith et al. (1993) EMBO J. 12:725-734.

In addition, techniques developed for the production of “chimeric antibodies” (Morrison et al. (1984) Proc. Natl. Acad. Sci. 81:6851-6855; Neuberger et al (1984) Nature 312:604-608; Takeda et al. (1985) Nature 314:452-454) by splicing the genes from an antibody molecule of appropriate antigen specificity from one species together with genes from an antibody molecule of appropriate biological activity from another species can be used. A chimeric antibody is a molecule in which different portions are derived from different animal species, such as those having a variable region derived from a murine mAb and a human immunoglobulin constant region. For example, a monoclonal antibody may be chimeric and/or humanized. Humanized monoclonal antibodies can be obtained using standard recombinant DNA techniques in which the variable region genes (e.g., of a rodent antibody), are cloned into a mammalian expression vector containing the appropriate human light change and heavy chain region genes. In this example, the resulting chimeric monoclonal antibodies have the antigen-binding capacity from the variable region of the rodent but are significantly less immunogenic because of the humanized light and heavy chain regions. See, e.g., Surender K. Vaswani, Ann. (1998) Allergy Asthma. Immunol. 81:105-119.

Alternatively, techniques described for the production of single chain antibodies (U.S. Pat. No. 4,946,778; Bird (1988) Science 242:423-426; Huston et al. (1988) Proc. Natl. Acad. Sci. USA 85:5879-5883; and Ward et al. (1989) Nature 334:544-546) can be adapted to produce PD-related disease gene-single chain antibodies. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al. (1989) Science 246:1275-1281) to allow rapid and easy identification of monoclonal Fab fragments with the desired specificity.

Any of the antibodies can further be coupled to a substance (e.g., a label or tag) for detection of a polypeptide-antibody binding complex. Examples of labels include, enzymes, prosthetic groups, fluorescent materials, luminescent materials, bioluminescent materials, or radioactive materials. Examples of suitable enzymes include, for example, horseradish peroxidase, alkaline phosphatase, β-galactosidase, or acetylcholinesterase. Examples of suitable prosthetic group complexes include, for example, streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin. An example of a luminescent material is luminol. Examples of bioluminescent materials include luciferase, luciferin and aequorin. Examples of suitable radioactive material include ₁₂₅I, ₁₃₁I, ₃₅S or ₃H.

The antibodies can be used to isolate one or more PD-related disease polypeptides using standard techniques such as affinity chromatography or immunoprecipitation. The antibodies can also be used to detect the presence or absence of a particular polypeptide (e.g., a polypeptide associated with resistance or susceptibility to PD-related disease) in a cell, cell lysate, cell supernatant, tissue sample or elsewhere. Preferably, the antibodies can further be used to inhibit or suppress the activity of such polypeptides by specifically binding to the polypeptides.

III. Diagnostic and Prognostic Assays

The nucleic acids, polypeptides, antibodies and other compositions herein may be utilized as reagents (e.g., in pre-packaged kits) for prognosis or diagnosis of susceptibility or resistance to PD-related disease. A variety of methods may be used to prognosticate and diagnose susceptibility or resistance to PD-related disease. The following methods are provided as examples and not as limitations of means to diagnose PD-related disease.

1. Detection of PD-Related Disease Nucleic Acids

Detection of the presence or increased level of one or more nucleic acids, or fragments, derivatives, polymorphisms, variants or complements thereof, associated with resistance to PD-related disease is a prognostic and diagnostic for resistance to PD-related disease. Similarly, detection of a decreased level of one or more nucleic acids, or fragments, derivatives, polymorphisms, variants or complements thereof, associated with resistance to PD-related disease is a prognostic and diagnostic for susceptibility to PD-related disease. On the other hand, detection of the presence or increased level of one or more nucleic acids, or fragments, derivatives, polymorphisms, variants or complements thereof, associated with susceptibility to PD-related disease is a prognostic and diagnostic for susceptibility to PD-related disease. Similarly, detection of a reduced level of one or more nucleic acids, or fragments, derivatives, polymorphisms, variants or complements thereof, associated with susceptibility to PD-related disease is a prognostic and diagnostic for resistance to PD-related disease.

In some aspects of the present invention, a gene comprising, in linkage disequilibrium with, or under the control of a PD-related disease nucleic acid may be differentially expressed. “Differential expression” as used herein refers to both quantitative and qualitative differences in a gene's expression patterns including, e.g., changes in tissue-specificity or temporal aspects of expression. As such, a differentially expressed gene may be expressed at a different time or level in a cell/tissue exhibiting a PD-related disease phenotype as compared to a cell/tissue not exhibiting the PD-related disease phenotype. For example, a differentially expressed gene may have its expression increased or decreased in normal (e.g., control or unaffected) versus PD-related disease conditions (e.g., case or affected). Such a differentially expressed gene will therefore exhibit an expression pattern that is present in either control or case samples, but is not detectable in both. Detection of such expression patterns may be made by standard techniques well known to those of skill in the art, for example, differential screening (Tedder et al. (1998) Proc. Natl. Acad. Sci. USA 85:208-212), subtractive hybridization (Hedrick et al. (1984) Nature 308:149-153; Lee et al. (1984) Proc. Natl. Acad. Sci. USA 88:2825), differential display (Liang et al., U.S. Pat. No. 5,262,311), reverse transcriptase- (RT-) PCR and/or Northern analysis. Detection of a differential expression pattern of one or more nucleic acids, or fragments, derivatives, polymorphisms, variants or complements thereof, associated with susceptibility to PD-related disease is a prognostic and diagnostic for susceptibility to PD-related disease; likewise, detection of a differential expression pattern of one or more nucleic acids, or fragments, derivatives, polymorphisms, variants or complements thereof, associated with resistance to PD-related disease is a prognostic and diagnostic for resistance to PD-related disease.

In other aspect of the present invention, a gene comprising, in linkage disequilibrium with, or under the control of a PD-related disease nucleic acid may exhibit differential allelic expression. “Differential allelic expression” as used herein refers to both qualitative and quantitative differences in the allelic expression of multiple alleles of a single gene present in a cell. As such, a gene displaying differential allelic expression may have one allele expressed at a different time or level as compared to a second allele in the same cell/tissue. For example, an allele associated with PD-related disease may be expressed at a higher or lower level than an allele that is not associated with PD-related disease, even though both are alleles of the same gene and are present in the same cell/tissue. Differential allelic expression and analysis methods therefore are disclosed in detail in U.S. patent application Ser. No. 10/438,184, filed May 13, 2003 and U.S. patent application Ser. No. 10/845,316, filed May 12, 2004, both of which are entitled “Allele-specific expression patterns”. Detection of a differential allelic expression pattern of one or more nucleic acids, or fragments, derivatives, polymorphisms, variants or complements thereof, associated with susceptibility to PD-related disease is a prognostic and diagnostic for susceptibility to PD-related disease; likewise, detection of a differential allelic expression pattern of one or more nucleic acids, or fragments, derivatives, polymorphisms, variants or complements thereof, associated with resistance to PD-related disease is a prognostic and diagnostic for resistance to PD-related disease.

Detection of nucleic acids may be made using any method known in the art, for example, Southern or northern analyses, in situ hybridization analyses, single-stranded conformational polymorphism analyses, polymerase chain reaction analyses and nucleic acid microarray analyses, all of which are well known to those of skill in the art. Such analyses may reveal not only the alleles present in a test sample, but also both quantitative and qualitative aspects of the expression pattern of PD-related disease polypeptides. In particular, such analyses may reveal expression patterns of polypeptides associated with resistance or susceptibility to PD-related disease.

In one example, a diagnosis or prognosis is made using a test sample containing genomic DNA or RNA obtained from an individual to be tested. The individual can be an adult, child or fetus. In a preferred embodiment, the individual is a human. The test sample can be from any source which contains genomic DNA or RNA, including for example, blood, amniotic fluid, cerebrospinal fluid, skin, muscle, buccal or conjunctival mucosa, placenta, gastrointestinal tract or other tissues. In a preferred embodiment a DNA or RNA sample is obtained from neuronal tissue. Alternatively, a test sample of DNA from fetal cells or tissue can be obtained by appropriate methods such as by amniocentesis or chorionic villus sampling. The test sample is subjected to one or more tests to identify the presence or absence of a nucleic acid of interest (e.g., a PD-related disease nucleic acid).

In one embodiment, the test sample is subjected to purification, isolation and/or amplification techniques, many of which are well known in the art (e.g., Sambrook et al. (1989) Molecular Cloning, A Laboratory Manual, Cold Spring Harbor Press, N.Y.; and Ausubel et al. (eds. 1987-1993), Current Protocols in Molecular Biology, John Wiley & Sons, Inc. New York).

In one embodiment, Southern blot, northern blot or similar analyses methods are used to identify the presence or absence of one or more genomic DNA sequences associated with resistance or susceptibility to PD-related disease using complementary nucleic acid probes. In certain embodiments, the nucleic acid probes are labeled before they are contacted with the test sample; in other embodiments, the nucleic acids in the test sample are labeled before they are contacted with the nucleic acid probes.

In hybridization analysis, a solution containing both the test sample and the nucleic acid probes is maintained under conditions sufficient to allow for specific hybridization of the nucleic acid probes to specific nucleic acids in the test sample (“target nucleic acids”). In a preferred embodiment, a nucleic acid probe and a target nucleic acid specifically hybridize with no mismatches. In some aspects, a nucleic acid in a test sample may comprise both a target nucleic acid that is complementary to a nucleic acid probe and a region that is not complementary to a nucleic acid probe. For example, in certain embodiments, a portion of a test sample nucleic acid that is not complementary to a nucleic acid probe may contain a binding site for a primer (e.g., PCR primer). Specific hybridization can be performed under stringent conditions disclosed herein and can be detected using standard methods. Hybridization is indicative of the presence or absence of a nucleic acid in the test sample that is complementary to a nucleic acid probe. Specific hybridization of a target nucleic acid to a nucleic acid probe that comprises or is complementary to a nucleic acid associated with resistance to PD-related disease is a diagnostic or prognostic for resistance to PD-related disease. Specific hybridization of a target nucleic acid to a nucleic acid probe that comprises or is complementary to a nucleic acid associated with susceptibility to PD-related disease is a diagnostic or prognostic for susceptibility to PD-related disease. More than one nucleic acid probe can be used simultaneously (e.g., in a microarray).

In a preferred embodiment, a nucleic acid probe is an allele-specific probe. See Saild, R. et al., (1986) Nature 324:163-166. An allele-specific probe is a nucleic acid, nucleic acid mimetic, or a combination thereof, of approximately 10-50 base pairs or more preferably approximately 15-30 base pairs that specifically hybridizes to one or more target nucleic acids comprising an allele complementary to the allele-specific probe. An allele-specific nucleic acid can be prepared using standard methods. Allele-specific probes can be used to identify the presence or absence of one or more alleles of a polymorphism in a test sample obtained from an individual. A target nucleic acid is amplified using any method known in the art and/or described herein. Flanking sequences may also be amplified. In the case of Southern analysis, the amplified target nucleic acid is dot-blotted, using standard methods and the blot is then contacted with an allele-specific nucleic acid probe. See Ausubel, F. el al., “Current Protocols in Molecular Biology” (eds. John Wiley & Sons). Detection of specific hybridization of an allele-specific probe to a target nucleic acid comprising an allele associated with resistance to PD-related disease is a diagnostic or prognostic for resistance to PD-related disease. Detection of specific hybridization of an allele-specific probe to a target nucleic acid comprising an allele associated with susceptibility to PD-related disease is a diagnostic or prognostic for susceptibility to PD-related disease.

In one example, a target nucleic acid is a nucleic acid associated with resistance to PD-related disease (e.g., comprises at least one allele or haplotype pattern associated with resistance to PD-related disease). Nucleic acid probes or sets or kits thereof (whether for Southern analysis, or other nucleic acid analysis techniques) may include one or more alleles associated with resistance to PD-related disease, more preferably two or more alleles associated with resistance to PD-related disease, more preferably three or more alleles associated with resistance to PD-related disease or more preferably four or more alleles associated with resistance to PD-related disease.

In another example, a target nucleic acid is a nucleic acid associated with susceptibility to PD-related disease (e.g., comprises at least one allele or haplotype pattern associated with susceptibility to PD-related disease). Nucleic acid probes or sets or kits thereof (whether for Southern analysis, or other nucleic acid analysis techniques) may include one or more alleles associated with susceptibility to PD-related disease, more preferably two or more alleles associated with susceptibility to PD-related disease, more preferably three or more alleles associated with susceptibility to PD-related disease, or more preferably four or more alleles associated with susceptibility to PD-related disease.

One method for detecting PD-related disease nucleic acids is northern analysis. Northern analysis can be used to identify gene expression patterns (e.g., levels of mRNA expression in different cell types or tissues, or during different developmental stages) of PD-related disease nucleic acids. See Ausubel, F. el al., “Current Protocols in Molecular Biology” (eds. John Wiley & Sons 1999). For northern analysis, a test sample of RNA is obtained from an individual by appropriate means. Specific hybridization of the test sample of RNA to a nucleic acid probe that is complementary to an RNA sequence associated with resistance to PD-related disease (e.g., encoding a polypeptide associated with resistance to PD-related disease) is a diagnostic or prognostic for resistance to PD-related disease. Specific hybridization of the test sample of RNA to a nucleic acid probe that is complementary to an RNA sequence associated with susceptibility to PD-related disease (e.g., encoding a polypeptide associated with susceptibility to PD-related disease) is a diagnostic or prognostic for susceptibility to PD-related disease. A nucleic acid probe is preferably labeled for northern blot analysis. A nucleic acid probe is preferably an allele-specific probe complementary to one or more of the polymorphisms described in Table 1, or may include kits or collections of probes with more than one of such probes.

Alternative diagnostic and prognostic methods employ amplification of target nucleic acids associated with resistance or susceptibility to PD-related disease, e.g., by PCR. This is especially useful for target nucleic acids present in very low quantities. In one embodiment, amplification of target nucleic acids associated with resistance to PD-related disease indicates their presence and is a prognostic and diagnostic of resistance to PD-related disease. In a related embodiment, amplification of target nucleic acids associated with susceptibility to PD-related disease indicates their presence and is a prognostic and diagnostic of susceptibility to PD-related disease.

In another embodiment, cDNA is obtained from test sample RNA nucleic acids by reverse transcription. Nucleic acid sequences within the cDNA may be used as templates for amplification reactions. For detection of amplified products, the nucleic acid amplification may be performed using labeled primers or labeled nucleotides. Alternatively, enough amplified product may be made such that the product may be visualized by standard ethidium bromide staining or by utilizing other suitable nucleic acid staining methods. Alternatively, the amplified product may be labeled subsequent to the amplification reaction by methods well known to those of ordinary skill in the art (e.g., end-labeling).

The above-described methods for determining expression patterns of PD-related disease genes may also be performed on an isolated cell population of a particular cell type derived from a given tissue. Additionally, in situ hybridization techniques may be utilized to provide information regarding which cells within a given tissue express a PD-related disease nucleic acid. Such analyses may provide information regarding a specific biological function of a PD-related disease nucleic acid, and any genes or genomic regions in linkage equilibrium therewith.

Microarrays can also be utilized for diagnosis and prognosis of resistance or susceptibility to PD-related disease. Microarrays comprise probes that are complementary to target nucleic acid sequences from an individual. A microarray probe is preferably allele-specific. In one embodiment, the microarray comprises a plurality of different probes, each coupled to a surface of a substrate in different known locations and each, capable of binding complementary strands. See, e.g.,. U.S. Pat. No. 5,143,854 and PCT Publication Nos. WO 90/15070 and WO 92/10092. These microarrays can generally be produced using mechanical synthesis methods or light-directed synthesis methods that incorporate a combination of photolithographic methods and solid phase oligonucleotide synthesis methods. See Fodor et al., (1991) Science 251:767-777; and U.S. Pat. No. 5,424,186. Techniques for the mechanical synthesis of microarrays are described in, for example, U.S. Pat. No. 5,384,261.

Once a microarray is prepared, one or more target nucleic acids are hybridized to the microarray before the microarray is scanned. Typical hybridization and scanning procedures are described in PCT Publication Nos. WO 92/10092 and WO 95/11995, and U.S. Pat. No. 5,424,186. Briefly, target nucleic acid sequences that include one or more previously identified polymorphisms are amplified and labeled by well-known techniques, such as attachment of a fluorescent moiety or using labeled primers during amplification (e.g. PCR). Primers that are complementary to both strands of the target sequence (one primer complementary to one strand upstream and the other primer complementary to the other strand downstream from a polymorphism) may be used to amplify the target region. Asymmetric PCR techniques may be used. An amplified target, preferably incorporating a label, is then hybridized with the microarray under appropriate conditions. Upon completion of hybridization and washing of the microarray, the microarray is scanned to determine the position on the microarray to which the target sequence hybridizes. The hybridization data obtained from the scan is typically in the form of fluorescence intensities as a function of location on the microarray.

Although primarily described in terms of a single detection block, such as for the detection of a single polymorphism, microarrays can include multiple detection blocks, and thus be capable of analyzing multiple specific polymorphisms. In an alternative arrangement, detection blocks may be grouped within a single microarray or in multiple separate nucroarrays so that varying optimal conditions may be used during the hybridization of the target to the microarray. For example, it may be desirable to provide for the detection of polymorphisms that fall within G-C rich stretches of a genomic sequence separately from those that fall in A-T rich segments for optimization of hybridization conditions. Additional description of use of nucleic acid microarrays for detection of polymorphisms can be found, for example, in U.S. Pat. Nos. 5,858,659 and 5,837,832, the entire teachings of which are incorporated by reference herein.

Other methods to detect polymorphic nucleic acids include, for example, direct manual sequencing (Church and Gilbert, (1988) Proc. Natl. Acad. Sci. USA 81:1991-1995; Sanger, F. et al. (1977) Proc. Natl. Acad. Sci. USA 74:5463-5467; and U.S. Pat. No. 5,288,644); automated fluorescent sequencing; single-stranded conformation polymorphism assays; clamped denaturing gel electrophoresis; denaturing gradient gel electrophoresis (Sheffield, V.C. et al. (1981) Proc. Natl. Acad. Sci. USA 86:232-236), mobility shift analysis (Orita, M. et al. (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); heteroduplex analysis; Tm-shift genotyping (Germer et al. (1999) Genome Research 9:72-78); kinetic PCR (Germer et al. (2000) Genome Research 10:258-266); chemical mismatch cleavage (Cotton et al. (1985) Proc. Natl. Acad. Sci. USA 85:4397-4401); RNase protection assays (Myers, R.M. et al. (1985) Science 230:1242); and use of polypeptides which recognize nucleotide mismatches, such as E. coli mutS protein.

2. Detection of PD-Related Disease Polypeptides

Detecting the presence, level of expression, activity and location of PD-related disease polypeptides may be used as a diagnostic or prognostic for resistance or susceptibility to PD-related disease. Briefly, detection of the presence, level of expression or enhanced activity of polypeptides associated with resistance to PD-related disease is a diagnostic and prognostic for resistance to PD-related disease. Detection of the presence, level of expression or enhanced activity of polypeptides associated with susceptibility to PD-related disease is a diagnostic and prognostic for susceptibility to PD-related disease.

Proteins may be analyzed from any tissue or cell type, but preferably neuronal tissues may be used. Analyses can be made in vivo or in vitro. In a preferred embodiment a biopsy (or tissue sample) is obtained from brain tissue (e.g., from the basal ganglia, or more specifically from the striate body, or more specifically from the substantia nigra) of an individual to be tested.

Methods to detect and isolate polypeptides are known to those of skill in the art and include, for example, enzymes linked immunosorbent assays (ELISAs), immunoprecipitations, immunofluorescence, immunoblotting, Western blotting, spectroscopy, colorimetry, electrophoresis and isoelectric focusing. See U.S. Pat. No. 4,376,110; see also Ausubel, F. et al., “Current Protocols in Molecular Biology” (Eds. John Wiley & Sons, chapter 10). Protein detection and isolation methods employed may also be those described in Harlow and Lane (Harlow, E. and Lane, D., “Antibodies: A Laboratory Manual,” Cold Spring Harbor Laboratory Press, Cold Spring Harbor, N.Y., 1998).

In one embodiment, the presence, amount and location of polypeptides associated with resistance to PD-related disease can be determined using a probe or an antibody that specifically binds one or more polypeptides associated with resistance to PD-related disease. In another embodiment, the presence, absence, amount or location of a polypeptide associated with susceptibility to PD-related disease can be determined using a probe or antibody that specifically bind one or more polypeptides associated with susceptibility to PD-related disease.

Antibodies, such as those described herein may be used to determine the presence of a polypeptide associated with resistance or susceptibility to PD-related disease.

In a preferred embodiment, a probe or antibody is labeled directly or indirectly. Direct labeling involves coupling (physically linking) a detectable substance to an antibody or a probe. Indirect labeling involves the reactivity of the probe with another reagent that is directly labeled. Examples of indirect labeling include detection of a primary antibody using a fluorescently labeled secondary antibody, and end-labeling of a DNA probe with biotin such that it can be detected with fluorescently labeled streptavidin.

A solid support may be utilized to immobilize either the antibody or probe or the sample (e.g., PD-related disease polypeptide). In one example, a sample may be immobilized onto a solid support such as nitrocellulose, which is capable of immobilizing cells, cell particles, or soluble proteins. The support may then be washed with suitable buffers followed by treatment with a detectably labeled antibody. The amount of bound labeled antibody on the solid support may then be detected by conventional means. Well known supports include glass, polystyrene, polypropylene, polyethylene, dextran, nylon, amylases, natural and modified celluloses, polyacrylamides, gabbros, and magnetite.

The antibodies herein can be linked to an enzyme and used in an enzyme immunoassay. See Voller, “The Enzyme Linked Immunosorbent Assay (ELISA)”, Diagnostic Horizons 2:1-7 (Microbiological Associates Quarterly Publication, Walkersville, Md. 1978); Maggio, “Enzyme Immunoassay” (CRC Press, Boca Raton, Fla. 1980); Ishikawa, et al., “Enzyme Immunoassay” (Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes that can be used to label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Detection can be accomplished by calorimetric methods which employ a chromogenic substrate for the enzyme. Detection can also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect wild type or mutant peptides through the use of a radioimmunoassay. See Weintraub, B., “Principles of Radioimmunoassays, Seventh Training Course on Radioligand Assay Techniques” (The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wavelength, its presence can be detected by measuring emitted fluorescence. Among the most commonly used fluorescent labeling compounds are fluorescein isothiocyanate, rhodamine, phycoerythrin, phycocyanin, allophycocyanin, o-phthaldehyde and fluorescamine. The fluorescently labeled antibody can be coupled with light microscopic, flow cytometric or fluorimetric detection. In one example, antibodies or fragments thereof may be employed histologically, as in immunofluorescence or immunoelectron microscopy, for in situ detection of a polypeptide associated with resistance or susceptibility to PD-related disease. In situ detection may be accomplished by removing a histological specimen from a patient, such as by biopsy. The specimen is then contacted with a labeled antibody described herein. The antibody or fragment is preferably contacted by overlaying the labeled antibody or fragment onto the sample. This procedure allows for the determination of the presence, absence, amount and location of a polypeptide of interest.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of-particularly useful chemiluminescent labeling compounds are luminol, isoluminol, theromatic acridinium ester, imidazole, acridinium salt and oxalate ester.

Likewise, a bioluminescent compound may be used to label the antibodies herein. Bioluminescence is a type of chemiluminescence found in biological systems in which a catalytic protein increases the efficiency of the chemiluminescent reaction. The presence of a bioluminescent protein is determined by detecting the presence of luminescence. Preferred bioluminescent compounds for purposes of labeling antibodies are luciferin, luciferase and aequorin.

In one embodiment, the presence (or absence) of a polypeptide associated with PD-related disease in a sample (e.g., a cell, cell lysate, tissue, whether in vivo or in vitro) can be established by contacting the sample with an antibody and then detecting a binding complex. The presence of a polypeptide associated with resistance to PD-related disease is a diagnostic and prognostic of resistance to PD-related disease. The presence of a polypeptide associated with susceptibility to PD-related disease is a prognosis and diagnosis of susceptibility to PD-related disease.

In another embodiment, the level of expression or sequence of a polypeptide associated with PD-related disease in a test sample is compared with the level of expression or sequence of the same polypeptide in a control sample. A control sample may have a known level of expression of the polypeptide, and/or can be a sample from a healthy individual or from a different tissue or organ from the test individual.

Alterations in the level of expression or sequence of a PD-related disease polypeptide may be indicative of susceptibility or resistance to PD-related disease. In one example, a test sample from an individual is assessed for a change in expression (e.g., level of transcription or translation) and/or sequence (e.g., splicing variants, polymorphisms) of a polypeptide associated with susceptibility to PD-related disease. Detection of an increased level of expression of a polypeptide associated with susceptibility to PD-related disease may be a prognosis or diagnosis of, for example, an early onset of PD-related disease or an increased susceptibility to PD-related disease. On the contrary, detection of a reduced level of a polypeptide associated with susceptibility to PD-related disease may be indicative of, for example, a reduced susceptibility to PD-related disease or an effective treatment against PD-related disease (e.g., if the test sample is from an individual after treatment and the control sample is from the same individual before treatment). Detection of an increased level of a polypeptide associated with resistance to PD-related disease may be a prognosis or diagnosis of, for example, increased immunity to PD-related disease or an effective treatment regimen against PD-related disease. On the other hand, detection of a reduced level of a polypeptide associated with resistance to PD-related disease may be a prognosis or diagnosis of, for example, decreased immunity to PD-related disease or an ineffective treatment regimen against PD-related disease. Similarly, detection of an increase in compositions (including, e.g., peptides, derivatives, variants, splicing variants) associated with susceptibility to PD-related disease may be a prognosis or diagnosis of an earlier onset or more severe symptoms of PD-related disease, while detection of an increase in compositions associated with resistance to PD-related disease may be a prognosis or diagnosis for immunity or reduced risk for developing PD-related disease.

Further, it may be useful to compare the level of expression of a reference PD-related disease polypeptide to the level of expression of an. alternate or variant PD-related disease polypeptide in a cell or tissue that is heterozygous for a nonsynonymous polymorphism in a coding region. Such a cell or tissue may be expected to produce equivalent amounts of both the reference and alternate polypeptides encoded by the coding region. However, if measurement of the amounts of these two polypeptides indicates that one is produced at a statistically higher level than the other, then this is an indication that there is another regulatory mechanism at play. For example, it may be in indication that the coding region is exhibiting differential allelic expression, expressing one allele at a higher level than the other; that the RNA from one allele is being processed differently than the RNA for the other allele (e.g., via degradation, splicing, translation, etc.); or that the reference polypeptide is being processed differently than the alternate polypeptide (e.g., via degradation, post-translational modification, etc.)

Kits useful in diagnosis and prognosis include reagents comprising, for example, instructions for use and analysis; means for collecting a tissue or cell sample; nucleic acid probes or primers (e.g., for amplification, reverse transcriptase and detection); labels (e.g., for nucleic acids or proteins); microarrays, gels, membranes or other detection apparati; restriction enzymes (e.g., for RFLP analysis); allele-specific. probes; antisense nucleic acids; antibodies; and other protein binding probes, any of which may be labeled.

IV. Screening Assays and Agents

The following assays may be used to identify agents that modulate the nucleic acids and/or polypeptides associated with PD-related disease. Such modulation may be direct or indirect, and may include, for example, changes in expression, activity or function. Such agents may, for example, interact directly or indirectly with PD-related disease genes or regulatory sequences thereof e.g. to up- or down-regulate expression; interact directly or indirectly with PD-related disease RNAs in the nucleus or cytoplasm of cells (e.g., during or after transcription; before, during, or after splicing; or before or during translation); interact directly or indirectly with PD-related disease polypeptides e.g. during or after translation, or before or after post-translational modification; or interact with molecules that bind PD-related disease nucleic acids or PD-related disease polypeptides (“binding molecule”) to result in an alteration in PD-related disease nucleic acid or PD-related disease polypeptide expression and/or activity.

Examples of agents include, but are not limited to: transcription factors, binding molecules, antisense nucleic acids, PNAs, mimetics, small or large organic or inorganic molecules, polypeptides (e.g., soluble peptides or Ig-tailed fusion peptides), antibodies (e.g., monoclonal, polyclonal, humanized, anti-idiotypic, chimeric or single chain antibodies, Fab, F(ab′)₂, Fab expression library fragments, and epitope-binding fragments thereof), fusion proteins, prodrugs, drugs in trials, previously approved drugs, drugs developed for indications other than PD-related disease, and any fragments, derivatives, variants or complements of any of the above. Such agents can be used separately or in combination.

Agents identified via these assays may be utilized to prevent, treat, diagnose and prognosticate PD-related disease. For example, whereby PD-related disease results from an overall lower level of RNAs or polypeptides associated with resistance to PD-related disease, agents that enhance or stimulate the expression or activity of such RNAs or polypeptides may be used to treat or prevent PD-related disease. In another example, whereby PD-related disease results from an over all higher level of RNAs or polypeptides associated with susceptibility to PD-related disease, agents that inhibit or diminish the expression or activity of such RNAs or polypeptides may be used to treat or prevent PD-related disease. In yet another example, a single agent or a combination of agents may be used that modulate both polypeptides or nucleic acids associated with resistance to PD-related disease and polypeptides or nucleic acids associated with susceptibility to PD-related disease.

1. Screening Assays For Agents that Modulate the Expression of Coding Nucleic Acids

In one embodiment, agents that modulate (enhance, inhibit, delay, alter the timing of, or otherwise change) the level of expression of a PD-related disease coding nucleic acid can be identified by comparing the level of expression of such coding nucleic acid in the presence of a test agent and in a control. A modulation of expression may occur at the DNA level (e.g., a transcription factor, etc.) or at the RNA level (antisense RNA, splicing, RNA-binding protein, etc.) A control can be in the absence of the test agent or a previously established level of expression. A solution or sample (e.g., cell or tissue culture) containing nucleic acids encoding a PD-related disease polypeptide can be contacted with a test agent. A solution can comprise, for example, cells or cell lysates containing the PD-related disease gene as well as other elements necessary for transcription/translation. Cells not suspended in solution as well as animal models may also be used.

If the level of expression of the PD-related disease coding nucleic acid (e.g., PD-related disease gene) is greater by an amount that is statistically significant from the level of expression in the control, then the test agent is an agonist of PD-related disease coding nucleic acid expression or activity. If the level of expression in the presence of the test agent is less by an amount that is statistically significant from the level of expression in the control, then the test agent is an antagonist of the expression of PD-related disease coding nucleic acids. The level of expression of coding nucleic acids can be evaluated, for example, by determining the level of mRNA or polypeptides that are expressed, and/or any other method herein or known in the art, including but not limited to Northern analysis, Western blotting and antibodies.

In addition, complexes of nucleic acid and protein agents may be detected by methods well known in the art. For example, such methods may utilize chromatography, microarrays, fluorescent labeling, and other methods further described in the section entitled “Immobilization Assays” herein.

In some embodiments, an agent is an agonist to the expression of PD-related disease genes associated with resistance to PD-related disease or an antagonist to the expression of PD-related disease genes associated with susceptibility to PD-related disease. In other embodiments, an agent is both an agonist to the expression of PD-related disease genes associated with resistance to PD-related disease and an antagonist of PD-related disease genes associated with susceptibility to PD-related disease. In a preferred embodiment, the agent increases the resistance and/or decreases the susceptibility of an organism (e.g., human) to PD-related disease by modulating the expression of one or more PD-related disease genes.

2. Screening Assays For Agents that Modulate the Expression of Coding Nucleic Acids by Interacting With Regulatory Regions

In another embodiment, agents that modulate the expression of PD-related disease coding nucleic acids by interacting with a PD-related disease regulatory region (e.g., enhancers, introns, 5′ and 3′ untranslated regions (e.g., promoters) and uORF's) are provided. For example, agents that modulate transcription or translation of nucleic acids herein (e.g., transcription factors) can be identified by contacting a solution containing non-coding nucleic acids associated with PD-related disease operably linked to a reporter gene with a test agent. After contact with the test agent, the level of expression of the reporter gene (e.g., the level of mRNA or polypeptide expressed) is assessed and compared with the level of expression in a control (e.g., the level of expression in the absence of a test agent or a level of expression that has previously been established). If the level of expression in the test sample is greater than the level of expression in the control sample by a statistically significant amount, then the test agent is an agonist of expression. If the level of expression in the test sample is less than the level of expression in a control sample by a statistically significant amount, then the test agent is an antagonist of the expression.

In some embodiments, an agent is an antagonist to the expression of PD-related disease genes associated with susceptibility to PD-related disease. In other embodiments, an agent is an agonist to the expression of PD-related disease genes associated with resistance to PD-related disease. Preferably, an agent is both an antagonist to the expression of PD-related disease genes associated with susceptibility to PD-related disease and an agonist to the expression of PD-related disease genes associated with resistance to PD-related disease. In a preferred embodiment, the agent increases the resistance and/or decreases the susceptibility of an organism (e.g., human) to PD-related disease by interacting with one or more PD-related disease regulatory nucleic acids.

3. Screening Assays For-Agents that Enhance/Inhibit Polypeptide Activity

In another embodiment, agents that modulate (enhance, inhibit or otherwise alter) the activity of polypeptides associated with PD-related disease (e.g., enhance the presence of certain splicing variants, or modulate one or more functions of the polypeptide (e.g., binding activity)) are identified by contacting a test agent with a cell, cell lysate or a solution containing nucleic acids and/or polypeptides associated with PD-related disease and comparing the activity of the polypeptides with their activity in a control (in absence of the test agent or a previously established level activity). If the activity of polypeptides associated with PD-related disease is enhanced by an amount that is statistically significant from the level of activity of the same polypeptides in a control, then the agent is an agonist of the activity of such polypeptides. If the activity of polypeptides associated with PD-related disease is inhibited by an amount that is statistically significant from the level of activity of the same polypeptides in a control, then the agent is an antagonist of the activity of such polypeptides. The activity of PD-related disease polypeptides may be modulated, e.g., by enhancing or inhibiting the expression of such polypeptides (i.e., increasing or decreasing the production of the polypeptides); by enhancing or inhibiting the activity of one or more such polypeptides (e.g., by altering the enzyme kinetics, binding affinity, etc. of the polypeptides); or by changing the cellular localization of one or more of such polypeptides.

In some embodiments, an agent is an agonist of the activity of polypeptides associated with resistance to PD-related disease. In other embodiments, an agent is an antagonist of the activity of polypeptides associated with susceptibility to PD-related disease. Preferably, an agent is both an agonist of the activity of polypeptides associated with resistance to PD-related disease and an antagonist of the activity of polypeptides associated with susceptibility to PD-related disease. In a preferred embodiment, the agent increases the resistance and/or decreases the susceptibility of an organism (e.g., human) to PD-related disease by modulating the activity of one or more PD-related disease polypeptides.

4. Screening Assays For Protein Agents that Bind PD-Related Disease Polypeptides

In another embodiment, assays can be used to identify protein agents that interact or bind one or more of the polypeptides herein, e.g., a PD-related disease polypeptide. Any method suitable for detecting protein-protein interactions may be employed for identifying protein agents that interact with or bind to PD-related disease polypeptides. Among the traditional methods that may be employed are co-immunoprecipitation, crosslinking, and co-purification through gradients or chromatographic columns.

In one embodiment, a yeast two-hybrid system, such as that described by Fields and Song (Fields, S. and Song, O., (1989) Nature 340:245-246), can be used to identify polypeptides that interact with one or more PD-related disease polypeptides. A yeast- two-hybrid system employs two vectors. The first vector has a DNA binding domain; the second, a transcription activation domain. Each domain is fused to a sequence encoding a different polypeptide. If the polypeptides interact with one another, transcriptional activation can be achieved, and transcription of specific markers can be used to identify the presence of interaction and transcriptional activation. In one example, a first vector contains a nucleic acid encoding a DNA binding domain and a PD-related disease polypeptide, and a second vector contains a nucleic acid encoding a transcription activation domain and test polypeptide which may potentially interact with the PD-related disease polypeptide (e.g., a binding agent). Incubation of yeast containing the first vector and the second vector under appropriate conditions (e.g., mating conditions such as those used in the Matchmaker system from Clontech (Palo Alto, Calif.)) allows for the identification of colonies that express the markers of interest. These colonies can be examined to identify the polypeptide(s) that interact with the PD-related disease polypeptide tested. The binding molecules may be use as agents to alter the activity or expression of a PD-related disease polypeptide as described above.

In another embodiment, a protein microchip may be used to identify polypeptides that bind to PD-related disease polypeptides or any other polypeptide herein. A protein microchip or microarray is provided having one or more protein complexes and/or antibodies selectively immunoreactive with a polypeptide of interest. Protein microarrays are becoming increasingly important in both proteomics research and protein-based detection and diagnosis of diseases. The protein microarrays in accordance with this embodiment are be useful in a variety of applications including, e.g., large-scale or high-throughput screening for compounds capable of binding to the protein complexes or modulating the interactions between the interacting protein members in the protein complexes.

Protein microarrays can be prepared in a number of methods known in the art. An example of a suitable method is that disclosed in MacBeath and Schreiber, (2002) Science, 289:1760-1763. Essentially, glass microscope slides are treated with an aldehyde-containing silane reagent (SuperAldehyde Substrates purchased from TeleChem International, Cupertino, Calif.). Nanoliter volumes of protein samples in a phosphate-buffered saline with 40% glycerol are then spotted onto the treated slides using a high-precision contact-printing robot. After incubation, the slides are immersed in a bovine serum albumin (BSA)-containing buffer to quench the unreacted aldehydes and to form a BSA layer that functions to prevent non-specific protein binding in subsequent applications of the microchip. Alternatively, as disclosed in MacBeath and Schreiber, proteins or protein complexes of the present invention can be attached to a BSA-NHS slide by covalent linkages. BSA-NHS slides are fabricated by first attaching a molecular layer of BSA to the surface of glass slides and then activating the BSA with N,N′-disuccinimidyl carbonate. As a result, the amino groups of the lysine, aspartate, and glutamate residues on the BSA are activated and can form covalent urea or amide linkages with protein samples spotted on the slides. See MacBeath and Schreiber, Science, 289:1760-1763 (2000).

Another example of a useful method for preparing a protein: microchip is disclosed in PCT Publication Nos. WO 00/4389A2 and WO 00/04382. First, a substrate or chip base is covered with one or more layers of thin organic film to eliminate any surface defects, insulate proteins from the base materials, and to ensure uniform protein array. Next, a plurality of protein-capturing agents (e.g., antibodies, peptides, etc.) are arrayed and attached to the base that is covered with the thin film. Proteins or protein complexes can then be bound to the capturing agents forming a protein microarray. The protein microchips are kept in flow chambers with an aqueous solution.

The protein microarrays herein can also be made by the method disclosed in PCT Publication No. WO 99/36576, which is incorporated herein by reference. For example, a three-dimensional hydrophilic polymer matrix, i.e., a gel, is first dispensed on a solid substrate such as a glass slide. The polymer matrix gel is capable of expanding or contracting and contains a coupling reagent that reacts with amine groups. Thus, proteins and protein complexes can be contacted with the matrix gel in an expanded aqueous and porous state to allow reactions between the amine groups on the protein or protein complexes with the coupling reagents thus immobilizing the proteins and protein complexes on the substrate. Thereafter, the gel is contracted to embed the attached proteins and protein complexes in the matrix gel.

The protein microchips of the present invention can also be prepared with other methods known in the art, e.g., those disclosed in U.S. Pat. Nos. 6,087,102, 6,139,831, 6,087,103; PCT Publication Nos. WO 99/60156, WO 99/39210, WO 00/54046, WO 00/53625, WO 99/51773, WO 99/35289, WO 97/42507, WO 01/01142, WO 00/63694, WO 00/61806, WO 99/61148, WO 99/40434, all of which are incorporated herein by reference.

5. Screening Assays for Agents that Interfere with PD-Related Disease Polypeptide Interaction with Binding Agents

The polypeptides herein may interact in vivo with one or more cellular or extracellular binding agents (e.g., polypeptides, nucleic acids, etc.) to form a complex. Agents that disrupt such an interaction may be used to regulate the activity or function of the PD-related disease polypeptides herein. Such agent may include, but are not limited to molecules such as antibodies, peptides, and the like. Assays that assess the impact of a test agent on the activity of a PD-related disease polypeptide in relation to a cellular or extracellular binding agent are provided. These assays involve the preparation of a reaction mixture containing a PD-related disease polypeptide and a cellular or extracellular binding agent and a time sufficient to allow the two products to interact and bind thus forming a complex.

A PD-related disease polypeptide may be, for example, an entire protein or a fragment thereof. For example, a PD-related disease polypeptide may correspond to the binding domain of a PD-related disease polypeptide. Likewise, a binding agent may be a fragment of a full-length binding agent. Any number of methods routinely practiced in the art can be used to identify and isolate the protein's binding site. These methods include, but are not limited to, mutagenesis of one of the genes encoding the proteins and screening for disruption of binding in a co-immunoprecipitation assay. Compensating mutations in the gene can be selected. Sequence analysis of the genes encoding the respective proteins will reveal the mutations that correspond to the region of the protein involved in interactive binding. Alternatively, one protein can be anchored to a solid surface, and allowed to interact with and bind-to-its labeled binding partner, which has been treated with a proteolytic enzyme, such as trypsin. After washing, a short, labeled peptide comprising the binding domain may remain associated with the solid material, which can be isolated and identified by amino acid sequencing. Also, once a gene coding for a protein is obtained, short gene segments can be engineered to express peptide fragments of the protein, which can then be tested for binding activity and purified or synthesized.

To test an agent for inhibitory activity, reaction mixtures are prepared in the presence and absence of the test agent. The test agent can be initially included in the reaction mixture or added at a time subsequent to the addition of the PD-related disease polypeptide and/or its cellular or extracellular binding agent. Control reaction mixtures can be incubated without the test agent or with a placebo agent. Formation of complexes between PD-related disease polypeptides and cellular or extracellular binding agents is measured in both the control and test reaction mixtures. A difference in the formation of a complex in the control reaction and the test reaction mixture indicates that the compound affects the interaction of the PD-related disease polypeptide and the cellular or extracellular binding agent. For example, the agent may enhance or inhibit binding between the PD-related disease polypeptide and the binding agent. Additionally, complex formation in a reaction mixture containing a test agent and a first PD-related disease polypeptide may be compared to complex formation in a reaction mixture containing the test agent and a second PD-related disease polypeptide that is encoded by a different nucleic acid sequence than the first PD-related disease polypeptide. In certain embodiments, the first and second PD-related disease polypeptides are encoded by different alleles of the same gene. This comparison can be important in those cases in which it is desirable to identify agents that disrupt interaction of a particular reference or variant PD-related disease polypeptide.

The screening assays for agents that interfere with PD-related disease polypeptide interaction with binding agents may be conducted in a heterogeneous or homogeneous format. Heterogeneous assays involve anchoring one of the binding partners onto a solid phase and detecting complexes anchored on the solid phase at the end of the reaction. In homogeneous assays, the entire reaction is carried out in a liquid phase. In either approach, the order of addition of reactants can be varied to obtain different information about the agents being tested. In either example, test agents that affect the interaction between the PD-related disease polypeptides and the cellular or extracellular binding agents can be tested, for example, by competition by adding the test agent to the reaction mixture prior to, post, or simultaneously within the PD-related disease polypeptide and cellular or extracellular binding agents and assessing the difference in complex formation. Alternatively, test agents that disrupt or otherwise affect formed complexes, (e.g., compounds (e.g., with higher binding constants) that displace one of the components from the complex) can be tested by adding the test agent to the reaction mixture after the complexes have been formed.

In a heterogeneous assay system, either the PD-related disease polypeptide or the binding agent is anchored onto a solid surface, and its binding partner (either the binding agent or the PD-related disease polypeptide, respectively), which is not anchored, is labeled, either directly or indirectly. The anchored species may be immobilized by covalent or non-covalent attachments. Non-covalent attachment may be accomplished by coating the solid surface with a solution of the species to be anchored to the surface and drying. Alternatively, an immobilized antibody specific for the species may be used to anchor it to the solid surface. The surfaces may be prepared and stored for future use. In some embodiments, microtiter plates are utilized.

In order to conduct the assay, the binding partner of the anchored species is exposed to the coated surface with or without the test agent. After the reaction is complete, unreacted components are removed (e.g., by washing) and any complexes formed will remain immobilized on the solid surface. The detection of complexes anchored on the solid surface can be accomplished in a number of ways. Where the binding partner was pre-labeled, the detection of label immobilized on the surface indicates that complexes were formed. Where the binding partner is not pre-labeled, an indirect label can be used to detect complexes anchored on the surface; e.g., using a labeled antibody specific for the binding partner (the antibody, in turn, may be directly labeled or indirectly labeled with a labeled anti-Ig antibody). Depending on the order of addition of reaction components, test compounds that inhibit complex formation or that disrupt preformed complexes can be detected.

Alternatively, the reaction can be conducted in a liquid phase in the presence or absence of the test agent, the reaction products separated from unreacted components, and complexes detected; e.g., using an immobilized antibody specific for one binding partner to anchor any complexes formed in solution, and a labeled antibody specific for the other binding partner to detect anchored complexes. Again, depending on the order of addition of reactants to the liquid phase, test compounds that inhibit complex or that disrupt preformed complexes can be identified.

In an alternate embodiment of the invention, a homogeneous assay can be used. In this approach, a preformed complex of a PD-related disease polypeptide and the interactive cellular or extracellular protein is prepared in which one of the binding partners is labeled, but the signal generated by the label is quenched due to complex formation (see, e.g., U.S. Pat. No. 4,109,496 by Rubenstein which utilizes this approach for immunoassays). The addition of a test agent that competes with and displaces one of the binding partners from the preformed complex will result in the generation of a signal above background. In this way, test agents which disrupt PD-related disease polypeptide-cellular or extracellular protein interaction can be identified.

The ability or effectiveness of a test agent to bind to a PD-related disease polypeptide, a cellular or extracellular binding agent, or a complex thereof can be assessed, for example, by coupling a test agent with a radioisotope label such that binding of the test agent to the PD-related disease polypeptide, binding agent, or complex can be determined by detecting the label (e.g., ¹²⁵I, ³⁵S, ¹⁴C, or ³H) either directly or indirectly (e.g., by direct counting of radioemission or by scintillation counting). Alternatively, test agents can be enzymatically labeled with, for example, horseradish peroxidase, alkaline phosphatase or luciferase and the enzymatic label can be detected by determination of conversion of an appropriate substrate to a product.

In another embodiment, the ability of a test agent to interact with a PD-related disease polypeptide, binding agent, or complex thereof can be assessed without the labeling of any of the interactants. For example, a microphysiometer can be used to detect the interaction of a test agent with a PD-related disease polypeptide or a binding agent without the labeling of either the test agent, the PD-related disease polypeptide or the binding agent. See McConnell, H.M. et al. (1992) Science 257:1906-1912. As used herein, a “microphysiometer” (e.g., Cytosensor (Molecular Devices, Sunnyvale, Calif.)) is an analytical instrument that measures the rate at which a cell acidifies its environment using a light-addressable potentiometric sensor (LAPS). Changes in this acidification rate can be used as an -indicator of the interaction between the binding agent and the PD-related disease polypeptide.

6. Screening for Small Molecules

Agents that modulate the expression, function and/or activity of PD-related disease nucleic acids or polypeptides can be obtained using any of the numerous approaches in combinatorial library methods known in the art, including: biological libraries; natural products libraries; spatially addressable parallel solid phase or solution phase libraries; synthetic library methods requiring deconvolution; the ‘one-bead one-compound’ library method; and synthetic library methods using affinity chromatography selection. The biological library approach is largely limited to polypeptide libraries, while the other four approaches are applicable to polypeptide, non-peptide oligomer or small molecule libraries of compounds. See Lam, K.S. (1997) Anticancer Drug Des. 12:145.

Non-peptide agents or small molecules are generally preferred because they are more readily absorbed after oral administration and have fewer potential antigenic determinants. Small molecules are also more likely to cross the blood brain barrier than larger protein-based pharmaceuticals. Methods for screening small molecule libraries for candidate protein-binding molecules are well known in the art and may be employed to identify molecules that modulate (e.g., through direct or indirect interaction) one or more of the PD-related disease polypeptides herein. Briefly, PD-related disease polypeptides may be immobilized on a substrate and a solution including the small molecules is contacted with the PD-related disease polypeptide under conditions that are permissive for binding. The substrate is then washed with a solution that substantially reflects physiological conditions to remove unbound or weakly bound small molecules. A second wash may then elute those compounds that are bound strongly to the immobilized polypeptide. Alternatively, the small molecules can be immobilized and a solution of PD-related disease polypeptides can be contacted with the column, filter or other substrate on which the small molecules are immobilized. The ability to detect binding of a PD-related disease polypeptide to a small molecule may be facilitated by labeling (e.g., radio-labeling or chemiluminescence) the polypeptide or small molecule.

In another embodiment, electronic molecular modeling applies an algorithm to screen small molecule databases for ligands and molecules that interact or bind with PD-related disease polypeptides or those in pathways therewith. See Meng et al., (1992) J. Comp. Chem. 15:505. In one example the DOCK3.5 is used to screen for small molecules that interact with PD-related disease polypeptides, preferably the binding pocket of a PD-related disease polypeptide. A “negative image” of the binding pocket on a protein surface is created. The image is created by the computational equivalent of placing atom-sized spheres into the binding pocket. A representative set of spheres are identified by DOCK3.5 that fit extremely well into the binding pocket. The generated spheres constitute an irregular grid that is matched to the atomic centers of potential ligands. The list of atom centers, or more conveniently the matrix of interatomic distances linking these atom centers forms a useful description of the binding site. The matrix of interatomic distances for the putative ligand is also made. The best mutual overlap of the two matrices is sought. This alignment specifies the orientation of the ligand relative to the negative image of the protein and thus docks the ligand into the protein's binding pocket.

Non-peptide agents or small molecule libraries can be prepared by a synthetic approach, but recent advances in biosynthetic methods using enzymes may enable one to prepare chemical libraries that are otherwise difficult to synthesize chemically. Small molecule libraries can also be obtained from various commercial entities, for example, SPECS and BioSPEC B.V. (Rijswijk, the Netherlands), Chembridge Corporation (San Diego, Calif.), Comgenex USA Inc., (Princeton, N.J.), Maybridge Chemical Ltd. (Cornwall, U.K.), and Asinex (Moscow, Russia). These small molecule libraries can be screening in a high throughput manner to identify one or more agents. For example, a high throughput screening assay for small molecules that was disclosed in Stockwell, B.R. et al., Chem. & Bio., (1999) 6:71-83, is a miniaturized cell-based assay for monitoring biosynthetic processes such as DNA synthesis and post-translational processes.

7. Immobilization Assays

In any embodiment herein, it may be desirable to immobilize either the PD-related disease polypeptides, the test agent or other components of the assay (e.g., binding agents) on a substrate in order to facilitate the separation of bound polypeptides from unbound polypeptides, as well as to accommodate automation of the assay. A substrate can be any vessel suitable for containing the reactants. Examples of substrates include: microtiter plates, test tubes, and micro-centrifuge tubes. In one example, agents that bind a polypeptide of interest can be detected by anchoring either the polypeptide of interest (e.g., any polypeptide herein) or the test agent (e.g., antibody) to a substrate (e.g., microtiter plates) and then detecting complexes of the polypeptide of interest and test agent anchored to the substrate at the end of the reaction. Where the polypeptide of interest is anchored and the test agent is not anchored, the test agent can be labeled, either directly or indirectly. In other embodiments, the polypeptide or other components of the assay may be labeled, either directly or indirectly.

In a preferred embodiment, microtiter plates are used as the solid phase, and the anchored component can be immobilized by non-covalent or covalent attachments. Non-covalent attachments can be achieved by simply coating the solid surface with a solution of the protein and drying. In another preferred embodiment, an immobilized antibody (preferably a monoclonal antibody) specific for the polypeptide to be immobilized can be used to anchor the polypeptide to the solid surface. The surface can be prepared in advance and stored.

In another embodiment, a fusion protein (e.g., a glutathione-S-transferase fusion protein) can be provided which adds a domain that allows the polypeptides, binding agents or test agents to be bound to a matrix or other solid support. A non-immobilized component is then added to the coated surface containing the anchored component. After the reaction is complete, unreacted components are removed (e.g., by washing) and complexes anchored on the solid surface are detected. Where the non-immobilized component is pre-labeled, the detection of label immobilized on the surface indicates that the complexes were formed. Where a non-immobilized component is not pre-labeled, an indirect label can be used to detect complexes anchored on the surface, such as by using a labeled antibody specific for the non-immobilized component. The antibody can then be labeled or indirectly labeled, e.g., with an anti-Ig antibody.

Alternatively, this reaction can be conducted in a liquid phase, the reaction products separated from unreacted components, and complexes detected using, for example, an immobilized antibody specific for a polypeptide of interest or test agent to anchor the complexes formed in solution and a labeled antibody specific for the other component of the possible complex to detect anchored complexes.

In another embodiment, an assay performed in liquid phase has the pre-formed complexes of the PD-related disease polypeptides and the cellular or extracellular binding agents prepared such that either the polypeptide or the binding agents are labeled, but the signal generated from the label is eliminated or diminished due to complex formation. The addition of a test agent that competes with and displaces one of the species from the pre-formed complex results in the generation of a signal above background.

In one particular embodiment, the PD-related disease polypeptide is prepared using recombinant DNA techniques described herein and is fused to a glutathione-S-transferase (GST) gene using a fusion factor such as pGEX-5X-1, such that its activity is maintained in the resulting fusion product. The cellular or extracellular binding agent is purified and used to raise a monoclonal antibody, using methods routinely practiced in the art. This antibody can be labeled with the radioactive isotope ¹²⁵I, for example by methods known in the art. In a substrate binding assay, the GST-PD-related disease polypeptide fusion product is anchored, for example, to glutathione-agarose beads. The cellular or extracellular binding agent is then added in the presence or absence of the test agent in a manner that allows interaction and binding to occur. At the end of the reaction period, unbound material is washed away, and the labeled monoclonal antibody can be added to the system and allowed to bind to the complexed components. The interaction between the PD-related disease polypeptide and the cellular or extracellular binding agent is detected by measuring the amount of radioactivity that remains associated with the beads. A successful inhibition of the interaction by the test agent will result in a decrease in measured radioactivity.

Alternatively, the GST-bound PD-related disease polypeptide fusion product and the interactive cellular or extracellular binding agent can be mixed together in liquid in the absence of the solid glutathione-agarose beads. The test agent is added either during or after the binding agent is allowed to interact with the GST-fusion polypeptide. This mixture is then added to the glutathione-agarose beads and unbound material is washed away. The extent of inhibition of the binding agent interaction can be detected by adding the labeled antibody and comparing the radioactivity associated with the beads to that of a control reaction (e.g., lacking test agent).

The same techniques can also be employed using polypeptide fragments, derivatives, or variants that correspond to the binding domains of either the PD-related disease polypeptides or the cellular or extracellular binding agents, or both. Binding sites can be identified and isolated using any one of a number of methods known in the art, including for example site-directed mutagenesis.

Alternatively, a PD-related disease polypeptide can be anchored to a solid substrate using methods disclosed herein and allowed to interact with and bind its labeled binding agent, which has been previously treated with a proteolytic enzyme (e.g., trypsin). After washing, a short-labeled peptide comprising the binding domain remains associated with the solid material, which can be isolated and identified by amino acid sequencing. Also, once the gene coding for the cellular or extracellular binding agent is obtained, short gene segment can be engineered to express binding fragments, which can then be tested for binding activity, purified and/or synthesized.

8. Agents that Enhance/Inhibit Genes in the PD-Related Disease Pathways

PD-related disease may further be prevented or treated by administering to a patient an agent that enhances or inhibits the expression or activity of genes in the associated gene pathways. Genes in the associated gene pathways are those that act upstream or downstream of the associated genomic regions in a PD-related disease pathway, and whose gene products may interact with, bind to, compete with, induce, enhance, or inhibit, directly or indirectly, the activity, expression or function of genes in the associated genomic regions.

9. Potential Agents and Binding Sites

Agents that modulate the expression or activity of PD-related disease polypeptides include: nucleic acids, transcription factors, antisense nucleic acids, polypeptides, fusion proteins, PNAs, mimetics (e.g., soluble peptides or Ig-tailed fusion peptides), antibodies (e.g., monoclonal, polyclonal, humanized, anti-idiotypic, chimeric or single-chain antibodies, Fab, F(ab′)₂, Fab expression library fragments, and epitope-binding fragments thereof), binding molecules, prodrugs, drugs in trials, previously approved drugs, drugs developed for indications other than PD-related disease, small and large organic or inorganic molecules, and any fragments, derivatives, variants or complements of any of the above. Such agents may be used separately or in combination.

Any of the agents herein can also serve as “lead agents” in the design and development of new pharmaceuticals. For example, sequential modification of small molecules (e.g., amino acid residue replacement with peptides, functional group replacement with peptide or non-peptide compounds) is a standard approach in the pharmaceutical industry for the development of new pharmaceuticals. Such development generally proceeds from a lead agent, which is shown to have at least some of the activity of the desired pharmaceutical. In particular, when one or more agents having at least some activity of interest are identified, structural comparison of the molecules can greatly inform the skilled practitioner by suggesting portions of the lead agents that should be conserved and portions that may be varied in the design of new candidate compounds. This embodiment also encompasses means of identifying lead agents that may be sequentially modified to produce new candidate agents for use in the treatment of PD-related disease. These new agents may be tested for therapeutic efficacy (e.g., in the cell-based or animal models described herein). This procedure may be iterated until compounds having the desired therapeutic activity .and/or efficacy are identified.

10. Cell Based Assays and Animal Models

The agents herein can be tested for their ability to prevent, ameliorate or treat symptoms associated with PD-related disease, using cell-based system assays, animal models and/or clinical trials. Described herein are cell- and animal-based systems which act as models for PD-related disease. These systems may be used in a variety .of applications. For example, the cell- and animal-based model systems may be used to further characterize PD-related disease genes. Second, such assays may be utilized as part of screening strategies designed to identify agents which are capable of ameliorating PD-related disease symptoms. Thus, the animal- and cell-based models may be used to identify drugs, pharmaceuticals, therapies and interventions which may be effective in treating PD-related disease. In addition, such animal models may be used to determine the LD₅₀ and the ED₅₀ in animal subjects, and such data can be used to determine the in vivo efficacy of potential PD-related disease treatments.

Animal models can be used to determine, for example, toxicity, efficacy and/or mechanism of action of the agents identified herein. Animals of any species, including, but not limited to, mice, rats, rabbits, guinea pigs, pigs, micro-pigs, goats, and non-human primates, e.g., baboons, monkeys, and chimpanzees may be used to generate PD-related disease animal models.

Animal models for PD-related disease include both non-recombinant and recombinant (or engineered) transgenic animals. Non-recombinant animal models for PD-related disease may include, for example, genetic models. Such genetic PD-related disease models may include, for example, weaver mice, a mouse model of PD associated with homozygosity for a mutation in the H54 region of Girk2. Other animal models for PD-related disease have been previously described, e.g., in Clarke, et al. (2000) “A one-hit model of cell death in inherited neuronal degenerations”, Nature 406:195-199. Non-recombinant, non-genetic animal models of PD-related disease may include, for example, dog, pig, rabbit, rat or mouse models in which the animal has been exposed to either chemical or mechanical stressors that are associated with the development of PD-related disease. For example, murine models can be created by administering to an animal an effective amount of an agent (e.g., dopamine receptor blocker, dopamine depleter, lithium, methanol, carbon monoxide, cyanide, etc.) to elicit a response or symptom associated with PD-related disease (e.g., bradykinesia, rigidity, tremor, etc.) Such animal models can then be exposed to an agent suspected of ameliorating PD-related disease.

Additionally, recombinant animal models exhibiting phenotypic states of PD-related disease or resistance thereto can be engineered, for example, by introducing nucleic acids associated with susceptibility or resistance, respectively. In one embodiment, an engineered sequence includes at least part of the target nucleic acid sequence and disrupts the endogenous target sequence upon integration of the engineered target gene sequence into the animal's genome. Techniques for making a transgenic animal are known in the art. For example,.target gene sequences may be introduced into, and overexpressed in, the genome of the animal of interest, or, if endogenous PD-related disease gene sequences are present, they may either be overexpressed or, alternatively, be disrupted in order to underexpress or inactivate PD-related disease gene expression, such as described for the disruption of apoE in mice (Plump et al. (1992) Cell 71: 343-353). Other techniques include, for example, pronuclear microinjection disclosed in U.S. Pat. No. 4,873,191; retrovirus mediated gene transfer into germ-lines disclosed in Van der Putten et al., (1985) Proc. Natl. Acad. Sci. USA, 82:6148-6152; gene targeting in embryonic stem cells disclosed in Thomson et al., (1989) Cell 56:313-321; electroporation of embryos disclosed in Lo, (1983) Mol. Cell. Biol. (3) 1803-1814; and sperm-mediated gene transfer disclosed in Lavitrano et al, (1989) Cell 57:717-723; etc. For a review of such techniques, see Gordon (1989) Transgenic Animals, Intl. Rev. Cytol. 115:171-229.

In order to overexpress a PD-related disease gene sequence, the coding portion of the gene sequence may be ligated to a regulatory sequence which is capable of driving gene expression in the animal and cell type of interest. Such regulatory regions will be well known to those of skill in the art, and may be utilized in the absence of undue experimentation. For underexpression of an endogenous PD-related disease gene sequence, such a sequence may be isolated and engineered such that when reintroduced into the genome of the animal of interest, the endogenous gene alleles will be inactivated. Preferably, the engineered PD-related disease gene sequence is introduced via gene targeting such that the endogenous sequence is disrupted upon integration of the engineered gene sequence into the animal's genome.

The transgene may be integrated as a single transgene or in concatamers, e.g., head-to-head tandems or head-to-tail tandems. When it is desired that the PD-related disease gene transgene be integrated into the chromosomal site of the endogenous PD-related gene, gene targeting is preferred. Briefly, when such a technique is to be utilized, vectors containing some nucleotide sequences homologous to the endogenous PD-related gene of interest are designed for the purpose of integrating, via homologous recombination with chromosomal sequences, into and disrupting the function of the nucleotide sequence of the endogenous PD-related gene.

The present invention provides for transgenic animals that carry the transgene in all their cells, as well as animals which carry the transgene in some, but not all their cells, i.e., mosaic animals. The transgene may also be selectively introduced into and activated in a particular cell type by following, for example, the teaching of Lasko et al. (Lasko, M. et al. (1992) Proc. Natl. Acad. Sci. USA 89: 6232-6236). The regulatory sequences required for such a cell-type specific activation will depend upon the particular cell type of interest, and will be apparent to those of skill in the art. The transgene may also be selectively introduced into a particular cell type, thus inactivating the endogenous gene of interest in only that cell type, by following, for example, the teaching of Gu et al. (Gu, et al. (1994) Science 265: 103-106). The regulatory sequences required for such a cell-type specific inactivation will depend upon the particular cell type of interest, and will be apparent to those of skill in the art.

Once transgenic animals have been generated, the expression of the recombinant PD-related gene and polypeptide may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include but are not limited to northern blot analysis of tissue samples obtained from the animal, in situ hybridization analysis, and RT-PCR. Samples of PD-related gene-expressing tissue, may also be evaluated immunocytochemically using antibodies specific for the PD-related gene transgene gene product of interest.

The PD-related gene transgenic animals that express PD-related gene mRNA or PD-related gene transgene polypeptide (detected immunocytochemically, using antibodies directed against the PD-related polypeptide's epitopes) at easily detectable levels should then be further evaluated to identify those animals which display characteristic PD-related disease symptoms.

Additionally, specific cell types within the transgenic animals may be analyzed and assayed for cellular phenotypes characteristic of PD-related disease. Such cellular phenotypes may include a particular cell type's pattern of expression as compared to known expression profiles of the particular cell type in animals exhibiting PD-related disease symptoms. Such transgenic animals serve as suitable model systems for PD-related disease.

Once target gene transgenic founder animals are produced, they may be bred, inbred, outbred, or crossbred to produce colonies of the particular animal. Examples of such breeding strategies include but are not limited to: outbreeding of founder animals with more than one integration site in order to establish separate lines; inbreeding of separate lines in order to produce compound PD-related gene transgenics that express the PD-related gene transgene of interest at higher levels because of the effects of additive expression of each PD-related gene transgene; crossing of heterozygous transgenic animals to produce animals homozygous for a given integration site in order both to augment expression and eliminate the possible need for screening of animals by DNA analysis; crossing of separate homozygous lines to produce compound heterozygous or homozygous lines; breeding animals to different inbred genetic backgrounds so as to examine effects of modifying alleles on expression of the PD-related gene transgene and the development of neurodegenerative disease symptoms. One such approach is to cross the PD-related gene transgenic founder animals with a wild type strain to produce an F1 generation that exhibits PD-related disease symptoms. The F1 generation may then be inbred in order to develop a homozygous line, if it is found that homozygous PD-related gene transgenic animals are viable.

Any of the animal models disclosed herein can be used to identify agents capable of ameliorating, treating or preventing symptoms associated with susceptibility to PD-related disease. For example, animal models can be exposed to a compound suspected of exhibiting an ability to ameliorate one or more symptoms associated with PD-related disease (e.g., tremor of the hands, arms, legs, jaw and face (resting tremor); muscular rigidity; slowness of movement (bradykinesia); impaired balance and coordination (postural instability); dysautonomia, dystonic cramps; dementia; a loss of dopaminergic neurons; and the presence of intracellular inclusions in surviving neurons in various areas of the brain, etc.) at a sufficient concentration and for a time sufficient to elicit an ameliorating response in the exposed animal. The response of the exposed animal can be monitored by assessing change in symptoms. Any treatments that diminish one or more symptoms associated with PD-related disease or susceptibility thereto may be considered a candidate for human therapy. Dosages of test agents can be determined by deriving dose-response curves, which are well known and commonly used in the art.

Cell-based systems can also be useful for identifying agents that ameliorate symptoms associated with PD-related disease. Cell-based systems include cells that express one or more of the PD-related disease polypeptides herein and exhibit cellular phenotypes associated with resistance or susceptibility to PD-related disease. Cell-based systems include recombinant transgenic cell lines derived from animals containing one or more cells expressing one or more of the nucleic acids herein. For example, the PD-related disease animal models of the invention, discussed above, may be used to generate cell lines, containing one or more cell types involved in PD-related disease that can be used as cell culture models for this disease. While primary cultures derived from the PD-related disease transgenic animals of the invention may be utilized, the generation of continuous cell lines is preferred. For examples of techniques which may be used to derive a continuous cell line from the transgenic animals, see Small et al. (1985) Mol. Cell Biol. 5:642-648. Cell-based systems also include non-recombinant cell lines preferably from primary tissues of patients having PD-related disease or resistance to PD-related disease. Cell lines suitable for development of a cell-based system include, but are not limited to U937 (ATCC# CRL-1593), THP-1 (ATCC# TIB-202), and P388D1 (ATCC# TIB-63); endothelial cells such as HUVEC's and bovine aortic endothelial cells (BAEC's); as well as generic mammalian cell lines such as HeLa cells and COS cells, e.g., COS-7 (ATCC# CRL-1651).

Alternatively, cells of a cell type known to be involved in PD-related disease may be transfected with sequences capable of increasing or decreasing the amount of PD-related disease gene expression within the cell. For example, PD-related disease gene sequences may be introduced into, and overexpressed in, the genome of the cell of interest, or, if endogenous PD-related disease gene sequences are present, they may be either overexpressed or, alternatively disrupted in order to underexpress or inactivate PD-related disease gene expression.

In order to overexpress a PD-related disease gene sequence, the coding portion of the PD-related disease gene sequence may be ligated to a regulatory sequence which is capable of driving gene expression in the cell type of interest. Such regulatory regions will be well known to those of skill in the art, and may be utilized in the absence of undue experimentation. For underexpression of an endogenous PD-related disease gene sequence, such a sequence may be isolated and engineered such that when reintroduced into the genome of the cell type of interest, the endogenous PD-related disease gene alleles will be inactivated. Preferably, the engineered PD-related disease gene sequence is introduced via gene targeting such that the endogenous PD-related disease sequence is disrupted upon integration of the engineered PD-related disease gene sequence into the cell's genome.

Cells treated with compounds or transfected with PD-related disease genes can be examined for phenotypes associated with PD-related disease. Similarly, HUVEC's may be treated with test agents or transfected with genetically engineered PD-related disease genes. The HUVEC's can then be examined for phenotypes associated with PD-related disease, including, but not limited to changes dopamine production, apoptosis, etc.; or for the effects on production of other proteins involved in PD-related disease such as α-synuclein, parkin, ubiquitin C-terminal hydrolase, DJ-1, etc.

Transfection of PD-related disease gene sequence nucleic acids may be accomplished by utilizing standard techniques. See, for example, Ausubel (1989) supra. Transfected cells should be evaluated for the presence of the recombinant PD-related disease gene sequences, for expression and accumulation of PD-related disease gene mRNA, and for the presence of recombinant PD-related disease gene protein production. In instances wherein a decrease in PD-related disease gene expression is desired, standard techniques may be used to demonstrate whether a decrease in endogenous PD-related disease gene expression and/or in PD-related disease gene product production is achieved.

A cell-based system having a phenotype of PD-related disease can be exposed to an agent suspected of ameliorating phenotypic states associated with susceptibility to PD-related disease at a sufficient concentration and for a time sufficient to elicit such an amelioration response in the exposed cells. After exposure, the cells can be examined to determine whether the phenotypic states have been altered such that the phenotype has been eliminated and the cells resemble normal phenotypes or phenotypes of resistance to PD-related disease.

V. Pharmaceutical Compositions

Any of the agents and compositions identified herein may be produced in quantities sufficient for pharmaceutical administration and/or testing.

Pharmaceutical compositions can be formulated in accordance with the routine procedures adapted for administration to human beings. Often, pharmaceutical compositions are formulated with an acceptable carrier or excipient. See Remington's Pharmaceutical Sciences, Gennaro, A., (ed., Mack Publishing Co. 1990).

Suitable pharmaceutically acceptable carriers include but are not limited to water, salt solutions (e.g., NaCl),. saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof.

Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The pharmaceutical compositions can include, if desired, auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The pharmaceutical compositions, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides.

The pharmaceutical compositions and their physiologically acceptable salts and solvates can be formulated for administration by inhalation or insufflation (either through the mouth or the nose, or oral, buccal, parenteral, or rectal administration). For administration by inhalation, the compositions are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

For oral administration, the pharmaceutical compositions can take the form of tablets or capsules prepared by conventional means with pharmaceutically acceptable excipients such as binding agents, fillers, disintegrants, or wetting agents, sweeteners, including, pregelatinised maize starch, polyvinylpyrrolidone, hydroxypropyl methylcellulose, fillers, lactose, microcrystalline cellulose, calcium hydrogen phosphate, lubricants, magnesium stearate, talc, silica, potato starch or sodium starch glycolate, sodium lauryl sulfate, mannitol, lactose, starch, magnesium stearate, polyvinyl pyrollidone, sodium saccharine, cellulose and magnesium carbonate. The tablets can be coated by methods well known in the art. Preparations for oral administration can be suitably formulated to give controlled release of the active compound.

Liquid preparations for oral administration can take the form of solutions, syrups, or suspensions, or they can be presented as a dry product for constitution with water or other suitable vehicle before use. Such liquid preparations can be prepared by conventional means with pharmaceutically acceptable additives such as suspending agents, e.g., sorbitol syrup, cellulose derivatives, or hydrogenated edible fats; emulsifying agents, e.g., lecithin or acacia; non-aqueous vehicles, e.g., almond oil, oily esters, ethyl alcohol, or fractionated vegetable oils; and preservatives, e.g., methyl or propyl-p-hydroxybenzoates or sorbic acid. The preparations can also contain buffer salts, flavoring, coloring, and/or sweetening agents as appropriate.

In particular, the liquid preparations can be administered in a beverage. Such beverage can be alcoholic, non-alcoholic beverage or a health beverage. Such beverage may comprise one or more of the agents or compositions herein as well as, optionally, any one or more of the following: alcohol fructose, vitamins, electrolyte substitutes, caffeine, amino acids, minerals, artificial and natural sweeteners, milk or dry-milk powder and other additives and preserving agents.

Examples of vitamins that may be included are components of the vitamin B complex, such as vitamin B1, B2, B6, B12, biotin, niacin, pantothenic acid, folic acid, adenine, choline, adenosine phosphate, orotic acid, pangamic acid, carnitine, 4-aminobenzoic acid, myo-inositol, liponic acid and/or amygdaline. In the body, vitamin B1, also known as thiamin, is converted into thiamin-pyrophosphate, a coenzyme in a number of reactions in which C—C bonds are cleaved. It can also be added as thiamin hydrochloride. Vitamin B2, also known as riboflavin, is reabsorbed in the small intestines, converted into FMN (flavin mononucleotide) and, in the liver, into FAD (flavin-adenine-dinucleotide), both of which are coenzymes in redox reactions. Vitamin B6, also known as pyridoxal, pyrodoxin and pyridoxamine, is a component of pyridoxal-5-phosphate, which is a cofactor in glycogen degradation and in amino acid metabolism, e.g. as a coenzyme of decarboxylases. Preferably, this substance is admixed into the beverage in the form of pyridoxin hydrochloride. Vitamin B12, also known as cyanocobalamine, has a complex structure and is a component of cobalamine-coenzymes, with methyl-cobalamine and cobalamide, e.g., being involved in rearrangements with hydrogen migration. Biotin, also known as vitamin B7, is covalently bound to carboxylases. Niacin, also known as B3, is a generic name for nicotinic acid and nicotinamide. Niacin is a component of NAD and its phosphate, NADP, and is one of the most important hydrogen transmitters in the cell having a protective and anabolic effect on the body. Pantothenic acid, also known vitamin B3 or B5, has a precursor function for coenzyme A which assumes a central position in metabolism. Folic acid, or vitamin B9, is a component of the coenzyme tetrahydrofolate. Vitamin C may further be provided.

Preferably, the beverage composition comprises components of the vitamin B complex in the following parts by weight, based on a total of 15,000-20,000 parts by weight of the dry substance: vitamin B 1, 0.1 - 10 parts by weight, preferably 1 part by weight; vitamin B2, 0.1-10 parts by weight, preferably 1.5 parts by weight; vitamin B6, 0.1-10 parts by weight, preferably 1.5 parts by weight; biotin, 0.01-1 parts by weight, preferably 0.1 parts by weight; niacin, 0.1-100 parts by weight, preferably 10-30 parts by weight; pantothenic acid, 0.1-100 parts by weight, preferably 1-10 parts by weight; vitamin B12, 0.0001-0.1 parts by weight, preferably 0.001-0.01 parts by weight; folic acid, 0.01-10 parts by weight, preferably 0.1 parts by weight, and/or vitamin C, 0.1-500 parts by weight, preferably 50 parts by weight.

It is advantageous for the beverage to comprise of amino acids, in particular L-glutamine and/or L-arginine. Amino acids play an important role in the various metabolic processes of the human body. In particular, L-glutamine and L-arginine may be admixed in the beverage according to the following parts by weight, based on a total of 15,000-20,000 parts by weight of dry substance: L-arginine, 20-2,000 parts by weight, preferably 200 parts by weight; and/or L-glutamine, 10-1,000 parts by weight, preferably 100 parts by weight.

Caffeine is optionally added at 0.1-100 parts by weight, preferably 25 parts by weight, based on a total of 15,000-20,000 parts by weight.

Examples of mineral that may be used include magnesium, potassium, zinc and calcium. In particular, potassium and magnesium play an important role in metabolism and are involved in many ATP-catalyzed enzyme reactions. Minerals may be added separately, in combination, and/or in combination with other food additives, e.g. as magnesium glycerophosphate, potassium citrate (acid regulator), zinc gluconate (fruit acid) and calcium pantothenate. Minerals are preferably added at the following parts by weights, based on a total of 15,000-20,000 parts by weight of the dry substance: magnesium, 10-1,000 parts by weight, preferably 100 parts by weight; potassium 10-1,000 parts by weight, preferably 100 parts by weight; zinc, 0.1-100 parts by weight, preferably 10 parts by weight; calcium 10-1,000 parts by weight, preferably 100 parts by weight.

A tastier beverage may further include sugars and/or artificial sweeteners. Both artificial and natural sweeteners may be added to sweeten the compositions herein. Besides fructose, any other sugar may be admixed, such as glucose, galactose, lactose, etc. Artificial sweeteners include, for example, aspartame, saccharine and cyclamate as well as any other commercially available artificial sweeteners.

Furthermore, the compositions herein may comprise of further additives, in particular flavoring agents, preserving agents, coloring agents, antioxidants, electrolytes, enzymes, plant extracts, glycerolphosphates, acid regulators and/or acidifiers, in particular fruit acids.

A beverage may be carbonated or non-carbonated, and may be combined or based on liquids such as fruit juices, milk, tea, coffee, water etc. Moreover, alcohol may be admixed to the beverage herein.

The compositions can be formulated for intravenous administration. Compositions used for intravenous administration are typically solutions in sterile isotonic aqueous buffer. Where necessary, the compositions may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule indicating the quantity of active agent. Where the composition is to be administered by infusion, it can be dispensed with an infusion bottle containing sterile pharmaceutical grade water, saline or dextrose/water. Where the compositions are administered by injection, an ampule of sterile water for injection or saline can be provided so that the ingredients may be mixed prior to administration.

The compositions can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions, or emulsions in oily or aqueous vehicles, and can contain formulatory agents such as suspending, stabilizing, and/or dispersing agents. Alternatively, the active ingredient can be in powder form for constitution with a suitable vehicle, e.g., sterile pyrogen-free water, before use.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carver material, is packaged in a squeeze bottle or in admixture with a pressurized volatile, normally gaseous propellant, e.g., pressurized air.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration. Pharmaceutical packs or kits comprising one or more containers filled with one or more of the ingredients of the pharmaceutical compositions disclosed herein are also provided. Optionally, associated with such containers can be a notice in the form prescribed by a governmental agency regulating the manufacture, use or sale of pharmaceuticals or biological products, which notice reflects approval by the agency of manufacture, use of sale for human administration. The packs or kits can be labeled with information regarding mode of administration, sequence of drug administration (e.g., separately, sequentially or concurrently) or the like. The packs or kits may also include means for reminding the patient to take the therapy. The packs or kits can comprise of a single unit dosage of the-combination therapy or a plurality of unit dosages. In particular, the compositions can be separated, mixed together or present in a single vial or tablet. Compositions assembled in a blister pack or other dispensing means are preferred. Unit dosages provided are preferably dependent on the pharmacodynamics of each agent and administered in FDA approved dosages in standard time courses.

VII. Methods for Treatment

The agents and pharmaceutical compositions herein can be used as prophylactic or therapeutic treatment of PD-related disease. PD-related disease may result from excessive levels of certain gene products (e.g., polypeptides associated with susceptibility to PD-related disease) or deficient levels of other gene products (e.g., polypeptides associated with resistance to PD-related disease).

1. Indications for Treatment

Preferable indications for treatment involve e.g. by tremor of the hands, arms, legs, jaw and face (resting tremor); muscular rigidity; slowness of movement (bradykinesia); impaired balance and coordination (postural instability); dysautonomia, dystonic cramps; dementia; a loss of dopaminergic neurons; and the presence of intracellular inclusions in surviving neurons in various areas of the brain, especially those associated with PD-related disease.

2. Methods for Administration

The agents and pharmaceutical compositions herein can be administered separately or in combination, in an amount effective to treat an indication of interest. For example, a patient diagnosed with or afflicted by a PD-related disease, may be administered a therapeutically effective amount of an inhibitor of polypeptides associated with susceptibility to PD-related disease to reduce the level of activity and/or expression of such polypeptides. In the alternative, a patient diagnosed with or afflicted by a PD-related disease, may be administered a therapeutically effective amount of an agonist of polypeptides associated with resistance to PD-related disease to increase the level of activity and/or expression of such polypeptides. More preferably, a patient diagnosed with or afflicted by a PD-related disease, is administered a combination treatment of both inhibitors of polypeptides associated with susceptibility to PD-related disease and agonists of polypeptides associated with resistance to PD-related disease. Such combination treatment may require lower dosages due to the synergetic effect of both compounds.

The agents and pharmaceutical compositions may be administered or co-administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions may also be administered or co-administered in slow release dosage forms. Other suitable methods include gene therapy using rechargeable or biodegradable devices, particle acceleration devices (“gene guns”) and slow release polymeric devices. The pharmaceutical compositions herein can also be administered as part of a combinatorial therapy with other agents.

The combination of therapeutic agents and compositions may be administered by a variety of routes, and may be administered or co-administered in any conventional dosage form. Co-administration in the context of this invention is defined to mean the administration of more than one therapeutic in the course of a coordinated treatment to achieve an improved clinical outcome. Such co-administration may also be coextensive, that is, occurring during overlapping periods of time. For example, a associated genomic region antisense may be administered to a patient before, concomitantly, or after the administration of an inhibitor of PD-related disease polypeptides.

In a preferred embodiment, a pharmaceutical compound is administered orally, and more preferably is self-administered. For example, a beverage comprising one or more agents or pharmaceutical compositions may be administered to prevent, ameliorate or treat PD-related disease. The dosage of active ingredients may be based on the composition, its interaction with other compounds, or more preferably the blood pressure of a patient.

3. Dosage

The amounts of therapeutic agents or compositions to be administered can vary, according to determinations made by one of skill, but preferably are in amounts effective to reduce or reverse progression of PD-related disease. Treatment compositions and dosages can be specifically tailored to each situation based on an individual patient's pharmacogenomics (response to a drug), phenotype, genotype and the compositions used for treatment. Preferably, for co-administration, the total amounts are less than the total amounts for each pharmaceutical compound added together. For the slow-release dosage form, appropriate release times can vary, but preferably should last from about 1 hour to about 6 months, most preferably from about 1 week to about 4 weeks. Formulations for slow release dosage can vary as determinable by one of skill, according to the particular situation and as generally taught herein.

The LD₅₀ (the lethal dose to 50% of the population) and the ED₅₀ (the effective dose in 50% of the population) of a pharmaceutical composition can be determined using cell cultures or animal models following standard pharmaceutical procedures. The dose ratio of lethal and effective doses is the therapeutic index and is expressed as the ratio LD₅₀/ED₅₀ Compounds that exhibit large therapeutic indices are preferred. Compounds that exhibit toxic side effects can also be used, but care should be taken to design a delivery system that targets such compounds to the site of affected tissue to minimize potential damage to unaffected cells and tissues.

When using cell culture to estimate the therapeutically effective dose, the dosage of such compounds lies preferably within a range of circulating concentrations that include the ED₅₀ with little or no toxicity. A dose can also be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography.

4. Gene Replacement Therapy

In another embodiment, nucleic acids can be introduced into recipient cells using techniques such as gene replacement therapy.

Preferably, one or more nucleic acids associated with resistance to PD-related disease may be inserted into appropriate cells within a patient, using vectors such as adenovirus, adeno-associated virus and retrovirus vectors. Nucleic acids can also be introduced into cells via particles, such, as liposomes. Other techniques for direct administration involve stereotactic delivery of such sequences to the site of the cells in which the sequences are to be expressed.

Methods for introducing nucleic acids into mammalian cells are well known in the art. Generally, the nucleic acid is directly administered in vivo into a target cell or a transgenic mouse that expresses SP-10 promoter operably linked to a reporter gene. This can be accomplished by any methods known in the art, e.g., by constructing it as part of an appropriate nucleic acid expression vector and administering it so that it becomes intracellular, e.g., by infection using a defective or attenuated retroviral or other viral vector (U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus, or by administering it in linkage to a ligand subject to receptor-mediated endocytosis (Wu and Wu, (1987) J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; W092/20316 dated Nov. 26, 1992; W093/14188 dated Jul. 22, 1993; WO 93/20221 dated Oct. .14, 1993).

Additional methods that may be utilized to increase or decrease the overall level of expression of a nucleic acid include using targeted homologous recombination methods to modify the expression characteristics of an endogenous sequence in a cell or microorganism by inserting a heterologous DNA regulatory element such that the inserted regulatory element is operatively linked with the endogenous sequence in question. Targeted homologous recombination can thus be used to activate transcription of an endogenous nucleic acid that is transcriptionally silent, (e.g., not normally expressed or expressed at very low levels), to silence the transcription of an endogenous nucleic acid that is transcriptionally active, or to enhance or decrease the expression of an endogenous sequence that is normally expressed.

Further, the overall level of expression of polypeptides associated with resistance to PD-related disease may be increased by the introduction of cells that express such polypeptides associated with resistance to PD-related disease, preferably autologous cells, into a patient at positions and in numbers which are sufficient to prevent or ameliorate symptoms or conditions associated with PD-related disease. Such cells may be either recombinant or non-recombinant. In a preferred embodiment, such cells are healthy neural cells.

When the cells to be administered are non-autologous cells, they can be administered using well-known techniques that prevent a host immune response against the introduced cells from developing. For example, the cells may be introduced in an encapsulated form that, while allowing for an exchange of components with the immediate extracellular environment, does not allow the introduced cells to be recognized by the host immune system.

4. Kits

The combination of therapeutic agents may be used in the form of kits. The arrangement and construction of such kits is conventionally known to one of skill in the art. Such kits may include containers for containing the inventive combination of therapeutic agents and/or compositions, and/or other apparatus and instructions for administering the inventive combination of therapeutic agents and/or compositions.

VIII. Prognostic, Diagnostic and Theranostic Uses

Preventative measures may be successful in preventing some PD-related diseases, but these measures are only successful if individuals can be identified as at risk of developing the disease before onset of the disease. The onset of PD-related diseases is especially difficult to predict due to the complex set of factors that influence their development. As such, individuals often do not know they are at risk of developing a PD-related disease until it is too late to prevent it. It will be clear to one of skill in the art that the PD-related disease polymorphisms presented may serve as valuable tools for clinicians in making medical decisions regarding the care of their patients. Theranostics is a combination of the separate disciplines of diagnostics and therapeutics, and describes the use of diagnostics to guide therapy decisions in treating patients.

In certain embodiments, the present invention provides methods for identifying individuals at risk of developing a PD-related disease (prognostics), thereby allowing implementation of measures to prevent or delay the onset of the disease. In one embodiment, an individual's risk of developing a PD-related disease may be determined genotyping the individual for the PD-related disease polymorphisms provided herein or a subset thereof. If an individual possesses one or more alleles that are associated with PD-related disease, the institution of preventative measures (e.g., drug therapies) may be justified. This information may be used by a clinician to better determine an appropriate treatment regimen for the individual. Often, this information is used in combination with clinical information regarding the disease, the patient, or the population from which the patient comes. In some aspects, the PD-related disease polymorphisms presented herein may also be used to identify individuals who are resistant to a PD-related disease. For example, some individuals who have a family history of a PD-related disease (e.g., PD, Alzheimer's disease, etc.) never develop the disease. An individual may be genotyped for the PD-related disease polymorphisms provided herein, and if the individual does not possess one or more alleles that are associated with PD-related disease, then institution of preventative measures may not be justified. As will be clear to one of skill in the art, this knowledge would allow clinicians to better assess the risk of individuals of developing the PD-related disease in question, provide peace of mind to those who are not at high risk, and in some cases would preclude unnecessary prophylactic treatments (e.g., drug therapy). The PD-related disease polymorphisms presented herein may also be used to identify individuals with an increased risk of developing a PD-related disease and thereby motivate life-style changes to prevent onset of the disease. For example, a polygenic test comprising set of PD-related disease polymorphisms associated with PD could provide strong incentive to those who are found to be at high risk to avoid chemicals or drugs associated with the onset of PD-related disease (e.g. methanol, carbon monoxide, cyanide, carbon disulfide, manganese, meperidine, lithium, dopamine receptor blockers, dopamine depleters, etc.)

In other embodiments, the PD-related disease polymorphisms presented herein may be used to aid in the determination of whether or not a prophylactic therapy is warranted to prevent development of e.g. a disease in an individual. For example, there are approved therapeutics for prevention of certain neurological disorders that are dependent on historical clinical information such as family history, clinical testing, etc. These factors, although useful for computing a pre-test odds, are only marginally predictive of whether or not a person will develop a neurodegenerative disorder. A genetic test to be used in combination with the pre-test odds would provide a far superior means of deciding whether or not to treat an individual prophylactically (e.g. with Levodopa, dopamine agonists, COMT inhibitors, anticholinergics, MAO-B inhibitors, antiviral agents, vitamin E, hormone replacement therapy, etc.) by providing a much more accurate way to identify and quantify their risk of developing a neurodegenerative disorder.

In one aspect of the present invention, a prognostic or diagnostic assay is provided comprising a nucleic acid array that contains probes designed to detect the presence of the set of PD-related disease polymorphisms in a biological sample. Nucleic acids are isolated from a biological sample from a test individual and are hybridized to the probes on the nucleic acid array. The probe intensities are analyzed to provide a genotype for the test individual at each of the PD-related disease polymorphisms. The genotypes are used to determine the individual's risk of developing the disease by methods known in the art or those described in U.S. patent application Ser. No. 60/566,302, filed Apr. 28, 2004; Ser. No. 60/590,534, filed Jul. 22, 2004; and Ser. No. 10/956,224, filed Sep. 30, 2004, all of which are entitled “Methods for Genetic Analysis”.

For example, a polymorphic profile may be determined and analyzed to determine an individual's genetic risk of developing a PD-related disease. A polymorphic profile refers to the matrix of variant forms occupying one or more polymorphic sites. The profile can be determined on at least 1, 2, 5, 10, 25, 35, 50, 100, or all of polymorphic sites shown in Table 1, and optionally others in linkage disequilibrium with them. The polymorphic profile is preferably determined in at least 1, 2, 5, 10, 25 or all of the polymorphic sites shown in Table 1. For polymorphic sites in linkage disequilibrium with a polymorphic site shown in Table 1, the polymorphic site preferably occurs in the same gene as shown in Table 1 or proximal thereto. The polymorphic profile preferably includes polymorphic sites from at least 2, 5, 10, 15, 25 or all of the genes shown in Table 1. The polymorphic sites of the invention can be analyzed in combination with other polymorphic sites. However, the total number of polymorphic sites analyzed is usually less than 10,000, 1000, 100, 50 or 25.

The number of alleles associated positively or negatively with a given phenotype present in a particular individual can be combined additively or as ratio to provide an overall score for the individual's genetic propensity to the phenotype (see U.S. Ser. No. 60,566,302, filed Apr. 28, 2004, U.S. Ser. No. 60/590,534, filed Jul. 22, 2004, U.S. Ser. No. 10/956,224 filed Sep. 30, 2004, and PCT US 05/07375 filed Mar. 3, 2005). For example, alleles associated with a susceptibility to PD-related disease can be arbitrarily each scored as +1 and alleles associated with a resistance to PD-related disease as −1 (or vice versa). For example, if an individual is typed at 30 polymorphic sites of the invention and is homozygous for alleles associated with a susceptibility to PD-related disease at all of them, he could be assigned a score of 100% genetic risk of developing PD-related disease. The reverse applies if the individual is homozygous for all alleles associated with a resistance to PD-related disease. More typically, an individual is homozygous for susceptibility alleles at some loci, homozygous for resistance alleles at some loci, and heterozygous for susceptibility and resistance alleles at other loci. Such an individual's genetic risk of developing PD-related disease can be scored by assigning all susceptibility alleles a score of +1, and all resistance alleles a score of −1 (or vice versa) and combining the scores. For example, if an individual has 40 susceptibility alleles and 20 resistance alleles, the individual can be scored as having a 67% genetic risk of developing PD-related disease. Alternatively, homozygous susceptibility alleles can be assigned a score of +1, heterozygous alleles a score of zero and homozygous resistance alleles a score of −1. The relative numbers of resistance alleles and susceptibility alleles can also be expressed as a percentage. Thus, an individual who is homozygous for susceptibility alleles at 20 polymorphic sites, homozygous for resistance alleles at 40 polymorphic sites, and heterozygous at 10 sites is assigned a genetic risk of 33% for developing PD-related disease. As a further alternative, homozygosity for susceptibility alleles can be scored as +2, heterozygosity, as +1 and homozygosity for resistance alleles as 0.

In other embodiments, the set of PD-related disease polymorphisms identified herein are used for pharmacogenomics and drug development. Due to the great number of treatment options available for PD-related diseases, it is often difficult to determine which of a group of treatment options will be most effective for a given patient. Typically, several different options must be tried before one or a combination of two or more is found that is safe and effective. In the meantime, the patient will continue to suffer the effects of the disease, and perhaps will also experience adverse events in response to one or more of the treatment options tested. The PD-related disease polymorphisms presented herein are useful for stratifying patient populations prior to initiation of a treatment regimen. PD-related disease polymorphisms are identified that are associated with the response of a patient to a drug or other medical treatment. The response may be, e.g., an adverse event or may be related to the efficacy of the treatment. The PD-related disease polymorphisms are used to screen patient populations to generate genetic profiles for the patients that will help clinicians determine which individuals should be given the drug or medical treatment and which should not. For example, individuals who are predisposed to exhibiting an adverse event and individuals who are unlikely to have an efficacious response to a drug may be excluded from treatment with a given drug, and may instead be treated by alternate means (different drug or other medical treatment).

In one such embodiment, individuals are screened for a set of PD-related disease polymorphisms that are associated with a disease that confers a known risk of an adverse response to a particular drug treatment. Those individuals at high risk of developing the disease are excluded from the treatment regimen. For example, administering lithium, dopamine receptor blockers, or dopamine depleters to an individual who is genetically predisposed to developing a PD-related disease may increase their risk or accelerate the development of the disease. As such, it would be beneficial to screen a patient population for a set of loci associated with PD-related disease prior to administering such a drug, and to either exclude those individuals at high risk of developing PD-related disease or monitor them closely for development of such a disease. Thus, the presence of PD-related disease polymorphisms that predispose an individual to a disease that is characterized by a high risk of an adverse event is information that may be used by a clinician to determine appropriate treatment options for the individual. For example, if the individual has a high risk of developing PD, then administration of dopamine receptor blockers may be avoided. If the individual has a low risk of developing PD, then administration of dopamine receptor blockers may be a viable treatment option.

In another embodiment of the present invention, the effectiveness of a drug treatment regimen is predicted for an individual based on the genotypes of the individual at a set of PD-related disease polymorphisms that are associated with efficacy of the drug. This information is used to determine a probability of whether the drug will be an effective treatment for the individual, or if other drugs or treatment options should be considered instead. For example, an association study may be performed using a case group of individuals that do not have an efficacious response to the drug (“nonresponders”) and a control group of individuals that have an efficacious response (“responders”). Members of the case and control groups are genotyped at a plurality of PD-related disease polymorphisms, relative allele frequencies are computed for each of the polymorphisms, and a set of polymorphisms associated with an efficacious response is identified as those polymorphisms that have allele frequency differences that are significantly different between the case and control groups. An individual who is a candidate for receiving the drug is genotyped at each of the PD-related disease polymorphisms that are associated with the efficacy of the drug, and the likelihood that the individual will have an efficacious response to the drug is determined based on his/her genotypes at the set of associated SNPs. This information can then be used by a clinician in deciding on appropriate treatments for the individual.

In a related embodiment, a diagnostic may be developed for a therapeutic area (e.g., one or more PD-related diseases) to enable a clinician to better individualize treatment of patients. Rather than focusing on a single drug, the therapeutic area diagnostic would provide information on the likelihood that a patient will be a responder for a series of drugs related to a single therapeutic area. For example, there are a multitude of drugs on the market for treating PD-related disease including Levodopa, dopamine agonists, COMT inhibitors, anticholinergics, MAO-B inhibitors, antiviral agents, vitamin E and hormone replacement therapies. Association studies may be performed to identify PD-related disease polymorphisms associated with the efficacy of each of these types of drugs, and those polymorphisms could then be used to screen patient populations to determine which class of drugs would be most efficacious for a given individual. For each drug class, a case group comprises individuals with a given. PD-related disease that had an efficacious response to the drug class, and a control group comprises individuals that did not have an efficacious response to the drug class. PD-related disease polymorphisms that are associated with drug efficacy are identified as those that have a significantly different allele frequency in the cases than in the controls. An individual in need of PD-related disease therapy is screened for the polymorphisms that are associated with efficacy for each of the drug classes, and a clinician determines an appropriate therapy choice for the individual based on the individual's genotype information, as well as other patient-specific information.

In yet another embodiment, the methods presented herein may be used to assess whether a brand name drug should be used, or if a cheaper generic may be substituted instead. For example, an association study would be performed to identify PD-related disease polymorphisms associated with a positive clinical response to the generic alternative. A patient in need of treatment would be genotyped at these associated loci, the genotyping results would be used to predict the efficacy of the generic drug in the individual, and a clinician would use this information to make a treatment decision for the individual. This application of the disclosed methods could be used for medical costs reimbursement decisions, as well. For example, if it was found that the generic drug was unlikely to be efficacious in individual A, then the brand name drug would be administered to A and the cost of the brand name drug could be reimbursed to A; however, if individual B was likely to have an efficacious response to the generic, then individual B would not be given the more expensive brand name drug, and only the cost of the generic would be reimbursable.

In another embodiment of the present invention, the likelihood that an individual will experience an adverse event in response to administration of a drug is determined based on the genotypes of the individual at a set of PD-related disease polymorphisms associated with the occurrence of adverse events related to the drug. If an individual is found to have a high risk of experiencing an adverse event in response to a treatment regimen, then the treatment regimen may be avoided and other treatment options may be considered. For example, an association study may be performed using a case group of individuals that exhibited an adverse event in response to the drug and a control group of individuals that did not exhibit the adverse event. Members of the case and control groups are genotyped at a plurality of PD-related disease polymorphisms, relative allele frequencies are computed for each of the polymorphisms, and polymorphisms associated with the adverse event are identified as those that have allele frequency differences that are significantly different between the case and control groups. Prior to receiving the drug, an individual is genotyped at each of the PD-related disease polymorphisms that are associated with the adverse event, and a clinician determines an appropriate treatment regimen based, at least in part, on the genotyping results. For example, if the individual is found to have a high risk, the use of the drug may be avoided. In some aspects, the individual may be classified as having an intermediate likelihood of experiencing an adverse event and alternative drug therapies may be used, or the drug may be administered e.g. only with close monitoring, or in combination with another therapeutic to counteract the adverse event. Determination of the best treatment regimen for an individual with an intermediate risk of experiencing an adverse event may rely more heavily on other information (e.g. clinical data, FDA or patient input, etc.) than does determination of the best treatment regimen for an individual with a very high or low risk. This information can then be used by a clinician in deciding on appropriate treatments for the individual. Adverse events in response to administration of a drug include, but are not limited to, allergic reactions, cardiac arrhythmia, stroke, bronchospasm, gastrointestinal disturbances, fainting, impotency, rashes, fever, muscle pain, headaches, nausea, birth defects, hot flashes, mood changes, dizziness, agitation, vomiting, sleep disturbance, somnolence, insomnia, addiction to the drug, and death.

In a related embodiment, PD-related disease polymorphisms associated with the safety of a drug may be used to improve the safety of the drug by stratifying patient populations to exclude from treatment those individuals likely to exhibit an adverse event in response to administration of the drug. In one example, a new drug is found to have excellent efficacy, tolerance and convenience, however 4% of individuals treated with the drug experience a severe adverse event, and this incidence of adverse events has limited the use of the drug, e.g. to only those individuals for whom other therapies have failed. However, a regulatory agency has stipulated that if the incidence of the adverse event were lowered by at least 50% then the drug could be approved for wider usage. This could be achieved if individuals who are likely to experience the adverse event could be identified prior to treatment, so an association study is performed with a case group of individuals that experienced the adverse event and a control group of individuals that did not to identify a set of PD-related disease polymorphisms associated with the adverse event. This set of polymorphisms is then used to screen patients in order to identify and exclude those likely to exhibit an adverse event from a patient population, thereby reducing the number of adverse events in the treated population and effectively increasing the safety of the drug.

IX. Further Study of PD-Related Disease

The set of PD-related disease polymorphisms may further be used for identifying regions of the genome that are involved in development of a PD-related disease phenotype. These associated polymorphisms (e.g., associated SNPs) may be directly involved in the manifestation of a PD-related disease, or they may be in linkage disequilibrium with other loci that are directly involved. For example, a PD-related disease polymorphism may affect the expression or function of a PD-related disease protein directly, or may be in linkage disequilibrium with another locus that affects the expression or function of the protein. Examples of direct effects to the expression or function of a protein include, but are not limited to, a polymorphism that alters the polypeptide sequence of the protein, and a polymorphism that occurs in a regulatory region (i.e., promoter, enhancer, etc.) resulting in the increased or decreased expression of the protein. In certain embodiments, genomic regions containing the set of associated SNPs are analyzed to identify genes that are directly involved in the biological basis of the disease.

The PD-related disease polymorphisms that lie in the coding region of a gene may be used to detect or quantify expression of a PD-related disease allele in a biological specimen for use as a diagnostic marker for the disease. For example, nucleic acids containing the PD-related disease polymorphisms may be used as oligonucleotide probes to monitor RNA or mRNA levels within the organism to be tested or a part thereof, such as a specific tissue or organ, so as to determine if the gene encoding the RNA or mRNA contains an allele associated with PD-related disease. In one aspect, a diagnostic or prognostic kit is provided that comprises oligonucleotide probes for use in detecting an PD-related disease allele in a biological sample. Likewise, if the PD-related disease allele causes a change in the polypeptide sequence of the encoded protein, the allelic constitution of the gene may be assayed at the protein level using any customary technique such as immunological methods (e.g., Western blots, radioimmune precipitation and the like) or activity-based assays measuring an activity associated with the gene product. In one aspect, a diagnostic or prognostic kit is provided that comprises an assay for detecting a polypeptide encoded by a PD-related disease allele in a biological sample. The manner in which cells are probed for the presence of particular nucleotide or polypeptide sequences is well established in the literature and does not require further elaboration here, however, see, e.g., Sambrook, et al., Molecular Cloning: A Laboratory Manual (Cold Spring Harbor Laboratory, New York) (2001).

X. An Exemplary Study Design

In one exemplary study design, susceptibility genes for Parkinson's disease (PD) are identified by performing a genome-wide association study. PD is a complex disease arising from genetic (˜45% heritable) and environmental interactions. Approximately 200,000 (e.g., 198,352) SNPs are genotyped in ˜500 (e.g., 443) sibpairs discordant for PD (affected individuals and their unaffected siblings). The 200,000 SNP markers used in this initial screen are selected because they are uniformly distributed across the genome and because they have allele frequencies greater than 10% in multiple populations. The ˜2000 (e.g., 1525) markers having the most significant allele frequency differences (e.g., p <0.01) between PD affected individuals and their unaffected siblings are genotyped in an additional population consisting of approximately 300 (e.g., 332) individuals with PD and 300 well-matched, unrelated controls. In addition to the ˜2000 markers, an additional set of ˜300 (e.g., 311) genomic control SNPs were also genotyped to ensure that the populations were adequately matched (see, e.g., PCT patent application no. US04/13577, filed Apr. 30, 2004). This two-tiered association study design provides sufficient power (˜80%) to identify all the major PD susceptibility genes and to identify risk factor profiles associated with a high risk for PD. The discovery of novel susceptibility genes defines pathogenesis pathways and provides new targets for disease modifying therapies, the end result being pragmatic primary and secondary prevention strategies for PD.

Identification of Polymorphisms

The entire human genome may be scanned to identify common polymorphisms (and others) using microarray technology platforms such as described in U.S. Ser. No. 10/106,097, entitled “Methods for Genomic Analysis”, filed on Mar. 26, 2002, assigned to the same assignee as the present application; U.S. Ser. No. 10/284,444, entitled “Chromosome 21 SNPs, SNP Groups and SNP Patterns,” filed on Oct. 31, 2002, assigned to the same assignee as the present application; and Ser. No. 10/042,819, entitled “Whole Genome Scanning,” filed on Jan. 7, 2002, assigned to the same assignee as the present application, all of which are incorporated herein by reference. In one embodiment, the microarrays are manufactured using a process adapted from semiconductor manufacturing to achieve cost effectiveness and high quality.

Haplotype Structure Analysis

The polymorphisms identified may be grouped into haplotype blocks and haplotype patterns using methods disclosed in U.S. Ser. Nos. 10/106,097, entitled “Methods for Genomic Analysis”, filed Mar. 26, 2002 (Attorney Docket 200/1005-10), incorporated herein by reference. Representative polymorphisms, haplotype blocks and haplotype patterns from an entire human chromosome (chromosome 21) are disclosed in, for example, Patil, N. et al, “Blocks of Limited Haplotype Diversity Revealed by High-Resolution Scanning of Human Chromosome 21” Science 294, 1719-1723 (2001) and the associated supplemental materials, incorporated herein by reference.

Identification of a 200,000 SNP Marker Set for Individual Genotyping in a Whole Genome Association Study

It is beneficial to have adequate power to identify disease-influencing traits in association studies a set of dense SNP markers that are uniformly spaced and have high allele frequencies across all human populations. It has been demonstrated that SNP allelic variants within 10-25 kb of each other in the human genome are often highly correlated (Patil et al, 2001). A set of 200,000 SNPs is identified that contains greater than 80% of the genetic information of the ˜1.1 million SNPs across the genome in the population used to discover them. In one example, a 200,000 SNP marker set is comprised of SNPs distributed at an average density of 1 per 10 kb across the human genome and with allele frequencies greater than 10% in diverse populations. These SNPs are chosen based on their ability to represent a large fraction of the genetic information of the entire set of 1.1 million common SNPs. By using this subset of “informative SNPs” (or “haplotype tagging SNPs” or “tag SNPs”), comprehensive association studies maintaining the power of a 1.1 million SNP set but in a much more cost-effective manner may be performed. The reduction in cost allows a shift from genotyping pooled samples to genotyping individual samples.

Validation of the Set of SNPs in a Second Ethnically Diverse Population

A second diverse population is examined to determine the frequencies of the SNP alleles. If SNP markers are common across human populations, they can be used in association studies regardless of the ethnicities of the groups in the study. To determine the informative nature of SNPs in populations other than the one in which they were discovered, a set of SNPs is genotyped in a second group of ethnically-diverse individuals. In one example, only 20 (1%) of the SNPs show no allelic variation in any of the tested individuals, and an additional 15% have a minor allele frequency of less than 10%. Thus, 84% of the SNPs are common and informative in a separate collection of ethnically-diverse individuals, unrelated to the individuals used for SNP-discovery. The minor allele frequencies of these SNPs are fairly evenly distributed from 0.1 to 0.5 with a median of 0.25 (FIG. 3). The correlation of the 200,000 SNP marker set with other common SNPs that are variable in this second diverse population but that are not part of the 200,000 SNP marker set are empirically determined. This facilitates assessment of the utility of the 200,000 marker set for whole genome association studies in diverse populations other than the one in which the SNPs were discovered. Such an analysis reveals, for example, that the 200,000 SNP marker set is able to predict the genotypes for 64% of markers tested that were not part of the marker set with an average coefficient of determination R₂) of 0.85. This demonstrates the considerable power of the 200,000 SNP marker set for performing whole genome association studies in diverse populations.

Sample Collection

DNAs may be collected from case probands (targeted n=800) and unaffected siblings (targeted n=1,222). DNAs may also be collected from unrelated controls (targeted n=800). In preferred embodiments, DNA extraction and storage is performed via informed consent. In some embodiments, the median age of cases at onset (range) is 62 years (28-87), and the median age at study (range) is 68 years (30-98). In some embodiments, 62% of cases are men and 38% are women. In certain embodiments, characteristics comparable to those observed for a defined population (Bower et al. (2000) “Influence of strict, intermediate, and broad diagnostic criteria on the age- and sex-specific incidence of Parkinson's disease”, Mov Disord 15:819-825; Bower et al. (2003) “Head trauma preceding PD: A case-control study”, Neurology 60:1610-1615) may be used. In a particular embodiment, 19% of cases report PD in at least one first degree relative. In another particular embodiment, nearly all cases are Caucasian Americans of European ancestral origin.

An initial study sample may consist of DNAs obtained from, e.g., 500 casesibling pairs discordant for PD. These pairs represent 500 unrelated sibships, for which there is DNA available for both the case proband and at least one unaffected sibling. When there are DNAs available for multiple unaffected siblings, an attempt is made to match a sibling of the same gender as the proband (where able), and subsequently match for closest age to the proband (with a bias preference for the older unaffected sibling when there are two unaffected siblings equally close in age to the proband). A follow-up sample may consist of, e.g., 300 case-unrelated control pairs, matched for gender, age at study (plus or minus one year), self-reported ethnicity, and geographic residence (regions defined as the 120-mile radius surrounding the Mayo Clinic Rochester; the remainder of Minnesota, Wisconsin, and Iowa; and North and South Dakota combined). In one embodiment, the unrelated controls may be identified via random digit dialing (under age 65) and randomly from a list of Medicare recipients (Center for Medicare and Medicaid Services; ages 65 and older). The demographic and clinical characteristics for the subjects selected for this study are comparable to those described for the full available sample

Typically, 20 cc of venous blood is collected. From 5 cc, 100-200 mg of DNA is extracted per subject (Purgene procedure). Such DNA is of high quality and of high molecular weight, as assessed by spectrophotometer. The remainder of the blood (15 cc) is saved as a buffy coat, from which DNA can be extracted at a later data. The DNA samples are stored at −70 degrees centigrade.

Matching for Differences in Ancestry (Population Stratification) Between Cases and Controls

In light of possible stratification bias, a study design for a genetic association study using outbred populations must be carefully considered. A conservative sampling approach for late-onset disorders is to pair cases with their unaffected siblings. In the absence of non-paternity, these discordant sibling pairs are ethnically similar and hence this design eliminates the effects of stratification bias. Allele frequencies can then be compared via flexible adaptations of the transmission disequilibrium test (TDT) (Schaid and Rowland (1998) “Use of parents, sibs, and unrelated controls for detection of associations between genetic markers and disease”, Am J Hum Genet 63:1492-1506). However, the use of unaffected sibling controls may result in a loss of statistical power because of misclassification of asymptomatic carriers and because of over-matching for environment. Others have proposed the use of “genomic controls” (i.e., controls matched to cases for multiple loci unlikely to affect liability) (Devlin and Roeder (1999) “Genomic control for association studies”, Biometrics 55:997-1004; Bacanu et al. (2000) “The power of genomic control”, Am J Hum Genet 66:1933-1944). However, most genetic association studies of PD have employed unrelated controls matched to cases for age, gender, and ethnicity classified in broad groups (e.g. “Caucasians”, “Asians”, or “Chinese”).

For the study exemplified here, an available subset of 500 sibling pairs discordant for PD (one pair each from 500 unrelated sibships) is used for the primary screen, and 300 PD case-unrelated control pairs (matched for age at study, gender, self-reported ethnicity, and geographic residence) is used for a follow-up study sample. While the affected and unaffected groups for the primary screen (sibling pairs) are ethnically matched, stratification bias in the case and control groups used for the follow-up study is tested and corrected. The study of sibling pairs discordant for PD limits false positive genetic associations as a result of population stratification bias, and the additional study of unrelated case-control pairs optimizes statistical power for identifying significant genetic associations.

The use of discordant sib-pairs would limit the false positive genetic associations as a result of population stratification bias in the primary screen of the proposed study. However, such bias has to be considered for the replication study sample, which consists of case and control individuals. To address this issue, a process for detecting and correcting for ancestry differences between cases and controls may be utilized. Such a process is described, e.g., in U.S. patent application Ser. No. 10/427,696, filed Apr. 30, 2003, entitled “Method for Identifying Matched Groups”; U.S. patent application Ser. No. 60/497,771, filed Aug. 26, 2003, entitled “Matching Strategies for Genetic Association Studies in Structured Populations”; and PCT patent application Ser. No. US04/013577, filed Apr. 30, 2004, entitled “Method for Identifying Matched Groups.” Specifically, case and control individuals are genotyped for a set of 312 SNP markers distributed across the genome and the SNPs are tested for an excess of significant allele frequency differences between the cases and controls. If significant differences are observed, the individuals sharing similar genotypes are empirically clustered into “inferred ancestry” groups using the model-based clustering algorithm STRUCTURE (Pritchard et al. 2000). Subsequently, case and control pools are constructed that are matched for inferred ancestry content.

High-Density Oligonucleotide Array-Based SNP Genotyping

Many methods for SNP discovery and genotyping have been developed over the last decade as alternatives to direct DNA sequencing. Although there are many alternative genotyping methods, including gel-based assays for single-strand conformation polymorphism (SSCP), enzyme cleavage methods, mass spectrometry, allele specific PCR, single nucleotide primer extensions, oligonucleotide ligation and pyrosequencing, most are not rapidly scalable in a cost effective manner to be used across a large number of SNPs. High-density oligonucleotide arrays offer the ability to simultaneously process large numbers of SNPs using automated methods across multiple samples. Light directed photo-lithography in conjunction with chemical coupling directs the synthesis of oligonucleotides of specific DNA sequence in pre-determined positions on a glass surface. Such high-density oligonucleotide arrays may be used to accurately genotype DNA samples in a rapid and cost-effective manner. In one embodiment, a high-density oligonucleotide array design uses 80 features (25-meroligonucleotides) to query each SNP. The 80 features comprise 10 overlapping feature sets where each feature set includes 4 features specific for the reference allele (one perfect match feature and 3 mismatch features), and 4 similar features for the alternate allele (FIG. 3). By comparing the fluorescence intensities of perfect match features for the reference allele with those that are perfect matches for the alternate allele, the three possible SNP genotypes (homozygous reference, heterozygous, and homozygous alternate) can be distinguished.

In one aspect, a high-throughput SNP genotyping platform uses a PCR-based sample preparation process. Regions of the genome containing SNPs of interest are specifically amplified using multiplex (e.g., 78-plex) short-range PCR. The PCR products from each individual are pooled and labeled with biotin, to create target DNA. The target DNA is hybridized to the SNP genotyping high-density oligonucleotide arrays. After overnight hybridization, the arrays are washed, stained, and scanned, and fluorescence intensities are determined for all features on the array. In the exemplified study, all the features necessary to genotype the 200,000 SNPs are arrayed onto a series of six high-density arrays, requiring target DNA with a complexity of approximately 30,000 SNPs be used for hybridization.

Statistical Analysis and Power

Linkage analysis is an effective strategy for the discovery of single genes that cause Mendelian-transmitted diseases in rare families. For complex diseases that result from multiple interacting genetic and environmental factors, high-resolution whole genome studies are expected to provide unprecedented power to identify susceptibility genes of modest effect (Daly, et al. (2001) “High resolution haplotype structure in the human genome”, Nat Genet 29:229-232; Goldstein (2001) “Islands-of linkage disequilibrium”, Nat Genet 29:109-111; Johnson, et al. (2001) “Haplotype tagging for the identification of common disease genes”, Nat Genet 29:233-237; Uhl, et al. (2001) “Polysubstance abuse-vulnerability genes: genome scans for association, using 1,004 subjects and 1,494 single-nucleotide polymorphisms”, Am J Hum Genet 69:1290-1300; Uhl, et al. (2002) “Substance abuse vulnerability loci: converging genome scanning data”, Trends Genet 18:420-425). However, the success of this approach is contingent upon large sample size and genotyping of a very large number of markers. Because PD is a complex disorder, high-resolution whole genome approaches are well suited to its study. The large, well-characterized case-control samples, in combination with the proprietary 200,000 SNP marker set should provide sufficient statistical power to detect many susceptibility genes for PD.

The power to detect a genetic locus in an association study depends on several features of that locus as well as features of the study design. The frequency of the allele that confers increased risk, its “mode of inheritance” (whether the allele has a dominant, recessive, or additive effect), and the absolute magnitude of its effect are all important factors and generally unknown before a study is carried out. The number of loci contributing to a trait is also generally unknown. Important features of the study design include the numbers of “cases” and “controls” and whether or not they are related, the ranges of phenotypes in the cases and controls, and the acceptable false-positive and false-negative rates.

The power of the exemplary association study was calculated to identify genetic loci contributing to the PD phenotype based on an additive threshold model (Risch and Teng (1998) “The Relative Power of Family Based and Case-Control Designs for Linkage Disequilibrium Studies of Complex Human Diseases”, Genome Res 8(12):1273-1288). As the features of the PD-susceptibility loci are unknown, the following assumptions are made to define parameters for the power calculations: 1) A PD population prevalence of 1% 2) a sample size of 500 discordant sibpairs without parents, with a follow-up sample of 300 additional unrelated PD cases and 300 unrelated controls 3) the frequency of the minor allele of both the PD-susceptibility locus and the SNP marker to be 0.25 (based on the median minor allele frequencies of SNPs from Perlegen Sciences' SNP set genotyped in previous experiments—see Section C4) 4) an average coefficient of determination (R₂) between each PD susceptibility locus and the best-associated marker in the 200,000 SNP set of 0.64* 0.85=0.55 (see Section C4) and 5) a very conservative type I error rate, corrected for multiple comparisons, of 0.05/200,000=2.5×10₋₇. Using these parameters, the total exemplary study design has a power of greater than 80% for identifying each gene that contributes a 2.5 fold or greater risk of PD (Risch and Teng (1998), supra).

A cost-effective, two-tiered design is employed. Rather than genotyping all 200,000 SNPs in the total sample of 800 cases and 800 controls, first all 200,000 SNPs in just the 500 discordant sibpairs are genotyped, and those SNPs that show significant allele frequency differences with a type I error rate of less than 0.01 (i.e. 200,000 SNPs×0.01=2000 SNPs that will pass this significance threshold) are selected. This approach allows the identification of all 9 expected loci, each contributing 5% or greater of the PD phenotype, with greater than 98% power, using the additive model parameters described above. In order to distinguish the expected 9 true positive SNPs from this set of 2000 SNPs, only these 2000 SNPs are genotyped in the follow-up set of 300 PD cases and 300 controls. This results in an overall power of 80% as described above, while providing considerable cost savings by genotyping only 2000 SNPs, rather than the entire set of 200,000 SNPs, in the follow-up sample of 300 cases and 300 controls.

Specific Aim 1: Determine the Allele Frequency Differences of the 200,000 SNPs in 500 Sibpairs Discordant for PD

A first specific aim is to determine the SNP allele frequency differences for the approximately 200,000 SNPs between individuals with PD and their unaffected siblings. Specifically, each individual from the 500 discordant sibpairs (1000 individuals in total) are independently genotyped for all 200,000 informative SNPs using a SNP genotyping high-density oligonucleotide arrays. These genotyping data are analyzed to identify SNP markers with significant allele frequency differences between the PD affected and unaffected sibs. Such genotyping is rapid, cost-effective, and accurate.

1 μg of genomic DNA (in 10-20ul of Tris-EDTA) isolated from each individual is be provided in 96-well format. Each DNA sample is visually inspected for quality, and a set of 24 randomly-selected samples is subjected to the following quality controls: PCR amplification and gel analysis to examine DNA quality and a picogreen assay to verify the quantity. Each DNA sample is quantified by picogreen assay, and then amplified using Molecular Staging's multiple displacement amplification (MDA) technology. MDA is an isothermal, strand displacement amplification technology which amplifies the whole genome in a highly uniform manner (Dean, et al. (2002) “Comprehensive human genome amplification using multiple displacement amplification”, Proc Natl Acad USA 99:5261-5266; Hosono, et al. (2003) “Unbiased whole-genome amplification. directly from clinical samples”, Genome Res 13:954-964; Lage, et al. (2003) “Whole genome analysis of genetic alterations in small DNA samples using hyperbranched strand displacement amplification and array-CGH”, Genoome Res 13:294-307). The MDA-amplified genomes are tested for yield and quality as a PCR template, normalized by dilution, and then used as a template for short-range PCR. Methods for selection short-range PCR primer pairs are detailed, for example, in U.S. patent application Ser. No. 10/341,832, filed Jan. 14, 2003, “Apparatus and Methods for Selecting PCR Primer Pairs”.

Regions of the genome containing SNPs of interest are specifically amplified using 200,000 short-range PCR primers in a multiplexed (78-plex) manner. The PCR products are pooled into a single sample per individual, and biotinylated to create target DNA. The target DNA from each individual is hybridized to a series of SNP-genotyping high-density oligonucleotide arrays, together containing the features necessary for genotyping all 200,000 SNPs. After overnight hybridization, the arrays are washed, stained, and scanned. Fluorescence intensities are determined for all features on the array. The genotype of each SNP is determined by analyzing the resultant fluorescent patterns. Stringent criteria are used to evaluate the quality of each SNP call, based on the relative intensities of the perfect match and mismatch features (25-mer oligonucleotides tiled onto the array). Each sample is actively tracked throughout the process, from the receipt of the DNA to the scanning of the hybridized array and automated analysis of the fluorescence intensity data.

The scan files generated by the scanner are analyzed by software programs designed to interpret intensity data from microarrays. This software assigns genotypes at each SNP position for each individual -in the case and control groups. The data are analyzed according to the methods disclosed in the following U.S. patent applications, all of which are assigned to the assignee of the present applications: U.S. patent application Ser. No. 10/351,973, filed Jan. 27, 2003, entitled “Apparatus and Methods for Determining Individual Genotypes”; and U.S. patent application Ser. No. 10/786,475, filed Feb. 24, 2004, entitled “Improvements to Analysis Methods for Individual Genotyping”. The nucleic acids listed in Tables 1 and 2 were identified as strongly associated with the case or control group.

In particular, each SNP assayed is tested for significant allele frequency differences between affected and unaffected groups using χ² tests. Alternatively, or in addition, the S-TDT test (sib Transmission Disequilibrium Test) may be applied on the overall sample set and/or on a set of age and gender strata within the samples (e.g., males, females, age <66, age >66, etc.) Another test that may be applied is Conditional Logistic Regression using strata such as those used for the S-TDT test. Assuming an additive polygenic model and loci of equal contribution, ˜9 major genes, each accounting for ˜5% of the variability of the trait are predicted. In this initial screen, there is 98% power to detect all of the anticipated ˜9 major genes believed to define the genetic basis of PD, with each major gene accounting for 5% or more of the PD phenotype. To achieve this high level of power, a false positive rate of 0.01 is set. Thus, from an original set of 200,000 SNP alleles genotyped, 2,000 positive alleles are expected to be detected. In order to identify the ˜9 true PD-associated alleles from among these 2000 positive alleles, the 2,000 alleles are genotyped in an additional sample of 300 unrelated PD cases and 300 unrelated controls.

Specific Aim 2: Individually Genotype SNPs Putatively Associated with PD in an Unrelated Population

A second specific aim this exemplary study is to individually genotype SNPs with allelic differences in the original population in an unrelated population to identify the set of SNPs truly associated with PD. Given the type I error rate expected for the proposed number of SNP markers, a substantial fraction of the SNPs showing significant allele frequency differences between the affected and unaffected samples in Aim 1 are expected to be false positives. However, SNPs that are truly associated with PD should also have allele frequency differences in a second population of PD cases and controls. Thus, to identify the set of SNP markers that are truly associated with PD, the ˜2000 SNP markers showing the most significant allele frequency differences in the original population are selected and genotyped in a follow-up population (300 PD cases and 300 unrelated controls) using the same high-density oligonucleotide array technology.

Three hundred case and 300 unrelated controls are used as a follow-up population in which to reduce the association of false-positives detected in the primary screen (Aim 1). The case and control samples are matched for gender, age at study (plus or minus one year), self-reported ethnicity, and geographic residence (regions defined as the 120-mile radius surrounding the Mayo Clinic Rochester; the remainder of Minnesota, Wisconsin, and Iowa; and North and South Dakota combined).

To test for possible population stratification bias, high density array technology is used to genotype all individuals from the case and control groups, using a set of 312 SNP markers distributed across the genome and testing the SNPs for significant allele frequency differences (see section entitled “Matching for Differences in Ancestry (Population Stratification) Between Cases and Controls”). If significant differences are observed, individuals sharing similar genotypes are empirically clustered into “inferred ancestry” groups using the model-based clustering algorithm STRUCTURE (Pritchard, et al. (2000) “Inference of population structure using multilocus association genetic mapping studies of complex traits”, Bioinformatics 19:149-150). The two groups are matched for inferred ancestry, if necessary, by removing samples until the ethnic makeup of the two groups is similar.

The individual samples from the case and control groups are genotyped for the 2000 putatively-associated SNPs using the same procedures as in the initial screen (Specific Aim 1). The primers for PCR amplification of these 2000 SNPs are the same as were used to amplify the same SNPs in Aim 1. Thus, no new PCR primer development is needed for genotyping this replicate population. The 2,000 SNPs are analyzed, and those with significant allele frequency differences between affected and unaffected groups in both the primary and replicate populations are identified as being positively-associated with PD.

Statistical Analysis of Genotyping Results from the Replicate Population

A genotyping quality score may be determined from an independent set of SNPs that were genotyped on a shared set of samples with multiple technologies, allowing for a correlation of cross-platform discrepancies with internal data quality metrics. Bagged classification trees are used to build a model for the inter-platform discrepancy probability p. SNP genotypes with p<0.20 are selected from the genotypes of the replicate populations, which yields an estimated overall discrepancy rate in genotypes against other platforms of ˜1% in the reported genotypes. Further, quality control measures, which include checks for identity and call rates exceeding 80% of the assayed SNPs at the selected threshold of discrepancy probability p<0.20, are implemented.

Filters may be utilized to exclude SNPs with lower quality genotypes such as, for example, a genotyping call rate of >80% and a Hardy-Weinberg equilibrium p-value on controls >0.0001. These filters may exclude, for example, 2% of SNPs on a chip.

An initial analysis of differences between cases and controls may be based on Armitage trend tests for case/control status as a function of the number of alternate alleles (r=0, h=1, a=2). This coding implies an additive model for the combined effect of the two alleles in each individual on the phenotype. When the genotypes for a SNP are in perfect Hardy-Weinberg equilibrium, this test is equivalent to a chi-squared test performed on the 2×2 contingency table of allele counts; however, it accounts for the correlations between alleles that arise with deviations from Hardy-Weinberg equilibrium. The test statistic from the trend tests is distributed as χ²(1)

Differences between the distribution of ancestral populations in the cases and controls of an association study affect the observed distribution of allele frequency differences in the study. The effect of population structure can be modeled in several different ways. One such method is the Genomic Control method (see, e.g. “Genomic Control for Association Studies,” B. Devlin and K. Roeder, Biometrics, 1999, 55(4), p997). Under certain assumptions such as similar rates of mutation and recombination across different parts of the genome, the distribution of allele frequency differences in an association study subject to population structure can be modeled as an overall inflation of the chi-squared-distributed test statistics of a test such as the trend test that we have performed. The extent of inflation of the test statistics is determined from a measure such as the median of the distribution of test statistics, evaluated over an unlinked set of markers. All statistics are then scaled by this inflation factor, and the p-values for each SNP are evaluated thereafter. A set of 311 SNPs may be tiled for the purposes of exploring and correcting for population structure. However, all passing 3079 SNPs from the replication study may be chosen for use in the estimation of the Genomic Control inflation factor λ. Including a small number of SNPs that may be truly associated with the phenotype of interest should make the correction potentially a little conservative, but the much larger SNP set also greatly reduces the variance in the estimate of the correction required. In one such example, the inflation values that we deduced for the reported SNP set on the various strata of samples were:

| | Genomic Control |
|---|---|
| Samples | inflation λ |
| all | 1.068 |
| female | 1.101 |
| male | 1.000 |
| old | 1.079 |
| young | 1.039 |

These results do not indicate worrisome levels of population stratification. The GC approach should provide an adequate correction for this level of stratification. It should be noted that the GC approach doesn't explicitly model population structure; it only controls the false positive rate to be the same as would be observed in the absence of population structure. Other approaches, such as Pritchard's Structured Association test, address this problem in greater detail.

In the absence of true associations, the p-values from analyses. performed on the replication samples should be uniformly distributed between 0 and 1. Deviations from this expected null distribution can be evaluated in a number of ways, ranging from the conservative Bonferroni correction of significance thresholds (t=0.05 /n˜2.4e-5 for the replication SNPs excluding the GC SNPs) to methods that estimate the False Discovery Rate (FDR) in various ways, for example, using John Storey's QValue program to estimate the FDR for each SNP in our analyses.

It is also desirable to combine the significance information from both sets of samples. There are several methods in the meta-analysis literature for this sort of analysis. The simplest of these—Fisher's combined p-value—is based on independence between the tests being combined and does not account for possible discrepancies in the sign of the effects across the tests being combined. Other tests address these and other issues, but Fisher's combined p-value provides a starting point for such analyses. Also, it is possible to perform FDR-type analyses on the combined p-values in a manner that accounts for the biased set of SNPs assessed in the replication samples. In one embodiment, one such method is employed, in which the FDR is assessed within the set of SNPs in Category 1 (these had been selected for p_Overall<0.01).

A test may be performed that looks at consistency between the effect signs (independent of size) for the two sets of samples. A bias towards consistent effect directions is evaluated for significance. In one example, the effect signs are consistent in 1470 of the 2777 SNPs (excluding the GC SNPs) reported, yielding a p-value of 0.002 on a binomial test. However, looking closer at the SNPs with most significant p-values on the trend tests, there is no marked increase in consistency, and even for the most significant subset of SNPs effect directions are not consistently in agreement. This seems to indicate that if there are in fact several truly associated SNPs in the replication set, their effect sizes are not much larger than would be expected in a data set of this size due to sampling variance alone.

XI. One Embodiment of a Two-Tiered Study Design

Identification of Polymorphisms

The entire human genome was scanned to identify common polymorphisms (and others) using microarray technology platforms such as described in U.S. Ser. No. 10/106,097, entitled “Methods for Genomic Analysis”, filed on Mar. 26, 2002, assigned to the same assignee as the present application; U.S. Ser. No. 10/284,444, entitled “Chromosome 21 SNPs, SNP Groups and SNP Patterns,” filed on Oct. 31, 2002, assigned to the same assignee as the present application; and Ser. No. 10/042,819, entitled “Whole Genome Scanning,” filed on Jan. 7, 2002, assigned to the same assignee as the present application, all of which are incorporated herein by reference. The microarrays were manufactured using a process adapted from semiconductor manufacturing to achieve cost effectiveness and high quality.

Haplotype Structure Analysis

The polymorphisms identified were grouped into haplotype blocks and haplotype patterns using methods disclosed in U.S. Ser. Nos. 10/106,097, entitled “Methods for Genomic Analysis”, filed Mar. 26, 2002 (Attorney Docket 200/1005-10), incorporated herein by reference. “Haplotype tagging” SNPs (also termed “tag”, “informative” or “representative” SNPs), haplotype blocks and haplotype patterns from an entire human chromosome (chromosome 21) are disclosed in, for example, Patil, N. et al, “Blocks of Limited Haplotype Diversity Revealed by High-Resolution Scanning of Human Chromosome 21” Science 294, 1719-1723 (2001) and the associated supplemental materials, incorporated herein by reference.

Tier 1

For the first phase or tier of the study, 443 case-unaffected (“case-control”) sibling pairs that were discordant for Parkinson's disease were individually genotyped. This family-based case-control study design limited false positive results due to population stratification bias (Maraganore DM (2005) “Blood is thicker than water: the strengths of family-based case-control studies”, Neurol 64:408-409). Further, individual genotyping (in tier 1 and tier 2) allowed analyses adjusted or stratified for age or gender, factors that have been previously shown to influence heritability in Parkinson's disease (Rocca, et al. (2004) “Familial aggregation of Parkinson's disease: the Mayo Clinic Family Study”, Ann Neurol 56:495-502). In addition, the individual genotyping data will allow future studies of complex gene-gene and gene-environment interactions, or genotype-phenotype correlations, within the available dataset. Cases were enrolled prospectively from the clinical practice of the Department of Neurology of the Mayo Clinic in Rochester, Minn. from the time period June 1996 through May 2004. They all resided within Minnesota or one of the surrounding four states (Wisconsin, Iowa, South Dakota, or North Dakota). All cases underwent a standardized clinical assessment performed by a neurologist sub-specialized in movement disorders. Table 3 in Appendix 1 summarizes demographic and clinical characteristics for the samples. Cases had at least two of four cardinal signs of parkinsonism (rest tremor, rigidity, bradykinesia, or postural instability) and no features atypical for Parkinson's disease (such as unexplained upper motor neuron or cerebellar signs). When non-motor manifestations such as dysautonomia or dementia were present, they were mild and occurred late in the disease course. Secondary causes of parkinsonism (e.g., history of neuroleptic exposure, encephalitis or multiple strokes) were excluded. All patients treated with a gram total a day or more of levodopa (in combination with carbidopa) had a more than minimal improvement in parkinsonism symptoms and signs. A genealogical history was obtained from all cases, and when permitted available siblings were contacted for a telephone interview to exclude parkinsonism via a validated screening instrument (see, e.g., Rocca W A, Maraganore D M, McDonnell S K, Schaid D J; “Validation of a telephone questionnaire for Parkinson's disease”; J Clin Epidemiol 1998;51:517-523). For cases and siblings screening positive for Parkinson's disease, blood was obtained following a clinical assessment performed either at the Mayo Clinic or in the subjects+ homes. For siblings screening negative for Parkinson's disease (i.e., none of the following: prior diagnosis of Parkinson's disease, treatment with levodopa, or three or more of 9 symptoms), blood was obtained via mail in kits (no clinical assessment was performed). Cases were matched to a single participating sibling without Parkinson's disease or parkinsonism, of the same gender (when able) and then of closest age at the time of the study. All subjects provided informed written consent and whole blood was obtained via venipuncture for DNA extraction (via the Puregene method, Gentra Sciences) and storage. Approximately one microgram of DNA for each subject (matched discordant sibling pairs) was provided for laboratory study including amplification and genotyping.

Whole genome amplification was performed as previously described (see, e.g., Dean FB, Hosono S, Fang L, Wu X, Faruqi A F, Bray-Ward P, Sun Z, Zong Q, Du Y, Du J, Driscoll M, Song W, Kingsmore S F, Egholm M, Lasken R S “Comprehensive human genome amplification using multiple displacement amplification”; Proc Natl Acad Sci USA 2002;99:5261-5266). For each subject, DNA was individually genotyped for a set of 248,535 single nucleotide polymorphisms (SNPs) with unique positions on the NCBI human genome sequence (build 34) that were selected to have relatively uniform spacing across the genome. These SNPs occur with a 10% frequency or greater in multiple populations and were previously found to capture via linkage disequilibrium a large fraction of all common human DNA variation and so were considered to be haplotype tagging/defining SNPs, or “tag SNPs” (see Hinds D A, Stuve L L, Nilsen G B, Halperin E, Eskin E, Ballinger D G, Frazer K A, Cox D R “Whole-genome patterns of common DNA variation in three human populations”; Science 2005; 307:1072-1079; and Patil, et al. (2001) “Blocks of Limited Haplotype Diversity Revealed by High-Resolution Scanning of Human Chromosome 21”, Science 294:1719-1723). The genotyping platform employed high-density oligonucleotide arrays such that one hybridization yielded genotypes for 85,000 SNPs in a single individual. A liberalization of the sibling transmission disequilibrium test (sTDT) (Schaid, DJ, Rowland, C (1998) “Use of parents, sibs and unrelated controls for detection of associations between genetic markers and disease”, Am J Hum Genet 63:1492-1506) was performed to identify SNPs that had significant allele frequency differences in cases versus unaffected siblings, adjusting the analyses for age and gender (Spielman R S, Ewens W J “A sibship test for linkage in the presence of association: the sib transmission/disequilibrium test”; Am J Hum Genet 1998;62:450-458). For each SNP we calculated odds ratios, 95% confidence intervals (95% CI), and p values (using a log additive model).

Tier 2

For tiers 2a and 2b of the study, 332 matched case-unrelated control pairs were individually genotyped. This case-unrelated control study design optimized statistical power for the replication study (Maraganore DM (2005) “Blood is thicker than water: the strengths of family-based case-control studies”, Neurol 64:408-409). Cases were enrolled as for tier 1 (see Table 3 in Appendix 1), but had no siblings available. Unrelated controls were identified via random digit dialing from the same 5-state region as the cases and were screened negative for parkinsonism via the same validated telephone instrument as for siblings. DNA was collected at the time of clinical assessment for cases, and via mail in kits for controls. Case-unrelated control pairs were matched for gender and age (+/−3 years).

For tier 2a, a subset (1,518) of SNPs that were associated with Parkinson's disease (p<0.01 in tier 1) were genotyped, using customized oligonucleotide arrays. An additional pre-defined set of 311 SNPs were genotyped for genomic control (Hinds DA, Stokowski R P, Patil N, Konvicka K, Kershenobich D, Cox D R, Ballinger D G “Matching strategies for genetic association studies in structured populations”; Am J Hum Genet 2004;74:317-325). Conditional logistic regression analyses were performed to test for and model associations between the SNPs and Parkinson's disease. The analyses were adjusted for age and gender as appropriate. Odds ratios, 95% confidence intervals, and p values were calculated as for tier 1. Population structure was also analyzed using the genomic control method (Devlin B, Roeder K “Genomic control for association studies”; Biometrics 1999;55:997-1004; Bacanu S-A, Devlin B, Roeder K “The power of genomic control”; Am J Hum Genet 2000;66:1933-1944; and Devlin B, Bacanu S, Roeder K (2004) “Genomic Control to the extreme”, Nature Genetics 36:1129-1130). This method treats the subjects as unmatched, and does not include adjustments for covariates; however, it should still provide guidelines for assessing the -degree of stratification in tier 2. For the SNPs associated with Parkinson's disease (p<0.01) in both the tier 1 and tier 2a analyses, data for the case-unaffected sibling and case-unrelated control pairs was pooled and the pooled data was again analyzed using a liberalization of the sTDT (Schaid, D J, Rowland, C (1998) “Use of parents, sibs and unrelated controls for detection of associations between genetic markers and disease”, Am J Hum Genet 63:1492-1506). This allowed maximization of the statistical power for the analyses so as to obtain a more precise ranking of replicated SNPs by ascending p values and a more precise estimate of odds ratios and 95% confidence intervals.

For tier 2b, additional SNPs were selected for genotyping that were only borderline significant in tier 1 (p<0.05) but that tested a priori biological or genetic hypotheses regarding genomic susceptibility to Parkinson's disease (n=1,312 SNPs). These SNPs did not achieve a p-value<0.01 in the tier 1 sample overall. However, some of these SNPs had p-values less than 0.001 in tier 1 strata defined by gender or by median age at study; or had p-values less than 0.05 and were positioned within +/−10 kb of the linkage-derived candidate genes alpha-synuclein, parkin, ubiquitin carboxy-terminal hydrolase LI, microtubule-associated protein tau, oncogene DJ1, and PTEN-induced kinase 1; or had p-values less than 0.05 and were positioned within additional loci linked to Parkinson's disease (i.e., PARK3, PARK8, PARK9, PARK10, PARK11); or had p-values less than 0.05 and were positioned within exons or within 10 kb 5′ of the transcript, genome-wide; or were redundant tags of Caucasian linkage disequilibrium bins (Hinds D A, Stuve L L, Nilsen G B, Halperin E, Eskin E, Ballinger D G, Frazer K A, Cox D R “Whole-genome patterns of common DNA variation in three human populations”; Science 2005; 307:1072-1079) highlighted by some of the above criteria. (The tier 2b SNP selection predated the discovery of mutations in the leucine-rich repeat kinase 2 gene as the cause of PARK8-linked parkinsonism, although SNPs within the gene were indeed selected and genotyped.) The association of these tier 2b SNPs with Parkinson's disease in the combined samples was also tested using a liberalization of the sTDT (as above).

Genotyping was performed using photolithographic microarrays (see Hinds D A, Stuve L L, Nilsen G B, Halperin E, Eskin E, Ballinger D G, Frazer K A, Cox D R “Whole-genome patterns of common DNA variation in three human populations”; Science 2005; 307:1072-1079).

Data Analysis

For tier 1,443 case-unaffected sibling pairs were genotyped. Table 3 summarizes demographic and clinical characteristics for the sample. For the 248,535. SNPs selected, the genotyping call rate was greater than 80% for each of 220,143 SNPs. Of these SNPs, 205,031 (93%) were polymorphic within the study sample. The Hardy-Weinberg equilibrium (HWE) p value was greater than 0.001 for 198,345 SNPs (97% of polymorphic SNPs). Ultimately, for these subjects and SNPs, 175,420,019 genotype calls were made (98.1% of possible genotypes). There were 96 SNPs that were re-genotyped in triplicate for each subject, with 99.8% concordance of genotypes. The concordance of genotypes called by the oligonucleotide array platform as compared to genotypes called by other platforms employed as part of the multi-center HapMap project was greater than 99.5% (Hinds D A, Stuve L L, Nilsen G B, Halperin E, Eskin E, Ballinger D G, Frazer K A, Cox D R “Whole-genome patterns of common DNA variation in three human populations”; Science 2005; 307:1072-1079).

As a sensitivity analysis, the statistical power to detect unassayed, disease-associated variants with this SNP collection was determined. Specifically, these same SNPs were previously genotyped in a different sample of European Americans that were also sequenced across selected genes by the Seattle SNP Program for Genomic Applications (PGA) (SeattleSNPs. NHLBI Program for Genomic Applications, UW-FHCRC, Seattle, Wash. [http://pga.gs.uwashington.edu]) (Hinds: D A, Stuve L L, Nilsen G B, Halperin E, Eskin E, Ballinger D G, Frazer K A, Cox D R “Whole-genome patterns of common DNA variation in three human populations”; Science 2005; 307:1072-1079). The metric of coverage was the mean r² value for any common PGA SNP (minor allele frequency>10%) with the most-correlated SNP in the same region from the working set of 198,345 SNPs. The statistical power to detect an unassayed, disease-associated allele indirectly using a correlated allele of an assayed SNP is related to r². Specifically, the power to detect an association indirectly in N samples is equivalent to the power to detect it directly in Nr² samples (Pritchard and Przeworski (2001) “Linkage disequilibrium in humans: models and data”, Am J Hum Genet 69:1-14). In spite the fact that only 3.3% of all of the common SNPs in these intervals (and only 1% of the less common SNPs) were assayed, the mean r² for unassayed SNPs was 0.57. By these measures, an effective sample size for unassayed SNPs of about 250 cases and 250 controls was achieved in this study. There were a total of 1,518 SNPs that were associated with Parkinson's disease in tier 1 (p<0.01).

For tier 2a, 332 case-unrelated control pairs were genotyped. Table 3 summarizes demographic and clinical characteristics for the sample. Genotypes and analyses were attempted for the 1,518 SNPs selected in tier 1, and for 311 genomic control SNPs. Of these, genotyping call rates of >80% and Hardy-Weinberg equilibrium p-values >0.0001 were achieved for 1,466 SNPs. 962,272 (98.9% of possible) genotypes were successfully called for these SNPs. To assess the impact of population structure on tier 2 associations, trend tests were performed on the genomic control SNPs. The mean trend test statistic over the genomic control SNPs, Xm, was 0.96, which is less than the expected value of 1 for no population structure (Devlin B, Bacanu S, Roeder K (2004) “Genomic Control to the extreme”, Nature Genetics 36:1129-1130). Therefore, there was no .indication of substantial confounding due to population structure bias.

There were 25 SNPs with p-values less than 0.01 in both tier 1 and tier 2a (termed replicated SNPs). Nine of these 25 replicated SNPs were genic, and 16 were intergenic, suggesting that intergenic sequences are also important to health, perhaps via gene regulatory effects (Martens, et al. (2004) “Intergenic transcription is required to repress the Saccharomyces cerevisiae SER3 gene”, Nature 429:571-574). After calculating p-values for the tier 1 and tier 2a data combined, only 11 of the SNPs had nominal p-values less than 0.01 and the same direction of the effect in tier 1 and tier 2a. A SNP within the SEMA5A gene had the lowest combined p-value (OR=1.74; 95% CI 1.36-2.24; p=7.62 ×10⁻⁶). A second SNP tagged the PARK 11late onset Parkinson's disease susceptibility locus (OR =1.84; 95% CI 1.38 2.45; p=1.70×10^{m-5}). Table 4 in Appendix 1 lists the 11 replicated SNPs with combined p-values less than 0.01, including their dbSNP reference sequence identification numbers, and where applicable the corresponding gene ontology annotation of nearby genes. The SNPs are ordered by ascending p-values.

Table 5 (categories 2-7) summarizes the rationale for inclusion of the tier 2b SNPs based primarily on a priori biological or genetic hypotheses regarding susceptibility for Parkinson's disease. 1,312 tier 2b SNPs (842,616 additional genotypes —98.8% of possible) were considered. Of these, the SNP with the lowest combined p-value tagged the PARK10 late onset Parkinson's disease susceptibility locus (rs682705; OR=1.53; 95% CI 1.26-1.85; p=9.07×10⁻⁶).

Discussion

Twin studies and familial aggregation studies have suggested that the genetic contribution to Parkinson's disease is small and limited to younger onset cases (Wirdefeldt et al., No evidence for heritability of Parkinson's disease in Swedish twins. Neurology 2004;63:305-311; Rocca et al., Familial aggregation of Parkinson's disease: the Mayo Clinic Family Study. Ann Neurol 2004;56:495-502). However, parametric linkage studies have assigned 11. loci to Parkinson's disease, and causal mutations have been identified in six genes (alpha-synuclein, parkin, ubiquitin carboxy-terminal hydrolase L1, oncogene DJ1, PTEN-induced kinase 1, and leucine-rich repeat kinase 2) (Polymeropoulos, et al., Mutation in the alpha-synuclein gene identified in families with Parkinson's disease. Science 1997;276:2045-20477; Kitada et al., Mutations in the parkin gene cause autosomal recessive juvenile parkinsonism. Nature 1998;392:605-608; Leroy et al., The ubiquitin pathway in Parkinson's disease. Nature 1998;395:451-452; Bonifati et al., Mutations in the DJ1 gene associated with autosomal recessive early-onset parkinsonism. Science 2003;299:256-259; Valente et al., Hereditary early-onset Parkinson's disease caused by mutations in PINK1. Science 2004;304:1158-1160; Zimprich et al., Mutations in LRRKS cause autosomal-dominant parkinsonism with pleomorphic pathology. Neuron 2004;44:575-577). Non-parametric linkage studies have also assigned the locus containing the microtubule-associated protein tau gene to Parkinson's disease (Martin et al., Association of single-nucleotide polymorphisms of the tau gene with late-onset Parkinson's disease. JAMA 2001;286:2245-2250). While mutations in these genes only rarely cause Parkinson's disease, common variations in some of these genes may confer susceptibility with a more sizeable population attributable risk (Farrer et al. (2001) “Lewy bodies and parkinsonism in families with parkin mutations”, Ann Neurol 50(3):293-300; Farrer et al. (2001) “alpha-Synuclein gene haplotypes are associated with Parkinson's disease”, Hum Mol Genet 10(17):1847-51; West et al., Functional association of the parkin gene promoter with idiopathic Parkinson's disease. Hum Mol Genet 2002;1 1:2787-2792; Maraganore et al., Ann Neurol 2004;55:512-521; Mamah et al., Interaction of α-synuclein and tau genotypes in Parkinson's disease. Ann Neurol (in press)). However, the search for Parkinson's disease susceptibility genes has been largely limited to the candidate genes approach, which has been mostly non-informative.

In this study, common Parkinson's disease susceptibility gene variants were identified via a whole-genome association design. Despite the low heritability of Parkinson's disease, several new susceptibility genes for Parkinson's disease (Table 4) have been nominated. Findings for the SEMA5A gene are particularly noteworthy and highlight the apoptosis pathway (Tatton et al., Apoptosis in Parkinson's disease: signals for neuronal degeneration. Ann Neurol 2003;53:S61-S72). Although the effect size is small (OR=1.7; 95% CI 1.36 - 2.24), the disease-associated allele occurs with sufficient frequency to confer a sizeable population attributable risk (minor allele frequency=19.6%, unrelated controls).

The results of this study may be used to determine biomarkers for early disease detection or to identify new molecular targets for disease-modifying therapies (secondary prevention), and may contribute to primary prevention strategies. Further, association studies determining genotypes related to prognostic outcomes may predict the effect of treatments targeting these genes and their proteins (“virtual clinical trials”), and thus reduce research and development costs and identify subgroups of patients most likely to benefit from treatment (personalized medicine).

It is to be understood that the above description is intended to be illustrative and not restrictive. It readily should be apparent to one skilled in the art that various embodiments and modifications may be made to the invention disclosed in this application without departing from the scope and spirit of the invention. The scope of the invention should, therefore, be determined not with reference to the above description, but should instead be determined with reference to the appended claims, along with the full scope of equivalents to which such claims are entitled. All publications mentioned herein are cited for the purpose of describing and disclosing reagents, methodologies and concepts that may be used in connection with the present invention. Nothing herein is to be construed as an admission that these references are prior art in relation to the inventions described herein. Throughout the disclosure various patents, patent applications and publications are referenced. Unless otherwise indicated, each is incorporated by reference in its entirety for all purposes.

TABLE 3
Demographic and clinical characteristics (tiers 1 and 2)

| | Tier 1 | | Tier 2 | |
|---|---|---|---|---|
| General characteristics | PD Cases | Siblings | PD Cases | Controls |
| Total sample, n (%) | 443 (100) | 443 (100) | 332 (100) | 332 (100) |
| Men | 271 (61.2) | 214 (48.3) | 194 (58.4) | 193 (58.1) |
| Women | 172 (38.8) | 229 (51.7) | 138 (41.6) | 139 (41.9) |
| Age, median (range) | | | | |
| Age at onset | 61 (31-94) | — | 63 (36-88) | — |
| Age at study | 68 (33-96) | 66 (29-90) | 68 (42-90) | 67 (42-91) |
| Family history of PD, %^{a} | 20.5% | — | 14.9% | — |
| Region of origin of parents, n (%) | | | | |
| Both parents of European origin | 381 (86.0) | 363 (81.9) | 269 (81.0) | 272 (81.9) |
| Both parents Northern European^{b} | 111 (29.1) | 100 (27.5) | 84 (31.2) | 83 (30.5) |
| Both parents Central European^{c} | 145 (38.1) | 135 (37.2) | 96 (35.7) | 84 (30.9) |
| Both parents Southern European^{d} | 3 (0.8) | 3 (0.8) | 2 (0.7) | 3 (1.1) |
| Both parents European, mixed region | 122 (32.0) | 125 (34.4) | 87 (32.3) | 102 (37.5) |
| Only one parent of European origin^{e} | 39 (8.8) | 39 (8.8) | 43 (13.0) | 35 (10.5) |
| One parent declared “American”^{f} | 2 (0.5) | 1 (0.2) | 1 (0.3) | 4 (1.2) |
| Both parents declared “American” | 18 (4.1) | 23 (5.2) | 12 (3.6) | 19 (5.7) |
| Both parents Asian | — | 1 (0.2) | 1 (0.3) | — |
| Unknown | 3 (0.7) | 16 (3.6) | 6 (1.8) | 2 (0.6) |

^{a}Family history was defined as at least one affected first-degree relative; for tier 1, 90/439 cases had a family history of PD (missing = 4); for tier 2, 48/322 cases had a family history of PD (missing = 10).
^{a}“Northern European” includes Scandinavian, Swedish, Norwegian, Finnish, Danish, Irish, or British origins.
^{b}“Central European” includes French, Belgian, Dutch, Swiss, Luxemburgian, German, Austrian, Hungarian, Polish, Czechoslovakian, or Russian origins.
^{c}“Southern European” includes Italian, Spanish, Portuguese, Greek, or Yugoslavian origins.
^{d}Includes subjects for whom origin of one parent is unknown.
^{e}These subjects were all Caucasians and not Native Americans.

TABLE 2

| | | | | | | | |
|---|---|---|---|---|---|---|---|
| Ref snp_{—} | Ref Snp_{—} | Ref | Alt | Chromo- | Sex- | Accession | Contig |

| | | | | | | | | | | |
|---|---|---|---|---|---|---|---|---|---|---|
| SNP_ID | rsID | ssID | Allele | Allele | some | linked | ID | Position | Strand | Assayed sequence |

| | | | | | | | | | | |
|---|---|---|---|---|---|---|---|---|---|---|
| 651340 | 2245218 | 23835843 | C | T | 1 | A | NC_000001.5 | 13502523 | + | CCGGAGCCTTGGTCNTCGGTAT |
| | | | | | | | | | | TTAACAT |
| 1032590 | 682705 | 23154909 | A | G | 1 | A | NC_000001.5 | 54007335 | + | ACAACACGCAGTGGNCATCATC |
| | | | | | | | | | | AGAGGAA |
| 1032596 | 7520966 | 23154947 | C | T | 1 | A | NC_000001.5 | 54015180 | + | AGGGAACATAACAGNATAGCCC |
| | | | | | | | | | | ATCCTGT |
| 1128431 | 4147593 | 24245273 | T | A | 1 | A | NC_000001.5 | 162787644 | + | GAGTAGATATATGTNTTTGAGA |
| | | | | | | | | | | ATGAGAG |
| 1146860 | 1128952 | 23686700 | C | T | 1 | A | NC_000001.5 | 176304501 | + | AAATTTTTGATTTTNCTAAAAT |
| | | | | | | | | | | CCTTGGC |
| 719978 | 10779426 | 23186060 | A | T | 1 | A | NC_000001.5 | 218379723 | + | TAGGTAGTGGGGAGNCAAAGGT |
| | | | | | | | | | | AGCTTAG |
| 719775 | | | T | C | 1 | A | NC_000001.5 | 218521453 | + | GAAAGTTTGGAAAANTTAATCA |
| | | | | | | | | | | AAACTTA |
| 719756 | | | T | C | 1 | A | NC_000001.5 | 218529200 | + | TGCCATATCTAAACNTCAAAAT |
| | | | | | | | | | | TTCTTAA |
| 1225958 | | | A | G | 2 | A | NC_000002.6 | 11699188 | + | AGGTGGCAAGCTTCNTATTGGT |
| | | | | | | | | | | AAGAATT |
| 754013 | | | T | C | 2 | A | NC_000002.6 | 32459104 | + | CTTATTTTCTTGCANATTCAAT |
| | | | | | | | | | | GTAAGTT |
| 1270180 | 13016812 | 23861776 | A | G | 2 | A | NC_000002.6 | 67598836 | + | ACGCGGACCTGACTNCTACCCG |
| | | | | | | | | | | AACAACC |
| 1276993 | | | G | C | 2 | A | NC_000002.6 | 80767709 | + | TTTTCATGTGTCCTNCTAGCTA |
| | | | | | | | | | | GTTTTTT |
| 1367383 | 16851009 | 23902286 | C | T | 2 | A | NC_000002.6 | 166833251 | + | CATCAACCTGTGTTNACCTCCT |
| | | | | | | | | | | TTCTGTT |
| 1427562 | 6720502 | 24165630 | T | C | 2 | A | NC_000002.6 | 225588397 | + | AATATTTTTAATAGNCATTATG |
| | | | | | | | | | | TTACAGA |
| 1427591 | 13013735 | 24165672 | C | T | 2 | A | NC_000002.6 | 225609523 | + | GATATAGCTTTTCTNGCATTTT |
| | | | | | | | | | | TAAAAAT |
| 1431787 | 10200894 | 23902028 | C | G | 2 | A | NC_000002.6 | 229019671 | + | TCTTATACACTTTCNAATATGC |
| | | | | | | | | | | TGTACTA |
| 726185 | 10490012 | 23896593 | C | T | 2 | A | NC_000002.6 | 235130245 | + | ACTCATTTCTGCTCNTATTGTG |
| | | | | | | | | | | TTTTTAG |
| 4612764 | 2317421 | 23227649 | A | G | 2 | A | NC_000002.6 | 237454681 | + | GAGCAAAGGAGCTTNGCTCTTC |
| | | | | | | | | | | CATGCCA |
| 2329698 | 779708 | 24285691 | G | A | 3 | A | NC_000003.6 | 7487728 | + | AACATTCCTGAACCNCAATTCC |
| | | | | | | | | | | CTTCTTT |
| 2347338 | | | C | T | 3 | A | NC_000003.6 | 28058788 | + | GTCTCAGTGTAGCTNGTCAACC |
| | | | | | | | | | | CTGAAGT |
| 3255097 | 11919248 | 24270925 | A | G | 3 | A | NC_000003.6 | 30100697 | + | ACTTTGACAGTCACNCAGTCTG |
| | | | | | | | | | | CTTTCTA |
| 2382631 | 1669215 | 23911668 | T | C | 3 | A | NC_000003.6 | 77670329 | + | AATGCTCTACTCGANTGAAAAA |
| | | | | | | | | | | GACTTTT |
| 2503679 | 1000291 | 24350558 | G | A | 3 | A | NC_000003.6 | 190303721 | + | CAGCAAATAGTGAGNAAAGTTG |
| | | | | | | | | | | AATCTTA |
| 3393421 | 17719492 | 24395156 | C | G | 4 | A | NC_000004.6 | 344051 | + | CTATGCCATATGTTNCGATATG |
| | | | | | | | | | | TTCTATG |
| 3394795 | 16836832 | 23359882 | A | G | 4 | A | NC_000004.6 | 5271059 | + | GTTTTAAGCAAAATNTAGTCTC |
| | | | | | | | | | | AGCTCTG |
| 3492068 | | | C | T | 4 | A | NC_000004.6 | 72058145 | + | AATCCCTGGAAAAGNAGAATGT |
| | | | | | | | | | | ACCCGAT |
| 3414002 | 7694392 | 23265602 | C | T | 4 | A | NC_000004.6 | 103281820 | + | GAAATTATCTTCCTNGTGATCA |
| | | | | | | | | | | TTATCTA |
| 3413996 | 2631255 | 23265757 | G | A | 4 | A | NC_000004.6 | 103308314 | + | CAGTATTGGACCACNTCGCTAA |
| | | | | | | | | | | ATAAGCC |
| 3413959 | 2631271 | 23266345 | A | G | 4 | A | NC_000004.6 | 103372790 | + | TGGGAAGTAATTCTNAAACAGG |
| | | | | | | | | | | AGGCCTA |
| 2651858 | 1469259 | 24178620 | T | C | 4 | A | NC_000004.6 | 117844485 | + | ACCAAATTTTGCCANGTACTGA |
| | | | | | | | | | | CTTGGCA |
| 3526320 | 11737074 | 24402588 | G | A | 4 | A | NC_000004.6 | 125540194 | + | GTAGAAGTAGTAACNCACAGGC |
| | | | | | | | | | | ATAACAA |
| 2812059 | 1509269 | 23403866 | T | C | 4 | A | NC_000004.6 | 139331351 | + | AATTCAATCTCAAGNCCATCTG |
| | | | | | | | | | | GCTCCTG |
| 3025726 | 2313982 | 23404066 | A | G | 4 | A | NC_000004.6 | 139365687 | + | CCATAGTGCTATTCNTGTGTTT |
| | | | | | | | | | | TCAGTCC |
| 4040922 | 6827032 | 24633770 | G | C | 4 | A | NC_000004.6 | 154283058 | + | TTCCTTGATCTTTANTAGCTTT |
| | | | | | | | | | | TGGCAGT |
| 4417601 | 4691911 | 24659329 | T | A | 4 | A | NC_000004.6 | 164852476 | + | TTTTTCTTCTCTCANTCTCACC |
| | | | | | | | | | | ATGGACA |
| 4496903 | 1451213 | 23657057 | A | G | 4 | A | NC_000004.6 | 191137918 | + | CGCCATTGTTTTAANCACTATC |
| | | | | | | | | | | CTACTTT |
| 3113927 | 7723605 | 23884668 | T | C | 5 | A | NC_000005.5 | 5407353 | + | GGCTAAACGTGACTNGAAGTTT |
| | | | | | | | | | | CAGTGTA |
| 829480 | 7702187 | 23657222 | A | T | 5 | A | NC_000005.5 | 9385019 | + | GCAGTATGGACTGGNGCTTACA |
| | | | | | | | | | | TCATGTC |
| 840738 | 152562 | 24656498 | C | T | 5 | A | NC_000005.5 | 106917999 | + | CTCTTGAGCACTTANGCTTTAT |
| | | | | | | | | | | TTGTGTA |
| 3314438 | 17651424 | 24675503 | T | C | 5 | A | NC_000005.5 | 111564816 | + | TGTATGAATATGGANGCAATAT |
| | | | | | | | | | | AATAACA |
| 3336262 | 3213097 | 23489555 | A | T | 5 | A | NC_000005.5 | 158729574 | + | CTGATTATGTCTTTNTGCACTT |
| | | | | | | | | | | GGAGAAA |
| 1481740 | 12202337 | 24421639 | T | C | 6 | A | NC_000006.6 | 23795723 | + | GCCTTAGCAAATGANAGTAATT |
| | | | | | | | | | | CTGAAAA |
| 1481718 | 12205517 | 24421737 | T | C | 6 | A | NC_000006.6 | 23802517 | + | AATGGTCAGAAAAGNCAGTTTA |
| | | | | | | | | | | GTCCATT |
| 1481380 | 12192397 | 24423585 | G | A | 6 | A | NC_000006.6 | 23958782 | + | AAAAACTAAACTTTNAACTGAT |
| | | | | | | | | | | TCTGAGC |
| 1500751 | 7743819 | 23364906 | G | A | 6 | A | NC_000006.6 | 71102255 | + | TTGAAGATGAGAGTNCTAAAAA |
| | | | | | | | | | | TCTGTGA |
| 1522974 | 6904910 | 23383789 | T | C | 6 | A | NC_000006.6 | 113935407 | + | ATTGAGGACATACANAGTTTAT |
| | | | | | | | | | | GCTAGAA |
| 362893 | | | A | G | 6 | A | NC_000006.6 | 133540882 | + | GTTAACAATATGCTNAAGTATG |
| | | | | | | | | | | AATTTTT |
| 4355868 | | | G | A | 6 | A | NC_000006.6 | 162327821 | + | ATTTCTTTTCCCCANTCACCTA |
| | | | | | | | | | | ACTCAGT |
| 1549117 | 7799635 | 23520596 | A | G | 7 | A | NC_000007.8 | 6259487 | + | AGGTATGAACCCACNACTACAG |
| | | | | | | | | | | CTGGTCT |
| 1555513 | 1800795 | 24395511 | C | G | 7 | A | NC_000007.8 | 22508917 | + | TAGTTGTGTCTTGCNATGCTAA |
| | | | | | | | | | | AGGACGT |
| 535278 | 17148510 | 23415515 | T | A | 7 | A | NC_000007.8 | 23698145 | + | AGTCGTCCTTGTCGNAGGAGTT |
| | | | | | | | | | | TCAGGAA |
| 558560 | 17329669 | 24436780 | A | G | 7 | A | NC_000007.8 | 36593076 | + | TATATCTCCCATGGNTCAATTA |
| | | | | | | | | | | GGTGTTG |
| 567970 | 17172040 | 23378070 | T | C | 7 | A | NC_000007.8 | 42105704 | + | GATGTAAGTAACTCNAGAGCAT |
| | | | | | | | | | | AGGAAAT |
| 1577305 | 10499882 | 24066823 | T | C | 7 | A | NC_000007.8 | 84116696 | + | CAATAACTTTCCAGNGATGCCA |
| | | | | | | | | | | ATAATCC |
| 532112 | 10952539 | 23370649 | A | G | 7 | A | NC_000007.8 | 142016288 | + | AACAGGTCATGATGNTTCAGAA |
| | | | | | | | | | | AAGCAAA |
| 1590687 | 17382409 | 24444651 | A | G | 7 | A | NC_000007.8 | 142679898 | + | GCCTTGAAGGAGAANGCCACAG |
| | | | | | | | | | | GGCACGG |
| 1903046 | 16887478 | 23457423 | G | A | 8 | A | NC_000008.6 | 38459411 | + | ATTATTTGATGTCANCTTAGCA |
| | | | | | | | | | | TTATCAA |
| 1953706 | 16915399 | 24039581 | T | C | 8 | A | NC_000008.6 | 93934019 | + | CATTGGGGCTCCTCNAGCAATA |
| | | | | | | | | | | AATTGCT |
| 1953958 | 16915707 | 24040739 | C | T | 8 | A | NC_000008.6 | 94145054 | + | ACCAAGGAGCTGGTNCCATAGT |
| | | | | | | | | | | GGATTGT |
| 1980116 | 723268 | 23772641 | T | C | 8 | A | NC_000008.6 | 119123282 | + | GTGTTCCCATGACANGCTGTTC |
| | | | | | | | | | | ATACTGT |
| 851988 | 10815285 | 24422280 | T | G | 9 | A | NC_000009.6 | 5804424 | + | TTAACTTCACAGATNATAAAAT |
| | | | | | | | | | | TGCTTTC |
| 2413626 | 17571216 | 24561474 | T | C | 9 | A | NC_000009.6 | 5820317 | + | CTAAAACCATGCAGNAACCCTT |
| | | | | | | | | | | GAAGGCA |
| 2413632 | 4628310 | 24098016 | G | A | 9 | A | NC_000009.6 | 5830402 | + | ATGCCATAGCCAAGNGTTCACA |
| | | | | | | | | | | TGTCCAA |
| 2441088 | 3761672 | 24552851 | G | A | 9 | A | NC_000009.6 | 37898658 | + | CATTTCGGTACCCCNGCTTTTT |
| | | | | | | | | | | CTCCCAG |
| 2440329 | 17516973 | 24553177 | C | T | 9 | A | NC_000009.6 | 37958915 | + | GACTCTATGGTATANGGGCCCC |
| | | | | | | | | | | TCTCTGT |
| 2475361 | 12005009 | 23587437 | T | C | 9 | A | NC_000009.6 | 129031799 | + | GGCTGTTTCTCCGGNATCCACG |
| | | | | | | | | | | TGCCTTT |
| 879618 | 17400224 | 24447945 | G | A | 9 | A | NC_000009.6 | 130753983 | + | GCTTCAAAACCCACNCTCCTAG |
| | | | | | | | | | | CACTGTA |
| 2197248 | 7909387 | 23530564 | G | A | 10 | A | NC_000010.5 | 2156622 | + | TGTAAGTTTTGGACNGAAGCTA |
| | | | | | | | | | | CATATGT |
| 2254541 | | | C | G | 10 | A | NC_000010.5 | 58661526 | + | TTGGCTACTGCTATNTCTGAAT |
| | | | | | | | | | | GGCTGTT |
| 1708457 | 7107174 | 24130248 | C | T | 11 | A | NC_000011.5 | 77724244 | + | ATGCAAGGAGAATANGGGACAT |
| | | | | | | | | | | GAGTCCT |
| 1708460 | 4291702 | 24130250 | C | T | 11 | A | NC_000011.5 | 77727556 | + | TAGGGTAGTAATAANGGAGACA |
| | | | | | | | | | | AAAGACA |
| 1745110 | 2282658 | 23573679 | C | G | 11 | A | NC_000011.5 | 104415434 | + | GTTTAGAAATGAAANTGTAGGT |
| | | | | | | | | | | AGATCAC |
| 1747104 | 17106108 | 23611495 | A | G | 11 | A | NC_000011.8 | 106188312 | + | TTCTCCCTTGCTTCNTGGAGTT |
| | | | | | | | | | | ACACTTT |
| 894694 | | | C | G | 12 | A | NC_000012.6 | 5073313 | + | CAGGGCCATCTCTGNTCTCCAG |
| | | | | | | | | | | CTCGTGT |
| 2017765 | 12371357 | 24441595 | G | T | 12 | A | NC_000012.6 | 23054720 | + | CAGAGTAAGTGGTTNTTGTTAA |
| | | | | | | | | | | ATTGAAC |
| 2083538 | | | T | C | 12 | A | NC_000012.6 | 102796808 | + | AACAATAAGACAGANCACAGGA |
| | | | | | | | | | | CCATTTA |
| 2089494 | 7301981 | 24385829 | T | C | 12 | A | NC_000012.6 | 112850871 | + | ATACAGACAGTCTCNGCCTTTT |
| | | | | | | | | | | CTCCTGA |
| 2100009 | 7333215 | 23988205 | T | C | 13 | A | NC_000013.6 | 21077834 | + | TCACATTCTTCAGGNTGAGGCT |
| | | | | | | | | | | CCTGTTC |
| 928560 | 9526717 | 24375758 | A | G | 13 | A | NC_000013.9 | 50530633 | + | AGCAGATAATTGAANCTTCAAG |
| | | | | | | | | | | ATGAAAG |
| 924906 | 9544362 | 24068915 | A | C | 13 | A | NC_000013.6 | 75116603 | + | TTCTCCAGGTAACCNGGCCTGG |
| | | | | | | | | | | GAGCTGA |
| 946520 | 10851158 | 24455916 | T | C | 13 | A | NC_000013.6 | 86384970 | + | ATAAACAGAAAGACNGAAAATA |
| | | | | | | | | | | ATCAGTA |
| 946518 | | | A | G | 13 | A | NC_000013.6 | 86391341 | + | CTCATTGAAAATCTNAGAAGCG |
| | | | | | | | | | | AGCTGAA |
| 946373 | 4772631 | 24456874 | A | G | 13 | A | NC_000013.6 | 86462865 | + | ACTCAAATTAATGCNAAGAAAG |
| | | | | | | | | | | ACATTTA |
| 184250 | 2881770 | 23666679 | A | G | 14 | A | NC_000014.4 | 56633918 | + | GAATAGAAGACCACNAAGTCAC |
| | | | | | | | | | | GCTTTCC |
| 2852925 | 1865997 | 24065977 | T | C | 15 | A | NC_000015.5 | 78207272 | + | ATGCTACTATTTCCNGTTGCCA |
| | | | | | | | | | | TCAAACA |
| 2480214 | 223128 | 24706196 | G | T | 17 | A | NC_000017.6 | 30054978 | + | TTTTGATTCTTGTANCTAATAG |
| | | | | | | | | | | GCAGAGT |
| 821239 | 17717014 | 24595848 | T | A | 17 | A | NC_000017.6 | 49155418 | + | CCTCCTCTTCAACANGACATAT |
| | | | | | | | | | | TCCTCAG |
| 2485721 | 17545743 | 24569351 | A | T | 17 | A | NC_000017.6 | 60922662 | + | ATAATGCTTCAGTGNTGTTGTC |
| | | | | | | | | | | AGAGAGT |
| 1611545 | | | G | A | 19 | A | NC_000019.6 | 45611364 | + | TCAAGGATTTGCACNTTGGCTT |
| | | | | | | | | | | CCTCGAC |
| 435125 | 5011374 | 24547994 | C | A | 20 | A | NC_000020.6 | 9207186 | + | GGTGTTATATAAAANATGATGT |
| | | | | | | | | | | ATATAGC |
| 435134 | 6039424 | 24548027 | C | G | 20 | A | NC_000020.6 | 9220164 | + | ACTCCTGCAGGGAANAAAAAAA |
| | | | | | | | | | | AGGTAAT |
| 421388 | 1984279 | 24690480 | A | C | 20 | A | NC_000020.6 | 23308192 | + | CAGGAAGAAAGAACNATTTCAC |
| | | | | | | | | | | TCACCAA |
| 3352444 | 960190 | 23824090 | T | C | X | X | NC_000023.5 | 18570733 | + | CTTTTCTTCCTCTGNGTGAGTG |
| | | | | | | | | | | GTAGAGC |
| 3883710 | 7878232 | 24727421 | T | G | X | X | NC_000023.5 | 149463988 | + | ACATAATAGAACATNGCATGCA |
| | | | | | | | | | | CAGACTT |

TABLE 1

| | | | | | | GENE |
|---|---|---|---|---|---|---|
| SNP_ID | ACCESSION | POSITION | GENE_ID | GENE_NAME | SNP_GENE_LOC | DESCRIPTION |
| 829480 | NC_000005.5 | 9385019 | 9037 | SEMA5A | intron | sema domain, seven |
| | | | | | | thrombospondin |
| | | | | | | repeats (type 1 and |
| | | | | | | type 1-like), |
| | | | | | | transmembrane |
| | | | | | | domain (TM) and short |
| | | | | | | cytoplasmic domain, |
| | | | | | | (semaphorin) 5A |
| 1032590 | NC_000001.5 | 54007335 | 200008 | LOC200008 | up | hypothetical protein |
| | | | (previously | (previously | | LOC200008 |
| | | | 401952) | LOC401952) | | |
| 1032590 | NC_000001.5 | 54007335 | 51253 | MRPL37 | up (28.4 kb) | mitochondrial |
| | | | | | | ribosomal protein L37 |
| 1032590 | NC_000001.5 | 54007335 | | | | in PARK10 locus |
| 1032596 | NC_000001.5 | 54015180 | 200008 | LOC200008 | intron | hypothetical protein |
| | | | (previously | (previously | | LOC200008 |
| | | | 401952) | LOC401952) | | |
| 1032596 | NC_000001.5 | 54015180 | 51253 | MRPL37 | up (20.6 kb) | mitochondrial |
| | | | | | | ribosomal protein L37 |
| 3025726 | NC_000004.6 | 139365687 | | | | |
| 719978 | NC_000001.5 | 218379723 | | | | |
| 1590687 | NC_000007.8 | 142679898 | 392133 | LOC392133 | nonsynonymous | similar to seven |
| | | | | | coding | transmembrane helix |
| | | | | | change | receptor |
| 1146860 | NC_000001.5 | 176304501 | 64222 | TOR3A | outsideCoding | torsin family 3, |
| | | | | | Region- | member A |
| | | | | | 3′UTR | |
| 3526320 | NC_000004.6 | 125540194 | | | | |
| 1225958 | NC_000002.6 | 11699188 | 9687 | GREB1 | intron | GREB1 protein |
| 1225958 | NC_000002.6 | 11699188 | 9687 | GREB1 | up | GREB1 protein |
| 3883710 | NC_000023.5 | 149463988 | 139135 | LOC139135 | down | hypothetical protein |
| | | | | | | LOC139135 |
| 3883710 | NC_000023.5 | 149463988 | 139135 | PASD1 | down | PASD1 protein |
| 2480214 | NC_000017.6 | 30054978 | 339281 | LOC339281 | down | hypothetical |
| | | | | | | LOC339281 |
| 3113927 | NC_000005.5 | 5407353 | | | | |
| 2812059 | NC_000004.6 | 139331351 | | | | |
| 2382631 | NC_000003.6 | 77670329 | | | | |
| 754013 | NC_000002.6 | 32459104 | 58484 | CARD12 | intron | caspase recruitment |
| | | | | | | domain family, |
| | | | | | | member 12 |
| 3314438 | NC_000005.5 | 111564816 | 114915 | TIGA1 | up | TIGA1 |
| 4040922 | NC_000004.6 | 154283058 | 201798 | TIGD4 | up | tigger transposable |
| | | | | | | element derived 4 |
| 4040922 | NC_000004.6 | 154283058 | 27236 | ARFIP1 | intron | ADP-ribosylation factor |
| | | | | | | interacting protein 1 |
| | | | | | | (arfaptin 1) |
| 2254541 | NC_000010.5 | 58661526 | | | | |
| 1953706 | NC_000008.6 | 93934019 | 286144 | LOC286144 | outsideCoding | hypothetical protein |
| | | | | | Region- | LOC286144 |
| | | | | | 5′UTR | |
| 2197248 | NC_000010.5 | 2156622 | 399707 | LOC399707 | up | hypothetical gene |
| | | | | | | supported by |
| | | | | | | AK056101 |
| 2017765 | NC_000012.6 | 23054720 | | | | |
| 924906 | NC_000013.6 | 75116603 | | | | |
| 567970 | NC_000007.8 | 42105704 | | | | |
| 535278 | NC_000007.8 | 23698145 | | | | |
| 558560 | NC_000007.8 | 36593076 | | | | |
| 2503679 | NC_000003.6 | 190303721 | 285386 | FLJ41238 | intron | FLJ41238 protein |
| 2485721 | NC_000017.6 | 60922662 | 146779 | FLJ25818 | up | hypothetical protein |
| | | | | | | FLJ25818 |
| 1953958 | NC_000008.6 | 94145054 | 389676 | LOC389676 | up | LOC389676 |
| 3413959 | NC_000004.6 | 103372790 | 55024 | BANK1 | intron | B-cell scaffold protein |
| | | | | | | with ankyrin repeats 1 |
| 2441088 | NC_000009.6 | 37898658 | 401506 | LOC401506 | down | LOC401506 |
| 2441088 | NC_000009.6 | 37898658 | 92014 | MCART1 | up | mitochondrial carrier |
| | | | | | | triple repeat 1 |
| 2089494 | NC_000012.6 | 112850871 | | | | |
| 2475361 | NC_000009.6 | 129031799 | 25 | ABL1 | intron | v-abl Abelson murine |
| | | | | | | leukemia viral |
| | | | | | | oncogene homolog 1 |
| 1431787 | NC_000002.6 | 229019671 | | | | |
| 894694 | NC_000012.6 | 5073313 | | | | |
| 651340 | NC_000001.5 | 13502523 | 7799 | PRDM2 | intron | PR domain containing |
| | | | | | | 2, with ZNF domain |
| 4355868 | NC_000006.6 | 162327821 | 5071 | PARK2 | intron | Parkinson disease |
| | | | | | | (autosomal recessive, |
| | | | | | | juvenile) 2, parkin |
| 821239 | NC_000017.6 | 49155418 | 8913 | CACNA1G | intron | calcium channel, |
| | | | | | | voltage-dependent, |
| | | | | | | alpha 1G subunit |
| 1577305 | NC_000007.8 | 84116696 | | | | |
| 1555513 | NC_000007.8 | 22508917 | 3569 | IL6 | up | interleukin 6 |
| | | | | | | (interferon, beta 2) |
| 1128431 | NC_000001.5 | 162787644 | 4259 | MGST3 | up | microsomal glutathione |
| | | | | | | S-transferase 3 |
| 2347338 | NC_000003.6 | 28058788 | | | | |
| 879618 | NC_000009.6 | 130753983 | 64794 | DDX31 | intron | DEAD (Asp-Glu-Ala- |
| | | | | | | Asp) box polypeptide |
| | | | | | | 31 |
| 532112 | NC_000007.8 | 142016288 | 2051 | EPHB6 | up | EphB6 |
| 1427591 | NC_000002.6 | 225609523 | 8452 | CUL3 | intron | cullin 3 |
| 3336262 | NC_000005.5 | 158729574 | 3593 | IL12B | intron | interleukin 12B (natural |
| | | | | | | killer cell stimulatory |
| | | | | | | factor 2, cytotoxic |
| | | | | | | lymphocyte maturation |
| | | | | | | factor 2, p40) |
| 3336262 | NC_000005.5 | 158729574 | 285626 | LOC285626 | up | hypothetical protein |
| | | | | | | LOC285626 |
| 1549117 | NC_000007.8 | 6259487 | 11014 | KDELR2 | intron | KDEL (Lys-Asp-Glu- |
| | | | | | | Leu) endoplasmic |
| | | | | | | reticulum protein |
| | | | | | | retention receptor 2 |
| 1549117 | NC_000007.8 | 6259487 | 54889 | FLJ20306 | up | hypothetical protein |
| | | | | | | FLJ20306 |
| 1611545 | NC_000019.6 | 45611364 | 29950 | SERTAD1 | down | SERTA domain |
| | | | | | | containing 1 |
| 1611545 | NC_000019.6 | 45611364 | 57716 | PRX | up | periaxin |
| 1745110 | NC_000011.5 | 104415434 | 838 | CASP5 | intron | caspase 5, apoptosis- |
| | | | | | | related cysteine |
| | | | | | | protease |
| 1427562 | NC_000002.6 | 225588397 | 8452 | CUL3 | intron | cullin 3 |
| 2651858 | NC_000004.6 | 117844485 | | | | |
| 1270180 | NC_000002.6 | 67598836 | 54465 | ETAA16 | intron | ETAA16 protein |
| 2852925 | NC_000015.5 | 78207272 | 400411 | LOC400411 | down | LOC400411 |
| 1708460 | NC_000011.5 | 77727556 | 9846 | GAB2 | intron | GRB2-associated |
| | | | | | | binding protein 2 |
| 2083538 | NC_000012.6 | 102796808 | | | | |
| 1500751 | NC_000006.6 | 71102255 | | | | |
| 4612764 | NC_000002.6 | 237454681 | | | | |
| 719756 | NC_000001.5 | 218529200 | | | | |
| 726185 | NC_000002.6 | 235130245 | 79054 | TRPM8 | intron | transient receptor |
| | | | | | | potential cation |
| | | | | | | channel, subfamily M, |
| | | | | | | member 8 |
| 3394795 | NC_000004.6 | 5271059 | 55351 | HSA250839 | intron | gene for |
| | | | | | | serine/threonine |
| | | | | | | protein kinase |
| 1708457 | NC_000011.5 | 77724244 | 9846 | GAB2 | intron | GRB2-associated |
| | | | | | | binding protein 2 |
| 2440329 | NC_000009.6 | 37958915 | 6461 | SHB | intron | SHB (Src homology 2 |
| | | | | | | domain containing) |
| | | | | | | adaptor protein B |
| 946520 | NC_000013.6 | 86384970 | | | | |
| 184250 | NC_000014.4 | 56633918 | | | | |
| 3413996 | NC_000004.6 | 103308314 | 55024 | BANK1 | intron | B-cell scaffold protein |
| | | | | | | with ankyrin repeats 1 |
| 362893 | NC_000006.6 | 133540882 | 2070 | EYA4 | up | eyes absent homolog |
| | | | | | | 4 (Drosophila) |
| 3414002 | NC_000004.6 | 103281820 | 55024 | BANK1 | intron | B-cell scaffold protein |
| | | | | | | with ankyrin repeats 1 |
| 1367383 | NC_000002.6 | 166833251 | 2591 | GALNT3 | up | UDP-N-acetyl-alpha-D- |
| | | | | | | galactosamine:polypeptide |
| | | | | | | N- |
| | | | | | | acetylgalactosaminyltransferase |
| | | | | | | 3 (GalNAc- |
| | | | | | | T3) |
| 4417601 | NC_000004.6 | 164852476 | 4889 | NPY5R | down | neuropeptide Y |
| | | | | | | receptor Y5 |
| 946518 | NC_000013.6 | 86391341 | | | | |
| 1276993 | NC_000002.6 | 80767709 | 1496 | CTNNA2 | intron | catenin (cadherin- |
| | | | | | | associated protein), |
| | | | | | | alpha 2 |
| 1481380 | NC_000006.6 | 23958782 | 401238 | LOC401238 | up | similar to chromosome |
| | | | | | | 15 open reading frame 2 |
| 719775 | NC_000001.5 | 218521453 | | | | |
| 3255097 | NC_000003.6 | 30100697 | | | | |
| 435125 | NC_000020.6 | 9207186 | 5332 | PLCB4 | intron | phospholipase C, beta 4 |
| 946373 | NC_000013.6 | 86462865 | | | | |
| 2100009 | NC_000013.6 | 21077834 | 387908 | LOC387908 | up | similar to Ferritin heavy |
| | | | | | | chain (Ferritin H |
| | | | | | | subunit) |
| 840738 | NC_000005.5 | 106917999 | 1946 | EFNA5 | intron | ephrin-A5 |
| 1522974 | NC_000006.6 | 113935407 | | | | |
| 2329698 | NC_000003.6 | 7487728 | 2917 | GRM7 | intron | glutamate receptor, |
| | | | | | | metabotropic 7 |
| 2413632 | NC_000009.6 | 5830402 | 79956 | KIAA1815 | up | KIAA1815 |
| 1980116 | NC_000008.6 | 119123282 | | | | |
| 1903046 | NC_000008.6 | 38459411 | | | | |
| 1481740 | NC_000006.6 | 23795723 | | | | |
| 435134 | NC_000020.6 | 9220164 | 5332 | PLCB4 | intron | phospholipase C, beta 4 |
| 1481718 | NC_000006.6 | 23802517 | | | | |
| 3352444 | NC_000023.5 | 18570733 | | | | |
| 4496903 | NC_000004.6 | 191137918 | | | | |
| 421388 | NC_000020.6 | 23308192 | | | | |
| 2413626 | NC_000009.6 | 5820317 | 79956 | KIAA1815 | intron | KIAA1815 |
| 3393421 | NC_000004.6 | 344051 | 7700 | ZNF141 | intron | zinc finger protein 141 |
| | | | | | | (clone pHZ-44) |
| 851988 | NC_000009.6 | 5804424 | 79956 | KIAA1815 | intron | KIAA1815 |
| 3492068 | NC_000004.6 | 72058145 | 22902 | RIPX | intron | rap2 interacting protein x |
| 1747104 | NC_000011.8 | 106188312 | 2977 | GUCY1A2 | intron | guanylate cyclase 1, |
| | | | | | | soluble, alpha 2 |
| 928560 | NC_000013.9 | 50530633 | 2974 | GUCY1B2 | intron | guanylate cyclase 1, |
| | | | | | | soluble, beta 2 |

| | | | | | FISHER |
|---|---|---|---|---|---|
| | | | | | COMBINED |
| SNP_ID | DELTA_P_ROUND1 | P_STDT_all | DELTA_P_ALL_REP | P_ALL_REP | P_VALUE |
| 829480 | 0.041039454 | 0.001099416 | 0.085584002 | 2.89972E−05 | 6.75473E−07 |
| 1032590 | 0.032418978 | 0.035872795 | 0.091460396 | 0.000351326 | 8.81065E−05 |
| 1032590 | 0.032418978 | 0.035872795 | 0.091460396 | 0.000351326 | 8.81065E−05 |
| 1032590 | 0.032418978 | 0.035872795 | 0.091460396 | 0.000351326 | 8.81065E−05 |
| 1032596 | 0.029910714 | 0.05792767 | 0.085629601 | 0.000876429 | 0.000336722 |
| 1032596 | 0.029910714 | 0.05792767 | 0.085629601 | 0.000876429 | 0.000336722 |
| 3025726 | 0.029265873 | 0.006257629 | 0.051883938 | 0.001084475 | 8.55775E−05 |
| 719978 | −0.044059685 | 0.00601044 | −0.087075209 | 0.00134241 | 0.000295696 |
| 1590687 | −0.037443067 | 0.011616891 | −0.075957709 | 0.002129327 | 0.000522693 |
| 1146860 | −0.033371227 | 0.038597827 | −0.075993535 | 0.004224679 | 0.000772616 |
| 3526320 | −0.034832239 | 0.016581306 | −0.06610286 | 0.004803254 | 0.00038063 |
| 1225958 | 0.034303395 | 0.005564237 | 0.058427985 | 0.004847332 | 0.000348614 |
| 1225958 | 0.034303395 | 0.005564237 | 0.058427985 | 0.004847332 | 0.000348614 |
| 3883710 | 0.050918019 | 0.000959969 | 0.071888791 | 0.005994521 | 0.000227351 |
| 3883710 | 0.050918019 | 0.000959969 | 0.071888791 | 0.005994521 | 0.000227351 |
| 2480214 | 0.043727634 | 0.006195267 | 0.077553159 | 0.006127854 | 0.000570171 |
| 3113927 | −0.025421858 | 0.017676725 | −0.049006623 | 0.00616317 | 0.000653832 |
| 2812059 | 0.027006918 | 0.006695314 | 0.051162791 | 0.006382006 | 0.000355046 |
| 2382631 | 0.047295817 | 0.002860811 | 0.062134705 | 0.006899029 | 0.000156537 |
| 754013 | −0.042111057 | 0.01241133 | −0.074323452 | 0.009867289 | 0.000726949 |
| 3314438 | 0.026497696 | 0.006117153 | 0.050271439 | 0.010360481 | 0.002281944 |
| 4040922 | −0.038035883 | 0.032007669 | −0.070514636 | 0.012657325 | 0.003061909 |
| 4040922 | −0.038035883 | 0.032007669 | −0.070514636 | 0.012657325 | 0.003061909 |
| 2254541 | 0.02787057 | 0.003433193 | 0.038965039 | 0.013975469 | 0.000509179 |
| 1953706 | −0.009028972 | 0.032509445 | −0.018330583 | 0.014846852 | 0.003686551 |
| 2197248 | −0.020358962 | 0.024448945 | −0.034879204 | 0.015318395 | 0.002441241 |
| 2017765 | 0.059454792 | 0.000959969 | 0.066760037 | 0.016530267 | 0.000362946 |
| 924906 | −0.022207659 | 0.059346439 | −0.065770308 | 0.01660802 | 0.00577891 |
| 567970 | 0.029388201 | 0.002250227 | 0.04306017 | 0.016983769 | 0.000288243 |
| 535278 | −0.01763594 | 0.009023439 | −0.026155116 | 0.017357141 | 0.002013366 |
| 558560 | −0.02593869 | 0.027148285 | −0.046335798 | 0.018114806 | 0.001445532 |
| 2503679 | 0.0456621 | 0.004764244 | 0.065822251 | 0.018537272 | 0.000726132 |
| 2485721 | 0.033003283 | 0.01067052 | 0.053879271 | 0.018915579 | 0.001414806 |
| 1953958 | 0.021852866 | 0.089555074 | 0.058505107 | 0.019289066 | 0.007573753 |
| 3413959 | 0.03175071 | 0.001353864 | 0.04086351 | 0.020471954 | 0.000356806 |
| 2441088 | −0.021709555 | 0.073124486 | −0.063864152 | 0.020719755 | 0.007259468 |
| 2441088 | −0.021709555 | 0.073124486 | −0.063864152 | 0.020719755 | 0.007259468 |
| 2089494 | 0.046973164 | 0.002436735 | 0.064742963 | 0.021731169 | 0.000527032 |
| 2475361 | −0.03545876 | 0.009036479 | −0.051518198 | 0.024405605 | 0.001263616 |
| 1431787 | 0.026673507 | 0.008774633 | 0.040054423 | 0.0245165 | 0.002063737 |
| 894694 | 0.037473706 | 0.008404418 | 0.063971706 | 0.024939265 | 0.000698178 |
| 651340 | 0.042267111 | 0.000636299 | 0.048073198 | 0.025107596 | 0.000534812 |
| 4355868 | 0.026878744 | 0.033894854 | 0.065911748 | 0.027737204 | 0.004917207 |
| 821239 | −0.036938145 | 0.013642709 | −0.059487354 | 0.027777623 | 0.001009303 |
| 1577305 | 0.026057906 | 0.066057019 | 0.055539671 | 0.027973256 | 0.006933803 |
| 1555513 | 0.029649616 | 0.04833224 | 0.062669437 | 0.028048028 | 0.007024178 |
| 1128431 | −0.035844949 | 0.028503434 | −0.061246549 | 0.029494878 | 0.01035083 |
| 2347338 | 0.039752207 | 0.003184204 | 0.053877974 | 0.029509355 | 0.000478756 |
| 879618 | 0.05530303 | 0.001966751 | 0.058758387 | 0.030010063 | 0.000755362 |
| 532112 | 0.030853821 | 0.025696419 | 0.059375406 | 0.030444903 | 0.004483589 |
| 1427591 | 0.033234054 | 0.001876415 | 0.042617462 | 0.030555039 | 0.000660274 |
| 3336262 | 0.030712003 | 0.028943138 | 0.048383218 | 0.03175405 | 0.007295716 |
| 3336262 | 0.030712003 | 0.028943138 | 0.048383218 | 0.03175405 | 0.007295716 |
| 1549117 | −0.030174662 | 0.023954799 | −0.050381072 | 0.032054812 | 0.004874476 |
| 1549117 | −0.030174662 | 0.023954799 | −0.050381072 | 0.032054812 | 0.004874476 |
| 1611545 | −0.006076607 | 0.058781721 | −0.012823618 | 0.033692606 | 0.00771276 |
| 1611545 | −0.006076607 | 0.058781721 | −0.012823618 | 0.033692606 | 0.00771276 |
| 1745110 | 0.046713045 | 0.00280161 | 0.057158589 | 0.034559656 | 0.001672032 |
| 1427562 | 0.030186106 | 0.008049303 | 0.04135525 | 0.035834026 | 0.002961647 |
| 2651858 | −0.029185895 | 0.007218325 | −0.039778297 | 0.036895339 | 0.002464223 |
| 1270180 | −0.023850644 | 0.044005832 | −0.054880186 | 0.03748915 | 0.012360749 |
| 2852925 | 0.034983396 | 0.017692195 | 0.055984681 | 0.037590382 | 0.003092457 |
| 1708460 | 0.030206661 | 0.007805044 | 0.043057255 | 0.040640883 | 0.007781603 |
| 2083538 | −0.032453058 | 0.002699796 | −0.040029433 | 0.041972179 | 0.001713968 |
| 1500751 | −0.039846766 | 0.002022049 | −0.044388517 | 0.044341915 | 0.000742388 |
| 4612764 | −0.009773169 | 0.052203635 | −0.021562113 | 0.044854898 | 0.015745844 |
| 719756 | −0.055734229 | 0.000711811 | −0.055030717 | 0.045319951 | 0.000340876 |
| 726185 | 0.020752743 | 0.042840247 | 0.04599835 | 0.046001658 | 0.009646668 |
| 3394795 | 0.008057715 | 0.020921335 | 0.013138962 | 0.0460428 | 0.001864412 |
| 1708457 | 0.02828758 | 0.013765577 | 0.041566691 | 0.046138912 | 0.011802343 |
| 2440329 | −0.023944121 | 0.022740296 | −0.033360927 | 0.048150558 | 0.003490868 |
| 946520 | 0.052887831 | 0.016462313 | 0.06172604 | 0.048244445 | 0.003112405 |
| 184250 | −0.035491071 | 0.034069195 | −0.055512271 | 0.048285616 | 0.013844476 |
| 3413996 | 0.032516704 | 0.000480875 | 0.034579181 | 0.049247721 | 0.000314144 |
| 362893 | 0.039402174 | 0.013953435 | 0.057169149 | 0.049533829 | 0.007106629 |
| 3414002 | 0.036873249 | 4.25559E−05 | 0.034376127 | 0.050228773 | 3.85357E−05 |
| 1367383 | −0.022533023 | 0.005905392 | −0.033094187 | 0.05475783 | 0.000961754 |
| 4417601 | 0.019954133 | 0.000238563 | 0.015610876 | 0.055461988 | 0.000168886 |
| 946518 | 0.061044882 | 0.004139419 | 0.05467323 | 0.065682208 | 0.000915593 |
| 1276993 | 0.043622848 | 0.002869113 | 0.053685688 | 0.073299276 | 0.000828373 |
| 1481380 | 0.053284753 | 0.000364932 | 0.047414992 | 0.079450545 | 0.000197749 |
| 719775 | −0.056661167 | 0.001088217 | −0.047962705 | 0.085144179 | 0.000938456 |
| 3255097 | −0.046045117 | 0.000182811 | −0.040504886 | 0.091042221 | 0.000170964 |
| 435125 | 0.059659033 | 0.000711811 | 0.046131684 | 0.123016504 | 0.000711076 |
| 946373 | 0.049907004 | 0.003664669 | 0.042912616 | 0.129516014 | 0.000982133 |
| 2100009 | −0.038014564 | 0.000520244 | −0.029028698 | 0.13464635 | 0.000336203 |
| 840738 | −0.059684685 | 0.000229823 | −0.042546755 | 0.137917872 | 0.000363432 |
| 1522974 | −0.034536207 | 0.000336194 | −0.02210026 | 0.176755821 | 0.000519715 |
| 2329698 | 0.056768822 | 0.000212183 | 0.035198352 | 0.180194688 | 0.000322463 |
| 2413632 | 0.052083333 | 0.000167827 | 0.025864606 | 0.233403516 | 0.000291398 |
| 1980116 | 0.048760041 | 8.86109E−05 | 0.023574319 | 0.253768211 | 0.000216366 |
| 1903046 | 0.051502715 | 1.63093E−05 | 0.022938326 | 0.262071957 | 0.000119667 |
| 1481740 | 0.053476522 | 0.000193942 | 0.024027036 | 0.379335669 | 0.000605673 |
| 435134 | 0.059462781 | 9.55176E−05 | 0.023325859 | 0.407812673 | 0.00037126 |
| 1481718 | 0.051959187 | 0.000125654 | 0.021661919 | 0.421582156 | 0.000673717 |
| 3352444 | −0.016697358 | 0.025347319 | −3.45877E−05 | 0.596455025 | 0.000338681 |
| 4496903 | −0.057724791 | 5.83557E−05 | −0.010861423 | 0.649173539 | 0.000423598 |
| 421388 | −0.06308661 | 4.82983E−05 | −0.00836626 | 0.759087757 | 0.000337135 |
| 2413626 | 0.062703636 | 4.46777E−05 | 0.00840209 | 0.761070296 | 0.000791771 |
| 3393421 | 0.052797217 | 0.000568531 | 0.006004538 | 0.809228049 | 0.00077559 |
| 851988 | 0.062670702 | 1.96272E−05 | 0.002461591 | 0.929546039 | 0.000476427 |
| 3492068 | 0.023738463 | 9.63509E−05 | 0.00076977 | 0.939165907 | 0.000822247 |
| 1747104 | 0.025507 | 0.000607 | | | 0.002178 |
| 928560 | 0.05291 | 0.006099 | | | 0.008061 |

TABLE 4
Genomic SNPs associated with PD in two samples (tier 1 and tier 2)

| | | NCBI | | | | | | | |
|---|---|---|---|---|---|---|---|---|---|
| dbSNP | Gene | Build | | Gene ontology^{b} | | | | OR^{d} | |
| rs # | name | 35.1 | Brain^{a} | Function | Process | Component | Summary^{b,c} | (95% CI) | P value^{d} |
| 7702187 | SEMA5A | 5p15.2 | yes | Receptor activity | Cell adhesion; | Integral to | Axonal guidance (neural | 1.74 | 7.62E−06 |
| | | | | | cell-cell | membrane | development); initiation | (1.36-2.24) | |
| | | | | | signaling; | | of neuronal | | |
| | | | | | neurogenesis | | apoptosis | | |
| 10200894 | | 2q36 | | | | | PARK11 locus | 1.84 | 1.70E−05 |
| | | | | | | | | (1.38-2.45) | |
| 2313982 | | 4q31.1 | | | | | | 2.01 | 1.79E−05 |
| | | | | | | | | (1.44-2.79) | |
| 17329669 | | 7p14 | | | | | | 1.71 | 2.30E−05 |
| | | | | | | | | (1.33-2.21) | |
| 7723605 | | 5p15.3 | | | | | | 1.78 | 3.30E−05 |
| | | | | | | | | (1.35-2.35) | |
| 2254541^{e} | | | | | | | | 1.88 | 3.65E−05 |
| | | | | | | | | (1.38-2.57) | |
| 16851009 | GALNT3 | 2q24 | yes | Manganese, sugar | Carbohydrate | Golgi | Marker of differentiation | 1.84 | 4.17E−05 |
| | | | | binding; transferase | metabolism | apparatus; | and aggressiveness | (1.36-2.49) | |
| | | | | activity | | integral to | (several cancers) | | |
| | | | | | | membrane | | | |
| 2245218 | PRDM2 | 1p36.2 | yes | DNA, metal, zinc | Transcription | Nucleus | Tumor suppression; | 1.67 | 4.61E−05 |
| | | | | binding; | regulation | | neuronal differentiation; | (1.29-2.14) | |
| | | | | transcription factor, | | | estrogen receptor | | |
| | | | | regulator activity | | | binding; estrogen effector | | |
| 7878232 | PASD1 | Xq28 | yes | Signal transducer | Signal | | X-linked | 1.38 | 6.87E−05 |
| | | | | activity | transduction | | | (1.17-1.62) | |
| 1509269 | | 4q31.1 | | | | | | 1.71 | 9.21E−05 |
| | | | | | | | | (1.30-2.26) | |
| 11737074 | | 4q27 | | | | | | 1.50 | 1.55E−04 |
| | | | | | | | | (1.21-1.86) | |

^{a}Annotated from GeneCards (http://bioinfo.weizmann.ac.il/cards/index.shtml). Evidence for brain expression.
^{b}Annotated from Entrez Gene (http://www.ncbi.nlm.nih.gov/entrez/query.fcgi?db=gene). Summary of biological plausibility.
^{c}Annotated from OMIM (http://www.ncbi.nlm.nih.gov/entrez/query.fcgi?CMD=search&DB=omim).
^{d}Derived for the combined tier 1 and tier 2 samples, using a liberalization of the sibling transmission disequilibrium test (log additive model).
^{e}This SNP is designated with a Perlegen Sciences internal SNP identifier; it has been submitted to dbSNP and rs# and cytogenetic location are not yet available.

TABLE 5
Criteria for SNPs selected for genotyping in tier 2

| Category | Description | SNPs |
|---|---|---|
| 1 | P < 0.01 in tier 1 overall analysis | 1,525 |
| 2 | P < 0.001 in tier 1 age or gender stratified analyses | 188 |
| 3 | P < 0.05 in tier 1 overall analysis, within +/−10 kb | 8 |
| | of the six linkage-derived candidate genes | |
| | (SNCA, PARK2, UCHL1, MAPT, DJ1, PINK1) | |
| 4 | P < 0.05 in tier 1 overall analysis, within | 159 |
| | additional PARK linkage loci for which genes | |
| | have not been cloned (PARK3, PARK8, PARK9, | |
| | PARK10, PARK11) | |
| 5 | P < 0.05 in tier 1 overall analysis, within | 619 |
| | exons or within 10 kb 5′ of the transcript, | |
| | genome wide | |
| 6 | Genomic control SNPs | 311 |
| 7 | Other SNPs in LD with those passing | 338 |
| | criteria 1 and 2 | |
| | Total | 3,148 |

1. An isolated nucleic acid that specifically hybridizes to a genomic sequence from 10 kb upstream to 10 kb downstream of a PD-related disease nucleic acid, for use in diagnostics, prognostics, prevention, treatment, or study of PD-related disease, wherein said PD-related disease nucleic acid contains a base at a position selected from the group of base positions shown in Table 1.

2. A nucleic acid of claim 1 wherein the PD-related disease is Parkinson's disease.

3-10. (canceled)

11. A method of detecting presence of or susceptibility to PD-related disease in a patient, comprising determining whether the patient contains a polymorphic form of a protein encoded by a nucleic acid of claim 1 and polymorphic forms in linkage disequilibrium with any of these, the presence of the polymorphic form indicating presence or susceptibility to PD-related disease.

12. The method of claim 11 wherein the PD-related disease is Parkinson's disease.

13. A method of inhibiting or treating PD-related disease, comprising administering to a patient suffering from or at risk of PD-related disease an agent that modulates expression or activity of a protein encoded by a nucleic acid of claim 1 in a regime effective to inhibit or treat the PD-related disease in the patient.

14. The method of claim 13 further comprising monitoring a property of the brain in the patient responsive to the administration.

15. The method of claim 13 wherein the patient is a human.

16. The method of claim 13 wherein the PD-related disease is Parkinson's disease.

17-18. (canceled)


SEQUENCE LISTING (94 sequences)

SEQ ID NO: 1 - DNA, 29 bp, Homo sapiens
ccggagccttggtcytcggtatttaacat

SEQ ID NO: 2 - DNA, 29 bp, Homo sapiens
acaacacgcagtggrcatcatcagaggaa

SEQ ID NO: 3 - DNA, 29 bp, Homo sapiens
agggaacataacagyatagcccatcctgt

SEQ ID NO: 4 - DNA, 29 bp, Homo sapiens
gagtagatatatgtwtttgagaatgagag

SEQ ID NO: 5 - DNA, 29 bp, Homo sapiens
aaatttttgattttyctaaaatccttggc

SEQ ID NO: 6 - DNA, 29 bp, Homo sapiens
taggtagtggggagwcaaaggtagcttag

SEQ ID NO: 7 - DNA, 29 bp, Homo sapiens
gaaagtttggaaaayttaatcaaaactta

SEQ ID NO: 8 - DNA, 29 bp, Homo sapiens
tgccatatctaaacytcaaaatttcttaa

SEQ ID NO: 9 - DNA, 29 bp, Homo sapiens
aggtggcaagcttcrtattggtaagaatt

SEQ ID NO: 10 - DNA, 29 bp, Homo sapiens
cttattttcttgcayattcaatgtaagtt

SEQ ID NO: 11 - DNA, 29 bp, Homo sapiens
acgcggacctgactrctacccgaacaacc

SEQ ID NO: 12 - DNA, 29 bp, Homo sapiens
ttttcatgtgtcctsctagctagtttttt

SEQ ID NO: 13 - DNA, 29 bp, Homo sapiens
catcaacctgtgttyacctcctttctgtt

SEQ ID NO: 14 - DNA, 29 bp, Homo sapiens
aatatttttaatagycattatgttacaga

SEQ ID NO: 15 - DNA, 29 bp, Homo sapiens
gatatagcttttctygcatttttaaaaat

SEQ ID NO: 16 - DNA, 29 bp, Homo sapiens
tcttatacactttcsaatatgctgtacta

SEQ ID NO: 17 - DNA, 29 bp, Homo sapiens
actcatttctgctcytattgtgtttttag

SEQ ID NO: 18 - DNA, 29 bp, Homo sapiens
gagcaaaggagcttrgctcttccatgcca

SEQ ID NO: 19 - DNA, 29 bp, Homo sapiens
aacattcctgaaccrcaattcccttcttt

SEQ ID NO: 20 - DNA, 29 bp, Homo sapiens
gtctcagtgtagctygtcaaccctgaagt

SEQ ID NO: 21 - DNA, 29 bp, Homo sapiens
actttgacagtcacrcagtctgctttcta

SEQ ID NO: 22 - DNA, 29 bp, Homo sapiens
aatgctctactcgaytgaaaaagactttt

SEQ ID NO: 23 - DNA, 29 bp, Homo sapiens
cagcaaatagtgagraaagttgaatctta

SEQ ID NO: 24 - DNA, 29 bp, Homo sapiens
ctatgccatatgttscgatatgttctatg

SEQ ID NO: 25 - DNA, 29 bp, Homo sapiens
gttttaagcaaaatrtagtctcagctctg

SEQ ID NO: 26 - DNA, 29 bp, Homo sapiens
aatccctggaaaagyagaatgtacccgat

SEQ ID NO: 27 - DNA, 29 bp, Homo sapiens
gaaattatcttcctygtgatcattatcta

SEQ ID NO: 28 - DNA, 29 bp, Homo sapiens
cagtattggaccacrtcgctaaataagcc

SEQ ID NO: 29 - DNA, 29 bp, Homo sapiens
tgggaagtaattctraaacaggaggccta

SEQ ID NO: 30 - DNA, 29 bp, Homo sapiens
accaaattttgccaygtactgacttggca

SEQ ID NO: 31 - DNA, 29 bp, Homo sapiens
gtagaagtagtaacrcacaggcataacaa

SEQ ID NO: 32 - DNA, 29 bp, Homo sapiens
aattcaatctcaagyccatctggctcctg

SEQ ID NO: 33 - DNA, 29 bp, Homo sapiens
ccatagtgctattcrtgtgttttcagtcc

SEQ ID NO: 34 - DNA, 29 bp, Homo sapiens
ttccttgatctttastagcttttggcagt

SEQ ID NO: 35 - DNA, 29 bp, Homo sapiens
tttttcttctctcawtctcaccatggaca

SEQ ID NO: 36 - DNA, 29 bp, Homo sapiens
cgccattgttttaarcactatcctacttt

SEQ ID NO: 37 - DNA, 29 bp, Homo sapiens
ggctaaacgtgactygaagtttcagtgta

SEQ ID NO: 38 - DNA, 29 bp, Homo sapiens
gcagtatggactggwgcttacatcatgtc

SEQ ID NO: 39 - DNA, 29 bp, Homo sapiens
ctcttgagcacttaygctttatttgtgta

SEQ ID NO: 40 - DNA, 29 bp, Homo sapiens
tgtatgaatatggaygcaatataataaca

SEQ ID NO: 41 - DNA, 29 bp, Homo sapiens
ctgattatgtctttwtgcacttggagaaa

SEQ ID NO: 42 - DNA, 29 bp, Homo sapiens
gccttagcaaatgayagtaattctgaaaa

SEQ ID NO: 43 - DNA, 29 bp, Homo sapiens
aatggtcagaaaagycagtttagtccatt

SEQ ID NO: 44 - DNA, 29 bp, Homo sapiens
aaaaactaaactttraactgattctgagc

SEQ ID NO: 45 - DNA, 29 bp, Homo sapiens
ttgaagatgagagtrctaaaaatctgtga

SEQ ID NO: 46 - DNA, 29 bp, Homo sapiens
attgaggacatacayagtttatgctagaa

SEQ ID NO: 47 - DNA, 29 bp, Homo sapiens
gttaacaatatgctraagtatgaattttt

SEQ ID NO: 48 - DNA, 29 bp, Homo sapiens
atttcttttccccartcacctaactcagt

SEQ ID NO: 49 - DNA, 29 bp, Homo sapiens
aggtatgaacccacractacagctggtct

SEQ ID NO: 50 - DNA, 29 bp, Homo sapiens
tagttgtgtcttgcsatgctaaaggacgt

SEQ ID NO: 51 - DNA, 29 bp, Homo sapiens
agtcgtccttgtcgwaggagtttcaggaa

SEQ ID NO: 52 - DNA, 29 bp, Homo sapiens
tatatctcccatggrtcaattaggtgttg

SEQ ID NO: 53 - DNA, 29 bp, Homo sapiens
gatgtaagtaactcyagagcataggaaat

SEQ ID NO: 54 - DNA, 29 bp, Homo sapiens
caataactttccagygatgccaataatcc

SEQ ID NO: 55 - DNA, 29 bp, Homo sapiens
aacaggtcatgatgrttcagaaaagcaaa

SEQ ID NO: 56 - DNA, 29 bp, Homo sapiens
gccttgaaggagaargccacagggcacgg

SEQ ID NO: 57 - DNA, 29 bp, Homo sapiens
attatttgatgtcarcttagcattatcaa

SEQ ID NO: 58 - DNA, 29 bp, Homo sapiens
cattggggctcctcyagcaataaattgct

SEQ ID NO: 59 - DNA, 29 bp, Homo sapiens
accaaggagctggtyccatagtggattgt

SEQ ID NO: 60 - DNA, 29 bp, Homo sapiens
gtgttcccatgacaygctgttcatactgt

SEQ ID NO: 61 - DNA, 29 bp, Homo sapiens
ttaacttcacagatkataaaattgctttc

SEQ ID NO: 62 - DNA, 29 bp, Homo sapiens
ctaaaaccatgcagyaacccttgaaggca

SEQ ID NO: 63 - DNA, 29 bp, Homo sapiens
atgccatagccaagrgttcacatgtccaa

SEQ ID NO: 64 - DNA, 29 bp, Homo sapiens
catttcggtaccccrgctttttctcccag

SEQ ID NO: 65 - DNA, 29 bp, Homo sapiens
gactctatggtataygggcccctctctgt

SEQ ID NO: 66 - DNA, 29 bp, Homo sapiens
ggctgtttctccggyatccacgtgccttt

SEQ ID NO: 67 - DNA, 29 bp, Homo sapiens
gcttcaaaacccacrctcctagcactgta

SEQ ID NO: 68 - DNA, 29 bp, Homo sapiens
tgtaagttttggacrgaagctacatatgt

SEQ ID NO: 69 - DNA, 29 bp, Homo sapiens
ttggctactgctatstctgaatggctgtt

SEQ ID NO: 70 - DNA, 29 bp, Homo sapiens
atgcaaggagaataygggacatgagtcct

SEQ ID NO: 71 - DNA, 29 bp, Homo sapiens
tagggtagtaataayggagacaaaagaca

SEQ ID NO: 72 - DNA, 29 bp, Homo sapiens
gtttagaaatgaaastgtaggtagatcac

SEQ ID NO: 73 - DNA, 29 bp, Homo sapiens
ttctcccttgcttcrtggagttacacttt

SEQ ID NO: 74 - DNA, 29 bp, Homo sapiens
cagggccatctctgstctccagctcgtgt

SEQ ID NO: 75 - DNA, 29 bp, Homo sapiens
cagagtaagtggttkttgttaaattgaac

SEQ ID NO: 76 - DNA, 29 bp, Homo sapiens
aacaataagacagaycacaggaccattta

SEQ ID NO: 77 - DNA, 29 bp, Homo sapiens
atacagacagtctcygccttttctcctga

SEQ ID NO: 78 - DNA, 29 bp, Homo sapiens
tcacattcttcaggytgaggctcctgttc

SEQ ID NO: 79 - DNA, 29 bp, Homo sapiens
agcagataattgaarcttcaagatgaaag

SEQ ID NO: 80 - DNA, 29 bp, Homo sapiens
ttctccaggtaaccmggcctgggagctga

SEQ ID NO: 81 - DNA, 29 bp, Homo sapiens
ataaacagaaagacygaaaataatcagta

SEQ ID NO: 82 - DNA, 29 bp, Homo sapiens
ctcattgaaaatctragaagcgagctgaa

SEQ ID NO: 83 - DNA, 29 bp, Homo sapiens
actcaaattaatgcraagaaagacattta

SEQ ID NO: 84 - DNA, 29 bp, Homo sapiens
gaatagaagaccacraagtcacgctttcc

SEQ ID NO: 85 - DNA, 29 bp, Homo sapiens
atgctactatttccygttgccatcaaaca

SEQ ID NO: 86 - DNA, 29 bp, Homo sapiens
ttttgattcttgtakctaataggcagagt

SEQ ID NO: 87 - DNA, 29 bp, Homo sapiens
cctcctcttcaacawgacatattcctcag

SEQ ID NO: 88 - DNA, 29 bp, Homo sapiens
ataatgcttcagtgwtgttgtcagagagt

SEQ ID NO: 89 - DNA, 29 bp, Homo sapiens
tcaaggatttgcacrttggcttcctcgac

SEQ ID NO: 90 - DNA, 29 bp, Homo sapiens
ggtgttatataaaamatgatgtatatagc

SEQ ID NO: 91 - DNA, 29 bp, Homo sapiens
actcctgcagggaasaaaaaaaaggtaat

SEQ ID NO: 92 - DNA, 29 bp, Homo sapiens
caggaagaaagaacmatttcactcaccaa

SEQ ID NO: 93 - DNA, 29 bp, Homo sapiens
cttttcttcctctgygtgagtggtagagc

SEQ ID NO: 94 - DNA, 29 bp, Homo sapiens
acataatagaacatkgcatgcacagactt